SEQUENCE LISTING (127 sequences)

SEQ ID NO: 1 - DNA, 12 bp, Artificial Sequence
  Synthetic
  misc_feature (1)..(12): n is a, c, g, or t
nnnnnnnnnnnn

SEQ ID NO: 2 - DNA, 37 bp, Artificial Sequence
  Synthetic
atatatgctcttctagtatgcaggttcaactggtgga

SEQ ID NO: 3 - DNA, 36 bp, Artificial Sequence
  Synthetic
tatatagctcttcatgcagagctcaccgtcacctga

SEQ ID NO: 4 - DNA, 36 bp, Artificial Sequence
  Synthetic
atatatgctcttctagtatggcacaggttcagctgg

SEQ ID NO: 5 - DNA, 37 bp, Artificial Sequence
  Synthetic
tatatagctcttcatgctgtaaacgggctgctaacgg

SEQ ID NO: 6 - DNA, 72 bp, Artificial Sequence
  Synthetic
agctctgcaggaagagctgctgtctcttatacacatctgacgctgccgacgagctacccgtacgacgttccg

SEQ ID NO: 7 - DNA, 72 bp, Artificial Sequence
  Synthetic
cggaacgtcgtacgggtagctcgtcggcagcgtcagatgtgtataagagacagcagctcttcctgcagagct

SEQ ID NO: 8 - DNA, 69 bp, Artificial Sequence
  Synthetic
agctctgcaggaagagcttcgtcggcagcgtcagatgtgtataagagacagtacccgtacgacgttccg

SEQ ID NO: 9 - DNA, 69 bp, Artificial Sequence
  Synthetic
cggaacgtcgtacgggtactgtctcttatacacatctgacgctgccgacgaagctcttcctgcagagct

SEQ ID NO: 10 - DNA, 72 bp, Artificial Sequence
  Synthetic
gtgtctgcaggaagagctgctgtctcttatacacatctgacgctgccgacgagctacccgtacgacgttccg

SEQ ID NO: 11 - DNA, 72 bp, Artificial Sequence
  Synthetic
cggaacgtcgtacgggtagctcgtcggcagcgtcagatgtgtataagagacagcagctcttcctgcagacac

SEQ ID NO: 12 - DNA, 57 bp, Artificial Sequence
  Synthetic
aacagtctgaagccggaggataccgcggtgtattattgcaatgtcaacgtggggttt

SEQ ID NO: 13 - DNA, 57 bp, Artificial Sequence
  Synthetic
aaaccccacgttgacattgcaataatacaccgcggtatcctccggcttcagactgtt

SEQ ID NO: 14 - DNA, 20 bp, Artificial Sequence
  Synthetic
gacaacgcctgtagcattcc

SEQ ID NO: 15 - DNA, 51 bp, Artificial Sequence
  Synthetic
tcgtcggcagcgtcagatgtgtataagagacagcagcctgcgcctgagctg

SEQ ID NO: 16 - DNA, 56 bp, Artificial Sequence
  Synthetic
gtctcgtgggctcggagatgtgtataagagacagcctgggtgccctggccccaata

SEQ ID NO: 17 - DNA, 22 bp, Artificial Sequence
  Synthetic
aatgatacggcgaccaccgaga

SEQ ID NO: 18 - DNA, 55 bp, Artificial Sequence
  Synthetic
gtctcgtgggctcggagatgtgtataagagacaggataccgcggtgtattattgc

SEQ ID NO: 19 - DNA, 37 bp, Artificial Sequence
  Synthetic
atatatgctcttctagtatgcaggtccagctccaaga

SEQ ID NO: 20 - DNA, 37 bp, Artificial Sequence
  Synthetic
tatatagctcttcatgcgctcgacaccgttacttgtg

SEQ ID NO: 21 - DNA, 40 bp, Artificial Sequence
  Synthetic
atatatgctcttctagtatggaagttcaacttgtagagag

SEQ ID NO: 22 - DNA, 36 bp, Artificial Sequence
  Synthetic
tatatagctcttcatgcgctgctcacggtgacctgg

SEQ ID NO: 23 - DNA, 36 bp, Artificial Sequence
  Synthetic
tatatagctcttcatgcgctgctcactgttacctgg

SEQ ID NO: 24 - DNA, 43 bp, Artificial Sequence
  Synthetic
cgcggtgtattattgcgcaaaggacgcggacctggtatggtac

SEQ ID NO: 25 - DNA, 43 bp, Artificial Sequence
  Synthetic
gtaccataccaggtccgcgtcctttgcgcaataatacaccgcg

SEQ ID NO: 26 - DNA, 43 bp, Artificial Sequence
  Synthetic
cgcggtgtattattgcgctaaagacgcggacctggtatggtac

SEQ ID NO: 27 - DNA, 43 bp, Artificial Sequence
  Synthetic
gtaccataccaggtccgcgtctttagcgcaataatacaccgcg

SEQ ID NO: 28 - DNA, 46 bp, Artificial Sequence
  Synthetic
cggacaaggaacacaagttacggtaagcagcgcaggaagagctgct

SEQ ID NO: 29 - DNA, 46 bp, Artificial Sequence
  Synthetic
agcagctcttcctgcgctgcttaccgtaacttgtgttccttgtccg

SEQ ID NO: 30 - DNA, 49 bp, Artificial Sequence
  Synthetic
aaccggacaaggaacacaggtcactgtaagcagcgcaggaagagctgct

SEQ ID NO: 31 - DNA, 49 bp, Artificial Sequence
  Synthetic
agcagctcttcctgcgctgcttacagtgacctgtgttccttgtccggtt

SEQ ID NO: 32 - DNA, 45 bp, Artificial Sequence
  Synthetic
  misc_feature (23)..(26): N is any nucleotide
cgcggcgagcggcwmtatttytnnnnatgggctggtatcgccagg

SEQ ID NO: 33 - DNA, 74 bp, Artificial Sequence
  Synthetic
  misc_feature (37)..(37): N is any nucleotide
  misc_feature (45)..(45): n is a, c, g, or t
ccgggcaaagaacgcgaayttgttgccrstattrvtnggtrstantaccwattatgcggatagcgtgaaaggcc

SEQ ID NO: 34 - DNA, 52 bp, Artificial Sequence
  Synthetic
  misc_feature (24)..(30): N is any nucleotide
  misc_feature (34)..(34): N is any nucleotide
ccgcggtgtattattgcgcggytnnnnnnnywtntattggggccagggcacc

SEQ ID NO: 35 - DNA, 56 bp, Artificial Sequence
  Synthetic
  misc_feature (24)..(34): N is any nucleotide
  misc_feature (38)..(38): N is any nucleotide
ccgcggtgtattattgcgcggytnnnnnnnnnnnywtntattggggccagggcacc

SEQ ID NO: 36 - DNA, 60 bp, Artificial Sequence
  Synthetic
  misc_feature (24)..(38): N is any nucleotide
  misc_feature (42)..(42): N is any nucleotide
ccgcggtgtattattgcgcggytnnnnnnnnnnnnnnnywtntattggggccagggcacc

SEQ ID NO: 37 - DNA, 50 bp, Artificial Sequence
  Synthetic
caggtgcagctgcaggaaagcggcggcggcctggtgcaggcgggcggcag

SEQ ID NO: 38 - DNA, 43 bp, Artificial Sequence
  Synthetic
gccgctcgccgcgcagctcaggcgcaggctgccgcccgcctgc

SEQ ID NO: 39 - DNA, 39 bp, Artificial Sequence
  Synthetic
ttcgcgttctttgcccggcgcctggcgataccagcccat

SEQ ID NO: 40 - DNA, 54 bp, Artificial Sequence
  Synthetic
gtttttcgcgttatcgcggctaatggtaaagcggcctttcacgctatccgcata

SEQ ID NO: 41 - DNA, 53 bp, Artificial Sequence
  Synthetic
agccgcgataacgcgaaaaacaccgtgtatctgcagatgaacagcctgaaacc

SEQ ID NO: 42 - DNA, 52 bp, Artificial Sequence
  Synthetic
cgcgcaataatacaccgcggtatcttccggtttcaggctgttcatctgcaga

SEQ ID NO: 43 - DNA, 36 bp, Artificial Sequence
  Synthetic
gctgctcacggtcacctgggtgccctggccccaata

SEQ ID NO: 44 - DNA, 37 bp, Artificial Sequence
  Synthetic
atatatgctcttctagtcaggtgcagctgcaggaaag

SEQ ID NO: 45 - DNA, 36 bp, Artificial Sequence
  Synthetic
tatatagctcttcatgcgctgctcacggtcacctgg

SEQ ID NO: 46 - DNA, 51 bp, Artificial Sequence
  Synthetic
aatgatacggcgaccaccgagatctacacctctctattcgtcggcagcgtc

SEQ ID NO: 47 - DNA, 52 bp, Artificial Sequence
  Synthetic
gtctcgtgggctcggagatgtgtataagagacagagctgtgccgcaagcggt

SEQ ID NO: 48 - DNA, 52 bp, Artificial Sequence
  Synthetic
gtctcgtgggctcggagatgtgtataagagacagagctgtgcagcaagcggt

SEQ ID NO: 49 - DNA, 378 bp, Artificial Sequence
  Synthetic
atggaagttcaacttgtagagagcggaggtggctcagtccagccagggggatcgctcacacttagttgcggtacttccggacgaacgttcaatgttatggggtggtttcgtcaagcacctggaaaggagcgggaatttgtcgccgctgtacggtggtcatctactggaatatattacacgcaatacgcagatagcgttaaatcgcgatttactatcagtcgggataatgccaagaacactgtatatctggaaatgaacagcctgaaaccggaagataccgcggtgtattattgcgctgcagatacttataattcaaacccagctagatgggatggatatgatttttggggccagggcacccaggtcaccgtgagcagc

SEQ ID NO: 50 - DNA, 369 bp, Artificial Sequence
  Synthetic
atggaagttcaacttgtagagagcggaggtgagcttgtacaagcaggtggatcacttagactatcttgcgcagcttccgggctcacatttagttcgtacaatatggggtggttccgtagggcaccaggtaaggagcgtgaatttgtcgcaagtataacgtggtcaggacgtgacactttttacgcggattccgtaaaagggcgatttacgatcagtcgtgataacgctaagaatacggtctatcttcaaatgtcaagtctaaaacctgaagataccgcggtgtattattgcgcagctaatccatggcctgtcgccgcaccaagaagcggtacgtattggggccagggcacccaggtaacagtgagcagc

SEQ ID NO: 51 - DNA, 360 bp, Artificial Sequence
  Synthetic
atgcaggtccagctccaagagtctggaggtggttctgtccaaccaggaggttcactacgtctaagctgcgcagcttccggtttcaccttcgacgattatgcgatgtcttgggtacgccaggttcctggaaagggattagagtgggtctcgaccatcaactggaacggaggttctgcagaatatgcagagcctgtcaaaggacgtttcacaatttcgcgggacaacgctaaaaatactgtatatttacagatgaatagtttgaagctggaagataccgcggtgtattattgcgccaaagatgcggacctggtatggtacaacctgtcaaccggacaaggaacacaagtaacggtgtcgagc

SEQ ID NO: 52 - DNA, 16 bp, Artificial Sequence
  Synthetic
acgtacgttgcatgca

SEQ ID NO: 53 - DNA, 34 bp, Artificial Sequence
  Synthetic
gataccgcggtgtattattgcgcaaaggacgcgg

SEQ ID NO: 54 - DNA, 34 bp, Artificial Sequence
  Synthetic
gataccgcggtgtattattgcgctaaagacgcgg

SEQ ID NO: 55 - DNA, 34 bp, Artificial Sequence
  Synthetic
cagctcttcctgcgctgcttaccgtaacttgtgt

SEQ ID NO: 56 - DNA, 34 bp, Artificial Sequence
  Synthetic
cagctcttcctgcgctgcttacagtgacctgtgt

SEQ ID NO: 57 - DNA, 34 bp, Artificial Sequence
  Synthetic
gataccgcggtgtattattgcgccaaagatgcgg

SEQ ID NO: 58 - DNA, 34 bp, Artificial Sequence
  Synthetic
gataccgcggtgtattattgcgctgcagatactt

SEQ ID NO: 59 - DNA, 34 bp, Artificial Sequence
  Synthetic
gataccgcggtgtattattgcgcagctaatccat

SEQ ID NO: 60 - DNA, 34 bp, Artificial Sequence
  Synthetic
gataccgcggtgtattattgcaatgtcaacgtgg

SEQ ID NO: 61 - DNA, 36 bp, Artificial Sequence
  Synthetic
gctgtctcttatacacatctgacgctgccgacgagc

SEQ ID NO: 62 - protein, 7 aa, Artificial Sequence
  Synthetic
NIFRRDS

SEQ ID NO: 63 - protein, 7 aa, Artificial Sequence
  Synthetic
TIFGSVS

SEQ ID NO: 64 - protein, 7 aa, Artificial Sequence
  Synthetic
YIFDGGR

SEQ ID NO: 65 - protein, 7 aa, Artificial Sequence
  Synthetic
NISYSLP

SEQ ID NO: 66 - protein, 7 aa, Artificial Sequence
  Synthetic
YISSLAP

SEQ ID NO: 67 - protein, 12 aa, Artificial Sequence
  Synthetic
LVAAITRGGNTY

SEQ ID NO: 68 - protein, 12 aa, Artificial Sequence
  Synthetic
FVAAINRGSNTY

SEQ ID NO: 69 - protein, 12 aa, Artificial Sequence
  Synthetic
FVASIDYGSSTN

SEQ ID NO: 70 - protein, 12 aa, Artificial Sequence
  Synthetic
FVAAIASGGNTN

SEQ ID NO: 71 - protein, 12 aa, Artificial Sequence
  Synthetic
FVAGINSGGTTN

SEQ ID NO: 72 - protein, 10 aa, Artificial Sequence
  Synthetic
VSSARQQYFW

SEQ ID NO: 73 - protein, 10 aa, Artificial Sequence
  Synthetic
ARILGDGSFA

SEQ ID NO: 74 - protein, 14 aa, Artificial Sequence
  Synthetic
AKRTILYTRGDWYP

SEQ ID NO: 75 - protein, 10 aa, Artificial Sequence
  Synthetic
AADTRASQFG

SEQ ID NO: 76 - protein, 10 aa, Artificial Sequence
  Synthetic
VAARSRDKYG

SEQ ID NO: 77 - DNA, 59 bp, Artificial Sequence
  Synthetic
  misc_feature (30)..(45): n is a, c, g, or t
aatgatacggcgaccaccgagatctacacnnnnnnnnnnnnnnnntcgtcggcagcgtc

SEQ ID NO: 78 - DNA, 33 bp, Artificial Sequence
  Synthetic
ctgtctcttatacacatctgacgctgccgacga

SEQ ID NO: 79 - DNA, 33 bp, Artificial Sequence
  Synthetic
tcgtcggcagcgtcagatgtgtataagagacag

SEQ ID NO: 80 - protein, 6 aa, Artificial Sequence
  Synthetic
LSLIHI

SEQ ID NO: 81 - DNA, 36 bp, Artificial Sequence; identical to SEQ ID NO: 61
  Synthetic

SEQ ID NO: 82 - DNA, 36 bp, Artificial Sequence
  Synthetic
gctcgtcggcagcgtcagatgtgtataagagacagc

SEQ ID NO: 83 - protein, 12 aa, Artificial Sequence
  Synthetic
AVSYTHLTLPTS

SEQ ID NO: 84 - DNA, 51 bp, Artificial Sequence; identical to SEQ ID NO: 15
  Synthetic

SEQ ID NO: 85 - DNA, 33 bp, Artificial Sequence; identical to SEQ ID NO: 78
  Synthetic

SEQ ID NO: 86 - protein, 10 aa, Artificial Sequence
  Synthetic
SSASDVYKRQ

SEQ ID NO: 87 - protein, 10 aa, Artificial Sequence
  Synthetic
  MISC_FEATURE (1)..(1): X is A or V
  MISC_FEATURE (2)..(8): X is any amino acid
  MISC_FEATURE (9)..(9): X is H, L, F, or Y
  MISC_FEATURE (10)..(10): X is any amino acid
XXXXXXXXXX

SEQ ID NO: 88 - protein, 14 aa, Artificial Sequence
  Synthetic
  MISC_FEATURE (1)..(1): X is A or V
  MISC_FEATURE (2)..(12): X is any amino acid
  MISC_FEATURE (13)..(13): X is H, L, F, or Y
  MISC_FEATURE (14)..(14): X is any amino acid
XXXXXXXXXXXXXX

SEQ ID NO: 89 - protein, 18 aa, Artificial Sequence
  Synthetic
  MISC_FEATURE (1)..(1): X is A or V
  MISC_FEATURE (2)..(16): X is any amino acid
  MISC_FEATURE (17)..(17): X is H, L, F, or Y
  MISC_FEATURE (18)..(18): X is any amino acid
XXXXXXXXXXXXXXXXXX

SEQ ID NO: 90 - protein, 7 aa, Artificial Sequence
  Synthetic
  MISC_FEATURE (1)..(1): X is S, T, N, or Y
  MISC_FEATURE (3)..(3): X is S or F
  MISC_FEATURE (4)..(7): X is any amino acid
XIXXXXX

SEQ ID NO: 91 - protein, 12 aa, Artificial Sequence
  Synthetic
  MISC_FEATURE (1)..(1): X is L or F
  MISC_FEATURE (4)..(4): X is G, S, T, or A
  MISC_FEATURE (6)..(6): X is G, S, T, A, D, or N
  MISC_FEATURE (7)..(7): X is any amino acid
  MISC_FEATURE (9)..(9): X is G, S, T, or A
  MISC_FEATURE (10)..(10): X is S, T, I, or N
  MISC_FEATURE (12)..(12): X is N or Y
XVAXIXXGXXTX

SEQ ID NO: 92 - protein, 7 aa, Artificial Sequence
  Synthetic
NIFTYGY

SEQ ID NO: 93 - protein, 7 aa, Artificial Sequence
  Synthetic
TIFWDEH

SEQ ID NO: 94 - protein, 7 aa, Artificial Sequence
  Synthetic
SIFQPVD

SEQ ID NO: 95 - protein, 7 aa, Artificial Sequence
  Synthetic
TIFVWLY

SEQ ID NO: 96 - protein, 7 aa, Artificial Sequence
  Synthetic
TISYKGD

SEQ ID NO: 97 - protein, 7 aa, Artificial Sequence
  Synthetic
NIFGNTD

SEQ ID NO: 98 - protein, 7 aa, Artificial Sequence
  Synthetic
YISYYNS

SEQ ID NO: 99 - protein, 7 aa, Artificial Sequence
  Synthetic
YISVWQG

SEQ ID NO: 100 - protein, 7 aa, Artificial Sequence
  Synthetic
TIFYGSY

SEQ ID NO: 101 - protein, 7 aa, Artificial Sequence
  Synthetic
SISPWYA

SEQ ID NO: 102 - protein, 7 aa, Artificial Sequence
  Synthetic
SISAWDV

SEQ ID NO: 103 - protein, 7 aa, Artificial Sequence
  Synthetic
YISDRGT

SEQ ID NO: 104 - protein, 12 aa, Artificial Sequence
  Synthetic
LVATIDYGTNTY

SEQ ID NO: 105 - protein, 12 aa, Artificial Sequence
  Synthetic
FVATIDDGGSTY

SEQ ID NO: 106 - protein, 12 aa, Artificial Sequence
  Synthetic
LVASIGWGGSTN

SEQ ID NO: 107 - protein, 12 aa, Artificial Sequence
  Synthetic
LVATITGGGSTN

SEQ ID NO: 108 - protein, 12 aa, Artificial Sequence
  Synthetic
FVASINRGGITN

SEQ ID NO: 109 - protein, 12 aa, Artificial Sequence
  Synthetic
LVAGIGAGGITN

SEQ ID NO: 110 - protein, 12 aa, Artificial Sequence
  Synthetic
FVATIDTGSITY

SEQ ID NO: 111 - protein, 12 aa, Artificial Sequence
  Synthetic
LVAAISYGGTTY

SEQ ID NO: 112 - protein, 12 aa, Artificial Sequence
  Synthetic
FVAAIAEGGSTY

SEQ ID NO: 113 - protein, 12 aa, Artificial Sequence
  Synthetic
LVAGINGGGNTY

SEQ ID NO: 114 - protein, 12 aa, Artificial Sequence
  Synthetic
FVATITRGTNTY

SEQ ID NO: 115 - protein, 12 aa, Artificial Sequence
  Synthetic
FVAAIGAGSITY

SEQ ID NO: 116 - protein, 10 aa, Artificial Sequence
  Synthetic
VGYGGGSLYT

SEQ ID NO: 117 - protein, 14 aa, Artificial Sequence
  Synthetic
VSFSRHTDYSDRLN

SEQ ID NO: 118 - protein, 18 aa, Artificial Sequence
  Synthetic
VLGYDSNTRDTGYYKWHG

SEQ ID NO: 119 - protein, 14 aa, Artificial Sequence
  Synthetic
VGNTPDFSDEGQFW

SEQ ID NO: 120 - protein, 14 aa, Artificial Sequence
  Synthetic
VGYSYYQASSKGLR

SEQ ID NO: 121 - protein, 18 aa, Artificial Sequence
  Synthetic
VDYVSQQYYSSYTHAVFT

SEQ ID NO: 122 - protein, 18 aa, Artificial Sequence
  Synthetic
VVVSTSIHDGKDKDFQLQ

SEQ ID NO: 123 - protein, 11 aa, Artificial Sequence
  Synthetic
AITILAAWLFY

SEQ ID NO: 124 - protein, 18 aa, Artificial Sequence
  Synthetic
VVIEWEGYGPRLYLGAFL

SEQ ID NO: 125 - protein, 14 aa, Artificial Sequence
  Synthetic
AQGLPPESSVAYLA

SEQ ID NO: 126 - protein, 10 aa, Artificial Sequence
  Synthetic
AYWTDYQYYG

SEQ ID NO: 127 - protein, 18 aa, Artificial Sequence
  Synthetic
VPELYYGGAWNFYSTPFV

(19) United States
(12) Patent Application Publication
(10) Pub. No.: US 2022/0090089 A1
(43) Pub. Date: Mar. 24, 2022

(54) ENGINEERED MULTI-OMIC DISPLAY CONSTRUCTS AND DISPLAY SYSTEMS

(71) Applicants: The Broad Institute, Inc., Cambridge, MA (US); Massachusetts Institute of Technology, Cambridge, MA (US)

(72) Inventors: Aviv Regev, Cambridge, MA (US); Evgenij Fiskin, Cambridge, MA (US)

(21) Appl. No.: 17/484,067

(22) Filed: Sep. 24, 2021

Related U.S. Application Data
(60) Provisional application No. 63/082,560, filed on Sep. 24, 2020.

(51) Int. Cl. C12N 15/63; C12N 15/10

(52) U.S. Cl. CPC C12N 15/63; C12N 15/1037

(57) ABSTRACT

Described in several embodiments herein are engineered phagemids and bacteriophages containing the same. Also described in several embodiments herein are methods of using the engineered phagemids and bacteriophages containing the same. In some embodiments, the engineered phagemids and bacteriophages containing the same are capable of providing multi-omic information at the single-cell level.

### CROSS-REFERENCE TO RELATED APPLICATIONS

This application claims the benefit of U.S. Provisional Application No. 63/082,560, filed Sep. 24, 2020. The entire contents of the above-identified applications are hereby fully incorporated herein by reference.

### STATEMENT REGARDING FEDERALLY SPONSORED RESEARCH

This invention was made with government support under Grant No. HG006193 awarded by National Institutes of Health. The government has certain rights in the invention.

### REFERENCE TO AN ELECTRONIC SEQUENCE LISTING

The contents of the electronic sequence listing “BROD-5195US_ST25.txt”, size is 28,862 bytes (33 KB on disk) and it was created on Sep. 20, 2021, is herein incorporated by reference in its entirety.

### TECHNICAL FIELD

The subject matter disclosed herein is generally directed to engineered phagemids and bacteriophages and uses thereof, particularly in multi-omic analysis.

### BACKGROUND

Massively-parallel single-cell sequencing has become an invaluable tool for the characterization cells by their transcriptome or epigenome, deciphering gene regulation mechanisms, and dissecting cellular ecosystems in complex tissues. Recent work has further demonstrated the additional assessment of proteins in multimodal single-cell assays. In particular, recent advances have highlighted the power of multimodal single-cell assays, such as cellular indexing of transcriptomes and epitopes by sequencing (CITE-seq), that profile both transcriptome and proteins by DNA-barcoded antibodies. The vast combinatorial space of oligonucleotide barcodes thereby theoretically allows parallel quantification of an unrestricted number of epitopes. In practice, however, these approaches are limited by availability of antigen-specific antibodies and costs. Further, as each antibody necessitates separate conjugation with a unique oligonucleotide (oligo)-barcode, the scalable and pooled construction of barcoded antibody libraries is not possible. Moreover, technologies for the combined high-throughput measurement of the epigenome and proteome have not been described.

As such there exists a need for improved compositions, methods, and techniques suitable for multi-omic analysis.

Citation or identification of any document in this application is not an admission that such a document is available as prior art to the present invention.

### SUMMARY

Described in certain embodiments herein are engineered display constructs comprising optionally, a genetically encoded display molecule, a genetically encoded display molecule linker, or both; a genetically encoded affinity molecule; and a genetically encoded sequencing molecule

, wherein the genetically encoded sequencing molecule is fused to or operatively coupled to the genetically encoded affinity molecule and the genetically encoded display molecule.

In certain example embodiments, the sequencing molecule is a barcode polynucleotide, an index polynucleotide, a primer-binding site, an adapter polynucleotide, or any combination thereof. In certain example embodiments, the engineered display construct is a viral vector, a non-viral vector, or a naked polynucleotide, or a system thereof.

In certain example embodiments, the engineered display construct is an expression vector.

In certain example embodiments, the engineered display construct is a prokaryotic cell expression vector or a eukaryotic cell expression vector.

In certain example embodiments, the engineered display construct is a phagemid.

In certain example embodiments, the genetically encoded display molecule is a genetically encoded capsid polypeptide, a genetically encoded prokaryotic cell surface polypeptide, a genetically encoded eukaryotic cell surface polypeptide, a genetically encoded P2A endonuclease polypeptide, or a genetically encoded RepA polypeptide.

Described in certain example embodiments herein are engineered display systems comprising the engineered display construct of any one of the preceding paragraphs.

In certain example embodiments, the display system is an engineered viral display system, an engineered prokaryotic cell display system, an engineered eukaryotic cell display system, an engineered mRNA display system, an engineered ribosome display system, or an engineered DNA display system.

In certain example embodiments, the engineered display system is an engineered bacteriophage; an engineered non-bacteria virus; an engineered bacterial cell; an engineered yeast cell; an engineered mammalian cell; an engineered insect cell; an engineered DNA display system; an engineered ribosome display system; an engineered covalent display system; or an engineered CIS display system.

In certain example embodiments, the engineered display system further comprises a display molecule; an affinity molecule; and a sequencing polypeptide, wherein the sequencing polypeptide is fused to or operatively coupled to the display molecule, the affinity polypeptide, or both.

In certain example embodiments, the display molecule comprises a capsid polypeptide, a yeast cell surface polypeptide, a bacteria cell surface polypeptide, a mammalian cell surface polypeptide, an insect cell surface polypeptide, a puromycin, a ribosome or component thereof, a P2A endonuclease polypeptide, or a RepA polypeptide,

In certain example embodiments, the affinity molecule comprises a peptide, polypeptide, polynucleotide, a small molecule, or any combination thereof.

In certain example embodiments, the affinity molecule is an antibody or fragment thereof.

In certain example embodiments, wherein the affinity molecule comprises or consists of a human or humanized antibody VH domain.

In certain example embodiments, the display system is a bacteriophage.

In certain example embodiments, the display molecule is a capsid polypeptide.

In certain example embodiments, the display molecule is a major capsid polypeptide or a minor capsid polypeptide.

Described in certain embodiments herein are display construct libraries comprising: a plurality of engineered display constructs according to any one of the preceding paragraphs.

In certain example embodiments, the display constructs are engineered phagemids.

In certain example embodiments, two or more engineered display constructs comprise a unique genetically encoded affinity molecule, a unique genetically encoded display molecule, a unique genetically encoded sequencing molecule, or any combination thereof.

In certain example embodiments, each of the engineered display constructs comprise a unique genetically encoded affinity molecule, a unique genetically encoded display molecule, a unique genetically encoded sequencing molecule, or any combination thereof.

Described in certain example embodiments herein are pluralities of engineered display constructs comprising an engineered display construct library as in any one of the preceding paragraphs.

Described in certain example embodiments herein are engineered display system libraries comprising a plurality of engineered display systems described in any of the preceding paragraphs.

In certain example embodiments, the plurality of engineered display systems comprise a plurality of engineered bacteriophages.

In certain example embodiments, two or more engineered display systems comprise a unique affinity molecule, a unique display molecule, a unique sequencing polypeptide, or any combination thereof.

In certain example embodiments, each of the display systems comprise a unique affinity molecule, a unique display molecule, a unique sequencing polypeptide, or any combination thereof.

Described in certain example embodiments herein are methods of multi-omic single cell or single nuclei analysis, comprising: specifically binding one or more individual cells, individual nuclei, or both with an engineered display system or plurality thereof of as in any one of the preceding paragraphs; allowing each affinity molecule to specifically bind a target molecule present inside of and/or on the surface of the one or more individual cells and/or individual nuclei; fixing the specifically bound engineered display system(s) to the one or more individual cells and/or individual nuclei; accessing cellular polynucleotides within one or more individual specifically bound cells and/or individual specifically bound nuclei; accessing the engineered display construct(s) in the specifically bound engineered display construct(s); and characterizing one or more features of the one or more individual specifically bound cells and/or individual specifically bound nuclei based, at least in part, on sequencing, in whole or in part, (i) the accessed genetically encoded affinity molecule, genetically encoded sequencing molecule, or both present in the specifically bound engineered display construct and (ii) the one or more accessed cellular and/or nuclear polynucleotides.

In certain example embodiments, the method further comprises generating, within one or more individual specifically bound cells and/or nuclei, cDNA copies of cellular RNA molecules.

In certain example embodiments, characterizing one or more features is based, at least in part, on sequencing the cDNA copies of cellular RNA molecules.

In certain example embodiments, sequencing comprises sequencing a portion of the accessed genetically encoded affinity molecule, genetically encoded sequencing molecule, or both present in the specifically bound engineered display construct and a portion of each of the one or more accessed cellular and/or nuclear polynucleotides.

In certain example embodiments, the step of accessing polynucleotides present inside the individual cell and/or individual nuclei comprises permeabilizing the cell, permeabilizing the nucleus, lysing the cells, lysing the nucleus or any combination thereof.

In certain example embodiments, the method further comprises tagmenting, within individual cells and/or individual nuclei, genomic DNA to produced tagmented genomic DNA fragments.

In certain example embodiments, sequencing comprises sequencing the one or more tagmented genomic DNA fragments or a portion thereof.

In certain example embodiments, the method further comprises incorporating a cell or nuclei barcode into the one or more cellular polynucleotides, cDNA copies, tagmented genomic DNA fragments, the genetically encoded affinity molecule, the genetically encoded sequencing molecule, or any combination thereof, such that the one or more cellular polynucleotides, cDNA copies, tagmented genomic DNA fragments, genetically encoded affinity molecule, the genetically encoded sequencing molecule, or any combination thereof from the same cell receive the same unique cell and/or from the same nuclei receive the same nuclei barcode sequence.

In certain example embodiments, the method further comprises incorporating into the one or more cellular polynucleotides, cDNA copies, tagmented genomic DNA fragments, the genetically encoded affinity molecule, the genetically encoded sequencing molecule, or any combination thereof, one or more barcodes; one or more PCR handles; one or more unique molecular identifiers (UMIs); one or more affinity tags; one or more sequencing adapters; one or more linkers; a poly(T) sequence; a poly(A) sequence; one or more primer sites; or any combination thereof.

In certain example embodiments, the method further comprises amplifying the one or more cellular polynucleotides, nuclear polynucleotides, cDNA copies, tagmented genomic DNA fragments, the genetically encoded affinity molecule, the genetically encoded sequencing molecule, or any combination thereof.

In certain example embodiments, the method further comprises mixing the one or more cellular polynucleotides, cDNA copies, tagmented genomic DNA fragments, the genetically encoded affinity molecule, the genetically encoded sequencing molecule, or any combination thereof with an oligonucleotide-adorned bead, wherein each oligonucleotide on the oligonucleotide-adorned bead comprises one or more linkers; one or more barcodes; one or more unique molecular identifiers (UMIs); one or more affinity tags; one or more sequencing adapters; one or more reaction handles or substrates; one or more primer sites; a poly(T) sequence; a poly(A) sequence; one or more PCR handles; or any combination thereof.

In certain example embodiments, the method further comprises isolating a cell and/or nucleus that is specifically bound to and fixed to one or more engineered bacteriophages in or on a substrate, in an individual discrete volume, or container.

In certain example embodiments, the substrate or individual discrete volume is a liquid, a solid, a semi-solid, or a gel.

In certain example embodiments, the substrate or individual discrete volume is a droplet or a slide.

In certain example embodiments, the container is a well, microwell, capillary, or microcapillary.

In certain example embodiments, mixing with an oligonucleotide-adorned bead occurs in or on the substrate or container.

In certain example embodiments, one or more oligonucleotide-adorned beads are present on a surface of the substrate or container and are arranged in an ordered array, wherein each oligonucleotide-adorned bead has a unique barcode corresponding to the x,y coordinate of the oligonucleotide-adorned bead in the array.

In certain example embodiments, the method further comprises depositing a tissue section comprising the one or more individual cells on the ordered array.

In certain example embodiments, the one or more individual cells are present in a tissue sample and specific binding and fixing occurs in situ.

In certain example embodiments, sequencing the genetically encoded affinity molecule, the genetically encoded sequencing molecule, or both and sequencing the one or more cellular polynucleotides, one or more nuclear polynucleotides, or both occurs in situ.

In certain example embodiments, the method further comprises converting unmethylated cytosines to uracil in the genomic DNA via bisulfite conversion prior to sequencing the genomic DNA or portion thereof.

In certain example embodiments, the one or more features comprise a cellular RNA expression profile; a surface protein expression profile; an epigenetic feature of a genomic DNA region in the cell; or any combination thereof.

In certain example embodiments, the epigenetic feature comprises a profile of chromatin accessibility along the genomic DNA region; a DNA binding protein occupancy for a binding site in the genomic DNA region; a nucleosome-free DNA in the genomic DNA region; a positioning of the nucleosomes along the genomic DNA region; methylation status; chromatin states; or any combination thereof.

In certain example embodiments, sequencing comprises a single cell, single nucleus sequencing technique, or both.

Described in certain example embodiments herein are methods of diagnosing, monitoring, or prognosing a condition or disease in a subject, comprising characterizing a feature of one or more individual cells in the subject at one or more time points using a method as in any one of the preceding paragraphs; and providing a diagnosis, prognosis, or condition or disease status based on the feature.

Described in certain embodiments herein are methods of generating a specific pool of engineered display constructs or engineered display systems having a desired target affinity, comprising (a) generating an input display construct or engineered display system library, wherein each display construct or display system present in the input library is as in any one of the preceding paragraphs and elsewhere herein; (b) removing from the input library via negative selection at least some of the engineered display constructs or engineered display systems in the input library that do not specifically bind or otherwise associate with a desired target; (c) positively selecting engineered display constructs or engineered display systems form the pool formed after step (b) that specifically bind or otherwise associate with the desired target; and (d) amplifying the positively selected engineered display constructs or engineered display systems.

In certain example embodiments, the method further comprises repeating steps (b) through (c) or through (d) one or more times, wherein the input for step (b) is the output from step (c) or step (d).

In certain example embodiments, the method further comprises sequencing one or more regions of the positively selected engineered display constructs.

Described in certain embodiments herein are kits for performing multi-omic single cell analysis, comprising an engineered display construct, an engineered display construct library, and/or an engineered display system or plurality thereof as in of any one of the preceding paragraphs.

In certain example embodiments, the affinity molecule of each engineered display system is capable of specifically binding a predetermined target present on the surface of and/or inside of a cell and/or nucleus.

In certain example embodiments, the genetically encoded affinity molecule is capable of generating an affinity molecule polypeptide capable of specifically binding a predetermined target present on the surface of and/or inside of a cell and/or nucleus.

In certain example embodiments, the predetermined target is a microorganism protein; a cancer-associated protein; an immune checkpoint inhibitor; a cell-type marker; a cell-state marker; a non-cancer disease or condition biomarker; or any combination thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1N—PHAGE-ATAC for massively-parallel simultaneous measurement of protein epitopes and chromatin accessibility. (FIG. 1A) Schematic of engineered nanobody-displaying M13 phages used for PHAGE-ATAC. Nanobodies are displayed via fusion to p3, the PAC-tag is placed in the linker between nanobody and p3. M13 phagemids contain a pelB leader for periplasmic secretion and incorporation of fusions during phage assembly. (FIG. 1B) (SEQ ID NO: 61) The PAC-tag RD1 sequence allows for capture by 10×ATAC gel bead oligos (shown in FIG. 4A), without interrupting translation (right). (FIG. 1C) Schematic of PHAGE-ATAC workflow. After phage nanobody staining, fixation, lysis and tagmentation in bulk (leftmost), single cells and 10×ATAC gel beads are encapsulated into droplets using 10× microfluidics followed by linear amplification with simultaneous droplet barcoding of chromatin fragments and phagemids via hybridization of 10× barcoding primers to RD1 sequences (second from left). Separate PDT and ATAC sequencing libraries are prepared (shown in FIG. 5). Representative BioAnalzyer traces of libraries are shown (right). BC, 10× bead barcode. (FIGS. 1D-1K) Single-cell ATAC and EGFP specificity in a species-mixing experiment. (FIG. 1D) Experimental scheme. (FIG. 1E) Number of human (x axis) and mouse (y axis) ATAC fragments associated with each bead barcode (dots), shaded by assignment as human EGFP+ (light blue), human EGFP− (dark blue as represented in greyscale), mouse (red as represented in greyscale), doublet (purple as represented in greyscale, >10% human and mouse fragments). (FIG. 1F) EGFP PDT counts (y axis, log₁₀ scale) and number of ATAC fragments (x axis, log₁₀ scale) for each bead barcode (dots) shaded as in (FIG. 1E) (greyscale legend). (FIGS. 1G-1H) Distributions of EGFP PDTs (G, y axis, log₁₀ scale) and ATAC fragments (H, y axis, log₁₀ scale) in each of the three populations (x axis) (Mann-Whitney one-tailed, ***p<10⁻⁴, NS=not significant). Line: median. (FIGS. 1I-1K) PDT quantification is consistent with flow cytometry. EGFP fluorescence (FIG. 1I, y axis) and distribution (FIG. 1J, x axis) and distribution of EGFP PDT (FIG. 1K, x axis) in EGFP+ (light blue as represented in greyscale) and EGFP− (dark blue as represented in greyscale) human cells. (FIGS. 1L-1N) PHAGE-ATAC and CITE-seq compare well in human PBMCs. (FIGS. 1L and 1M) Two-dimensional joint embedding of scRNA-seq profiles from PBMCs from published CITE-seq (Stoeckius et al., 2017) and of scATAC-seq profiles from PBMCs generated by PHAGE-ATAC, colored by annotated cell types (FIG. 1L) or by the level of protein marker ADTs (M, top) or PDTs (M, bottom). (FIG. 1N) Agreement between protein level estimates from CITE-seq and PHAGE-ATAC. ADT (y axis, centered log ratio (CLR)) and PDT (x axis, CLR) for each marker gene across cell types (dots, shaded as in FIG. 1L), Pearson's r is shown.

FIGS. 2A-2M—PHAGE-ATAC compatible phage nanobodies enable sample multiplexing and can be selected using phage display. (FIG. 2A) Generation of phage hashtags by silent mutations. Shown is a schematic for four anti-CD8 phage hashtags and a subsequent hashing experiment using CD8 T cells from four human donors. (FIGS. 2B-2H) Effective demultiplexing of phage hashtags. (FIG. 2B) PDT counts (greyscale bar, CLR) for each hashtag (rows) across cells (columns) sorted by their HTODemux classification (Phage hash ID). (FIG. 2C) PDT count distributions for each hashtag (colored histograms) across the four Phage hash IDs (Wilcoxon two-tailed, ***p<10⁻⁴). (FIG. 2D) Two-dimensional embedding of cell barcodes by PDT count data, colored by PDT count for the marked hashtag (4 left panels) or by singlet/doublet classification (right). (FIGS. 2E-2F) Distribution of the number of ATAC fragments per barcode (FIG. 2E, y axis) or PDT counts (FIG. 2F, y axis) in cell barcodes in each category (x axis) (Mann-Whitney two-tailed, ***p<10′, NS=not significant). Line: median. (FIG. 2G) Number and percent (color) of barcodes shared between each genotype-based (Genotype ID, rows) and Phage hashtag ID-based (columns) assignments. Top: overall accuracy. (FIG. 2H) Proportion of cells of each type (y axis) within each assigned barcode category (x axis) based on either genotype (left) or and hashtags (right), and in the negative fraction (far right). (FIGS. 2I-2M), Selection of PHAGE-ATAC nanobodies by phage display. (FIG. 2I) Schematic of phage display selection using PANL (see Methods in Working Examples herein). PANL is panned against EGFP-expressing cells (HEK293T-EGFP-GPI) with preceding counter-selection against antigen-devoid parental cells (HEK293T). Bound phages are eluted, used to infect bacterial hosts and output libraries are generated. After multiple selection rounds, antigen-recognizing phage nanobody clones are picked, phagemids are isolated and nanobody inserts are sequenced. (FIG. 2J) Flow cytometry analysis of selection progress. Flow cytometry plots of EGFP fluorescence (y axis) and phage binding (x axis, AlexaFluor647 area) to EGFP-GPI-expressing HEK293T cells (EGFP^{high} and EGFP^{lo}) in, from left, the input library and after each of three consecutive selection cycles (see also FIG. 6C and Methods). (FIG. 2K) Flow cytometry screen of 94 phage nanobody clones derived from selection round 3. Ratio of Q2 to Q1 signal (as defined in FIG. 1J) when staining EGFP-GPI-expressing HEK293T (EGFP^{hi} and EGFP^{lo}) cells with individual phage nanobodies after the 3^{rd} round of selection. Dashed line: threshold of Q2/Q1=1 used for calling positive clones. (FIG. 2L) (SEQ ID NO: 62-76) CDR sequences and CDR3 length of selected clones obtained by Sanger sequencing. * non-randomized constant positions in PANL library (see also FIG. 14A). (FIG. 2M) Flow cytometry plots of EGFP fluorescence (y axis) and phage binding (x axis, AlexaFluor647 area) to EGFP-GPI-expressing HEK293T cells (EGFP^{hi} and EGFP^{lo}) using an immunization-based (Rothbauer et al., 2006) anti-EGFP Nb-displaying phage (middle), clone C5 from this screen (right) and an anti-mCherry phage negative control (left).

FIGS. 3A-3B—Barcoding strategies for epitope quantification by PHAGE-ATAC and CITE-seq. (FIG. 3A). Nanobody-displaying phages for PHAGE-ATAC. The phagemid contained within a particular phage particle encodes the protein displayed on that same phage, and PHAGE-ATAC leverages the hypervariable nanobody CDR3 sequences as unique genetic barcode identifiers for each phage. (FIG. 3B) Oligonucleotide-conjugated antibodies for CITE-seq. Each antibody is separately conjugated with a unique DNA-barcode.

FIGS. 4A-4C—Phage barcode amplification using 10× Genomics scATAC-seq primers enabled by a modified Illumina Read 1 (RD1) sequence. (FIG. 4A) (SEQ ID NO: 77) Schematic of gel bead oligos showing Illumina P5 sequence (P5), random bead barcode (BC) and the first 14 bp of RD1 used for hybridization with RD1-containing chromatin fragments and engineered PHAGE-ATAC phagemids. (FIG. 4B) (SEQ ID NO: 78-86) Nanobody-encoding phagemid constructs for RD1-mediated CDR3 barcode capture by 10× Genomics primers. The top strand is the coding strand. Orientation (arrows and shaded boxes), nucleotide sequence and translation product of RD1-containing constructs are shown. To avoid generating a stop codon by introduction of RD1 into the nanobody-p3 reading frame additional codons are introduced to maintain the reading frame across RD1, thus establishing the PAC tag. (FIG. 4C) Agarose gel after two-step PCR consisting of linear amplification using the 10×ATAC primer followed by exponential PCR using P5 and Illumina Read 2 (RD2)-containing nanobody-specific primers. PDTs were only obtained for PAC-tagged phagemids with RD1 located on the non-coding strand (3′-5′ orientation relative to nanobody). Abbreviations as in (FIG. 4A). Control PCR was performed using two primers hybridizing within the nanobody sequence (Methods)).

FIG. 5—Workflow for separate preparation of scATAC and PDT libraries after droplet-based indexing. Schematic of post barcoding steps for the generation of ATAC and PDT sequencing libraries (see Methods in Working Examples herein). After breaking emulsions, barcoded linear amplification products are purified and samples are split. ATAC fragment libraries are immediately processed for sample index PCR. PDT libraries are first amplified in a PDT-specific PCR using a CDR3 flanking constant nanobody sequence as PCR handle. PDT amplification allows RD2 adapter introduction required for final sample indexing. P5 and P7, Illumina P5 and P7 sequences. CBC, random 10× bead cell barcode. i7, sample index.

FIGS. 6A-6G—Detection of membrane-localized EGFP via anti-EGFP nanobody-displaying phages. (FIGS. 6A-6B) Membrane expressed EGFP. (FIG. 6A) Microscopy images of HEK293T cells expressing indicated constructs, showing differential localization of untagged cytosolic EGFP (pCAG-EGFP, middle) and GPI-anchored membrane-localized EGFP (pCAG-EGFP-GPI, right, Methods in Working Examples herein). (FIG. 6B) Schematic of surface-exposed GPI-anchored EGFP. (FIG. 6C) Schematic for detection of phage recognition via flow cytometry. Phage-stained cells are incubated with mouse anti-M13 coat protein antibodies followed by detection by Alexa Fluor 647-conjugated anti-mouse secondary antibodies. Phage binding is thus reflected by Alexa Fluor 647 signal. (FIG. 6D) Flow cytometry analysis of anti-EGFP phage nanobody binding to EGFP-expressing HEK293T cells. EGFP fluorescence (y axis) and phage binding (x axis, Alexa Fluor 647) in each of the HEK293T cell populations as in FIG. 6A, either unstained (left) or stained with an anti-EGFP phage (right). EGFP-expressing cells were always characterized by the presence of both EGFPhi and EGFPlo populations. (FIG. 6E) Specificity of detection. As in FIG. 6D but using the indicated staining controls for specific staining of membrane-EGFP-expressing cells. (FIGS. 6F-6G) PAC-tag does not impact nanobody display and antigen interaction. EGFP fluorescence (FIG. 6F, y axis) and phage binding (FIG. 6F, x axis, Alexa Fluor 647) and distribution of level of phage binding (FIG. 6G) for phage-stained EGFP-GPI expressing cells using indicated phage nanobodies (for RD1 sequences see FIG. 4B).

FIGS. 7A-7B—Optimization of fixation and lysis conditions for PHAGE-ATAC species-mixing experiment. EGFP fluorescence (FIG. 7A, y axis) and phage binding (A, x axis, Alexa Fluor 647) and distribution of level of phage binding (FIG. 7B) for EGFP-GPI expressing cells stained with PAC-tagged anti-EGFP-Nb displaying phages after fixation and permeabilization using indicated conditions.

FIG. 8—Computational workflow for PHAGE-ATAC data analysis. Paired-end sequencing output is demultiplexed using sample index information (left) to recover ATAC and PDT fastqs. ATAC fastqs are processed using CellRanger-ATAC count for fragment alignment, assignment of cell barcodes and generation of peak-cell barcode matrices. CDR3 barcode sequences are used to search PDT_R3 fastqs and identify CDR3-containing sequencing clusters. Matching of cluster identifiers is used to derive corresponding cell barcodes from PDT_R2 fastqs. Recovered PDT cell barcode lists are filtered using cell barcodes called by CellRanger. Cell barcode occurrences are counted to generate PDT-cell barcode count matrices (see also Methods of the Working Examples herein).

FIGS. 9A-9C—PHAGE-ATAC quality metrics for human-mouse species-mixing experiment. (FIG. 9A) Fraction (y axis) and number (x axis, log 10 scale) of unique chromatin fragments overlapping peaks for each barcode (dot) shaded by populations (greyscale legend). (FIGS. 9B-9C) Distribution of fraction of unique ATAC fragments overlapping peaks (FIG. 9B, y axis) or TSS (FIG. 9C, y axis) in each of the three cell populations (x axis) (Mann-Whitney two-tailed, ***p<10-4, NS=not significant). Line: median.

FIGS. 10A-10C—Validation of PAC-tagged anti-CD4, anti-CD8 and anti-CD16 nanobody-displaying phages. (FIG. 10A) Flow cytometry gating strategy for analyzed phage-stained PBMCs. (FIG. 10B) Flow cytometry-based binding assessment of indicated surface marker-recognizing phage nanobodies to gated lymphocyte and monocyte populations, anti-EGFP pNb was used as negative control. (FIG. 10C) Comparison of PBMCs stained with a well-characterized anti-CD4 antibody or generated anti-CD4 phage nanobody. Phage binding is reflected by Alexa Fluor 647 fluorescent signal intensity.

FIGS. 11A-11B—Optimization of fixation and lysis conditions for PHAGE-ATAC using PBMCs. (FIG. 11A) Binding of generated anti-CD4 phage nanobodies to PBMCs under indicated conditions. Two different formaldehyde concentrations as well as various depicted lysis buffers were used. Phage binding is reflected by Alexa Fluor 647 fluorescent signal intensity. (FIG. 11B) Histogram of data in (FIG. 11A).

FIGS. 12A-12E—Multimodal single-cell analysis of human PBMCs using PHAGE-ATAC. (FIG. 12A) Two-dimensional joint embedding of scRNA-seq profiles from PBMCs from published CITE-seq (Stoeckius et al., 2017) and of scATAC-seq profiles from PBMCs generated by PHAGE-ATAC, colored by the measured RNA level from CITE-Seq (top panels) or by gene activity scores from PHAGE-ATAC (bottom panels) (Methods). (FIGS. 12B-12C) PHAGE-ATAC gating by phage staining highlights cell type specific loci. (FIG. 12B) PDT count-based classification of CD4+ and CD8+ T cells. PDT counts (CLR transformed) of CD8 (y axis) and CD4 (x axis) in each cell (dots). Red boxes: gates for CD4+ and CD8+ cells. (FIG. 12C) Average fold change (x axis, loge) and associated significance (y axis, −log₁₀ (P-value) for each gene activity comparing between PDT-classified CD4 and CD8 T cells shown in B. Known bona fide markers of either CD4 or CD8 T cells are marked. (FIG. 12D) Negative control. Embedding of PHAGE-ATAC data as in (FIG. 12A), colored by anti-EGFP pNb PDT. (FIG. 12E) Distribution of phage counts (y axis, log₁₀) for each cell barcode for each assayed nanobody (x axis).

FIGS. 13A-13G—Validation of phage hashtag binding. (FIG. 13A) Flow cytometry of anti-CD8 hashtag phages bound (Alexa Fluor 647 fluorescent signal, x axis) to lymphocytes gated via flow cytometry of phage-stained PBMCs (as shown in FIG. 10A). Phage binding is reflected by Alexa Fluor 647 fluorescent signal intensity (Methods). (FIG. 13B). Concordance between hashtag-based classification of barcodes and identified mtDNA SNPs. Heteroplasmy (allele frequency percentage; greyscale bar) of different mtDNA variants (rows) in each cell (column), labeled by hashtag assignment (vertical top greyscale bar). (FIG. 13C) Cell type identification. Two-dimensional embedding of hashed CD8 T cells analyzed by PHAGE-ATAC, colored by cell type annotation. (FIG. 13D) Pseudobulk chromatin accessibility track plots for CD8, CD3 and MS4A1 (CD20) loci across identified cell types. (FIG. 13E) Embedding as in B with cells colored by CD8 hashtag PDTs. (FIGS. 13fF13G). Distribution of maximal CD8 PDT density (FIG. 13F, y axis) or unique chromatin fragments (FIG. 13G, y axis) for each cell barcode in CD8− (B cell 1 and B cell 2) and CD8+ (non-B cell) cells (x axis) (Mann-Whitney two-tailed, ***p<10-4).

FIGS. 14A-14D—Establishment of PANL, a fully synthetic high-complexity PAC-tagged phage nanobody library. (FIG. 14A) (SEQ ID NO: 87-91). Schematic of PANL library design and library phagemid. CDR3 sequence diversification and nanobody framework (grey) in PANL are based on a previously reported nanobody randomization strategy (McMahon et al., 2018). White box: expected frequency of amino acids at each hypervariable position (denoted by X), adjusted by using a custom randomized primer mix for library generation (see also Methods in Working Examples herein). CDR3 loops contained either 7, 11 or 15 hypervariable positions, resulting in total CDR3 lengths of 10 (short), 14 (medium) or 18 (long) amino acids. Partially randomized positions are depicted as columns, constant positions contain a single amino acid. A deposited structure of anti-EGFP Nb (PDB: 3ogo (Kubala et al., 2010)) with colored CDR3 loops is shown. PANL phagemid is analogous to the one shown in FIG. 1A. (FIG. 14B) Expected (grey) and observed (red) frequencies (x axis) of amino acids at hypervariable positions (y axis) (Methods). (FIG. 14C) Amplification products of phagemid insert-spanning PCR reactions using depicted primers for 25 randomly picked PANL clones. Product sizes due to presence of long, medium or short CDR3 are shown. (FIG. 14D) (SEQ ID NO: 92-127) CDR3 sequences of selected clones from C obtained by Sanger sequencing, CDR3 length is indicated, * non-randomized constant positions in the PANL library.

FIGS. 15A-15B—Flow cytometry-based screen of nanobody-displaying phage clones from selection round 3. Flow cytometry analysis of round 3 phage nanobody clones for binding to EGFP-GPI expressing cells (EGFP^{hi} and EGFP^{lo} populations can be observed) with either strong (FIG. 15A) or weak (FIG. 15B) binders. Phage nanobodies against mCherry were used as a negative control. Phage binding is reflected by Alexa Fluor 647 signal.

FIG. 16—Estimates of cost per reaction for phage nanobodies. Comparison of cost estimates per reaction step and overall for a phage nanobody produced recombinant.ly

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +1-10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Massively-parallel single-cell profiling has become an invaluable tool for the characterization of cells by their transcriptome or epigenome, deciphering gene regulation mechanisms, and dissecting cellular ecosystems in complex tissues (Klein et al., 2015; Lareau et al., 2019; Macosko et al., 2015; Satpathy et al., 2019). In particular, recent advances have highlighted the power of multimodal single-cell assays (Ma et al., 2020), such as cellular indexing of transcriptomes and epitopes by sequencing (CITE-seq), that profile both transcriptome and proteins by DNA-barcoded antibodies (Mimitou et al., 2019; Peterson et al., 2017; Stoeckius et al., 2017).

Although the vast combinatorial space of oligonucleotide barcodes theoretically allows parallel quantification of an unrestricted number of epitopes, in practice, however, it is still limited by the availability of antigen-specific antibodies. Moreover, each antibody must be separately conjugated with a unique oligonucleotide (oligo)-barcode, which currently does not allow a scalable and pooled construction of barcoded antibody libraries. Finally, technologies for the combined high-throughput measurement of the epigenome and proteome have not been described.

With these limitations in mind, exemplary embodiments herein provide compositions, systems, and methods for multimodal single-cell approach for phage-based multiplex protein measurements and chromatin accessibility profiling using e.g., a scATAC-seq genomics approach. Embodiments herein can provide sensitive quantification of epigenome and proteins, captures mtDNA that can be used in various applications such as a native clonal tracer, introduces phages as renewable and cost-effective reagents for high-throughput single-cell epitope profiling, and leverages phage libraries for the selection of antigen-specific, altogether providing an advantageous platform that addresses various limitations of current approaches and greatly expands the scope of the single-cell profiling toolbox.

Certain exemplary embodiments disclosed herein provide engineered phagemids comprising a genetically encoded capsid polypeptide; a genetically encoded affinity molecule; and a genetically encoded sequencing molecule, wherein the genetically encoded sequencing molecule is fused to or operatively coupled to the genetically encoded affinity molecule and the genetically encoded capsid polypeptide.

Certain exemplary embodiments disclosed herein provide engineered bacteriophages that comprise one or more of the engineered phagemids. In certain example embodiments, the engineered bacteriophage further comprises an engineered capsid comprising: a capsid polypeptide; an affinity molecule; and a sequencing molecule polypeptide, wherein the sequencing molecule polypeptide is fused to or operatively coupled to the capsid polypeptide and/or the affinity polypeptide and wherein the affinity polypeptide is expressed on the surface of the engineered capsid.

Certain exemplary embodiments s disclosed herein provide engineered phagemid libraries that include a plurality of engineered phagemids described herein.

Certain exemplary embodiments disclosed herein provide a plurality of engineered bacteriophages that include, either individually or collectively, comprise one or more of the engineered phagemids described herein. In certain example embodiments, a plurality of engineered bacteriophages that include a phagemid library described herein.

Certain exemplary embodiments s disclosed herein provide methods of multi-omic single cell or single nuclei analysis, comprising specifically binding one or more individual cells, individual nuclei, or both with an engineered bacteriophage or plurality thereof of as described in greater detail elsewhere herein; allowing each affinity molecule to specifically bind a target molecule present inside of and/or on the surface of the one or more individual cells and/or individual nuclei; fixing the specifically bound engineered bacteriophage(s) to the one or more individual cells and/or individual nuclei; accessing cellular polynucleotides within one or more individual specifically bound cells and/or individual specifically bound nuclei accessing the engineered phagemid(s) in the specifically bound engineered bacteriophage(s); and characterizing one or more features of the one or more individual specifically bound cells and/or individual specifically bound nucleic based, at least in part, on sequencing, in whole or in part, (i) the accessed genetically encoded affinity molecule, genetically encoded sequencing molecule, or both present in the specifically bound phagemid and (ii) the one or more accessed cellular and/or nuclear polynucleotides.

Certain exemplary embodiments disclosed herein provide methods of diagnosing, monitoring, or prognosing a condition or disease in a subject, comprising: characterizing a feature of one or more individual cells in the subject at one or more time points using a method as in any one of the preceding paragraphs or as described in greater detail elsewhere herein; and providing a diagnosis, prognosis, or condition or disease status based on the feature.

Certain exemplary embodiments disclosed herein provide kits for performing multi-omic single cell analysis, comprising: a phagemid, phagemid library, and/or an engineered bacteriophage or plurality thereof as described elsewhere herein.

Certain exemplary embodiments disclosed herein provide compositions and methods that are capable of multimodal profiling, including single-cell and/or high throughput multimodal analysis, of the genome, epigenome, proteome and combinations thereof. Embodiments disclosed herein can provide a multimodal single-cell approach for phage-based multiplex protein measurements and chromatin accessibility profiling. Embodiments disclosed herein provide a more cost-effective approach of multi-omic analysis.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered Display Constructs and Display Systems

Described in certain embodiments herein are engineered display constructs comprising optionally, a genetically encoded display molecule; a genetically encoded affinity molecule; and a genetically encoded sequencing molecule, wherein the genetically encoded sequencing molecule is fused to or operatively coupled to the genetically encoded affinity molecule and the genetically encoded display molecule.

Embodiments disclosed herein provide engineered phagemids including a genetically encoded capsid polypeptide; a genetically encoded affinity molecule; and a genetically encoded sequencing molecule, wherein the genetically encoded sequencing molecule is fused to or is operatively coupled to the genetically encoded affinity molecule and the genetically encoded capsid polypeptide. Embodiments disclosed herein provide engineered bacteriophages that contain one or more of the engineered phagemids. In certain example embodiments, the engineered bacteriophage further contains an engineered capsid comprising: a capsid polypeptide; an affinity molecule; and a sequencing molecule polypeptide, wherein the sequencing molecule polypeptide is fused to or operatively coupled to the capsid polypeptide and/or the affinity polypeptide and wherein the affinity polypeptide is expressed on the surface of the engineered capsid.

As used herein, “genetically encoded” refers to a polynucleotide or a polypeptide that is encoded by a polynucleotide that is genomic or extragenomic (such as a plasmid). In the context of this application, genomic or extragenomic does not require that the polynucleotide and/or sequence so described is a naturally occurring polynucleotide and/or sequence, the polynucleotide and/or sequence may be engineered or modified from a naturally occurring polynucleotide and/or sequence. As used herein, “encode”, “encoded”, “encoding” and the like refer to principle that DNA can be transcribed into RNA, which can then optionally be translated into amino acid sequences that can form proteins. As used interchangeably herein, “operatively coupled” and “operably coupled” in the context of recombinant or engineered polynucleotide molecules (e.g. DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g., promoters, enhancers, and termination elements), and/or selectable markers in an expression vector. “Operatively coupled” can also refer to an indirect attachment (i.e., not a direct fusion) of two or more polynucleotide sequences or polypeptides to each other via a linking molecule (also referred to herein as a linker). As used herein, “phagemid” refers to a plasmid that contains an origin of replication for double stranded replication as well as origin of replication from a bacteriophage to facilitate single stranded replication and packaging into phage particles. In some embodiments the phagemid is a vector. As used herein, the term “vector” or is used in reference to a vehicle used to introduce an exogenous nucleic acid sequence into a cell. A vector may include a DNA molecule, linear or circular (e.g., plasmids), which includes a segment encoding an RNA and/or polypeptide of interest operatively linked to additional segments that provide for its transcription and optional translation upon introduction into a host cell or host cell organelles. Such additional segments can include promoter and/or terminator sequences, and can also include one or more origins of replication, one or more selectable markers, an enhancer, a polyadenylation signal, etc. Expression vectors are generally derived from yeast or bacterial genomic or plasmid DNA, or viral DNA, or may contain elements of both. Expression vectors can be adapted for expression in prokaryotic or eukaryotic cells. Expression vectors can be adapted for expression in mammalian, fungal, yeast, or plant cells. Expression vectors can be adapted for expression in a specific cell type via the specific regulator or other additional segments that can provide for replication and expression of the vector within a particular cell type.

### Engineered Display Constructs

Described in certain embodiments herein are engineered display constructs comprising: optionally, a genetically encoded display molecule, a genetically encoded display molecule linker, or both; a genetically encoded affinity molecule; and a genetically encoded sequencing molecule, wherein the genetically encoded sequencing molecule is fused to or operatively coupled to the genetically encoded affinity molecule and the genetically encoded display molecule. Table 1 below shows exemplary display systems and their respective display molecules within the context of the present invention. The display molecules are further described elsewhere herein.

TABLE 1

| | |
|---|---|
| Display System | Display Molecule |
| Bacteriophage* | Capsid (coat protein) |
| Non-bacteria Virus* | Capsid |
| Yeast$ | Cell surface molecule (e.g., an |
| | agglutinin or flocculin) |
| Bacteria# | Cell surface molecule (cell |
| | membrane or cell wall) |
| mRNA\hat{ } | Puromycin |
| ribosome | Ribosome or component thereof |
| DNA display** | |
| CDT (aka Covalent | P2A endonuclease |
| display technology)** | |
| CIS Display** | RepA |
| Mammalian cells $ | Cell surface molecule |
| Insect cells$ | Cell surface molecule |

*Virus-based Display System
**DNA-based Display system
\hat{ }RNA-based Display system
$ Eukaryotic Cell-based display system
#Prokaryotic Cell-based display system

Various bacterial cell display systems have been described such as those set forth in Richins R. D., Kaneva I., Mulchandani A., Chen W. Biodegradation of organophosphorus pesticides by surface-expressed organophosphorus hydrolase. Nat. Biotechnol. 1997; 15:984-987); Ravikumar S., Ganesh I., Yoo I.-K., Hong S. H. Construction of a bacterial biosensor for zinc and copper and its application to the development of multifunctional heavy metal adsorption bacteria. Process Biochem. 2012; 47:758-765; Park T. J., Zheng S., Kang Y. J., Lee S. Y. Development of a whole-cell biosensor by cell surface display of a gold-binding polypeptide on the gold surface. FEMS Microbiol. Lett. 2009; 293:141-147; Tang X., Zhang T., Liang B., Han D., Zeng L., Zheng C., Li T., Wei M., Liu A. Sensitive electrochemical microbial biosensor for p-nitrophenylorganophosphates based on electrode modified with cell surface-displayed organophosphorus hydrolase and ordered mesopore carbons. Biosens. Bioelectron. 2014; 60:137-142. doi: 10.1016/j.bios.2014.04.001; Liang B., Li L., Tang X., Lang Q., Wang H., Li F., Shi J., Shen W., Palchetti I., Mascini M. Microbial surface display of glucose dehydrogenase for amperometric glucose biosensor. Biosens. Bioelectron. 2013; 45:19-24. doi: 10.1016/j.bios.2013.01.050; and Liang B., Zhang S., Lang Q., Song J., Han L., Liu A. Amperometric L-glutamate biosensor based on bacterial cell-surface displayed glutamate dehydrogenase. Anal. Chim. Acta. 2015; 884:83-89. doi: 10.1016/j.aca.2015.05.012; Zhang Z., Liu J., Fan J., Wang Z., Li L. Detection of catechol using an electrochemical biosensor based on engineered Escherichia coli cells that surface-display laccase. Anal. Chim. Acta. 2018; 1009:65-72. doi: 10.1016/j.aca.2018.01.008; Park T. J., Zheng S., Kang Y. J., Lee S. Y. Development of a whole-cell biosensor by cell surface display of a gold-binding polypeptide on the gold surface. FEMS Microbiol. Lett. 2009; 293:141-147; Jose J., Chung J.-W., Jeon B.-J., Maas R. M., Nam C.-H., Pyun J.-C. Escherichia coli with autodisplayed Z-domain of protein A for signal amplification of SPR biosensor. Biosens. Bioelectron. 2009; 24:1324-1329; Lee E.-H., Yoo G., Jose J., Kang M.-J., Song S.-M., Pyun J.-C. SPR biosensor based on immobilized E. coli cells with autodisplayed Z-domains. BioChip J. 2012; 6:221-228; Park M., Jose J., Pyun J.-C. SPR biosensor by using E. coli outer membrane layer with autodisplayed Z-domains. Sens. Actuators B Chem. 2011; 154:82-88; Kronqvist N., Löfblom J., Jonsson A., Wernérus H., Ståhl S. A novel affinity protein selection system based on staphylococcal cell surface display and flow cytometry. Protein Eng. Des. Sel. 2008; 21:247-255 and Kronqvist N., Malm M., Göstring L., Gunneriusson E., Nilsson M., Höidén Guthenberg I., Gedda L., Frejd F. Y., Ståhl S., Löfblom J. Combining phage and staphylococcal surface display for generation of ErbB3-specific Affibody molecules. Protein Eng. Des. Sel. 2011; 24:385-396; Desvaux et al. 2006. FEMS Microbiol Lett. 256(1): 1-15; Freudl R, et al. Cell surface exposure of the outer membrane protein OmpA of Escherichia coli K-12. J Mol Biol. 1986; 188(3):491-4; Charbit A, et al. Probing the topology of a bacterial membrane protein by genetic insertion of a foreign epitope; expression at the cell surface. EMBO J. 1986; 5(11):3029-37; Lee S Y, Choi J H, Xu Z. Microbial cell-surface display. Trends Biotechnol. 2003; 21(1):45-52; Strauss A, Gotz F. In vivo immobilization of enzymatically active polypeptides on the cell surface of Staphylococcus carnosus. Mol Microbiol. 1996; 21(3):491-500; Lee J S, et al. Surface-displayed viral antigens on Salmonella carrier vaccine. Nat Biotechnol. 2000; 18(6):645-8, Pseudomonas aeruginosa outer membrane protein OprF as an expression vector for foreign epitopes: the effects of positioning and length on the antigenicity of the epitope. Gene. 1995; 158(1):55-60; Lang H. Outer membrane proteins as surface display systems. Int J Med Microbiol. 2000; 290(7):579-85; Ruppert A, Arnold N, Hobom G. OmpA-FMDV VP1 fusion proteins: production, cell surface exposure and immune responses to the major antigenic domain of foot-and-mouth disease virus. Vaccine. 1994; 12(6):492-8; Xu Z, Lee S Y. Display of polyhistidine peptides on the Escherichia coli cell surface by using outer membrane protein C as an anchoring motif. Appl Environ Microbiol. 1999; 65(11):5142-7; Hogervorst E J, et al. Efficient recognition by rat T cell clones of an epitope of mycobacterial hsp 65 inserted in Escherichia coli outer membrane protein PhoE. Eur J Immunol. 1990; 20(12):2763-8; Sumuelson et al., J. Biotechnol. 2002. 96(2):129-154; Rutherford and Mourez. Microb Cell Fact. 2006. 5:22; Chen at al. Microbial Cell Factories volume 18, Article number: 70 (2019); Lee et al. 2003. Trends Biotechnol. 21(1):45-52); and Park. Sensors. 2020. 20(10):2775 (Particularly at Table 2), which are each incorporated by reference herein as if expressed in their entireties and can be adapted for use with the present invention in view of this disclosure.

In some embodiments, the engineered display system is an engineered bacterial display system. In some embodiments the engineered display system is an engineered gram negative bacterial display system. In some embodiments, the display molecule is outer membrane protein (Omp)A, OmpC, OmpF, LPP-OmpA, Outer membrane pore protein E precursor (PhoE), INP (Tang X., Zhang T., Liang B., Han D., Zeng L., Zheng C., Li T., Wei M., Liu A. Sensitive electrochemical microbial biosensor for p-nitrophenylorganophosphates based on electrode modified with cell surface-displayed organophosphorus hydrolase and ordered mesopore carbons. Biosens. Bioelectron. 2014; 60:137-142. doi: 10.1016/j.bios.2014.04.001; Liang B., Li L., Tang X., Lang Q., Wang H., Li F., Shi J., Shen W., Palchetti I., Mascini M. Microbial surface display of glucose dehydrogenase for amperometric glucose biosensor. Biosens. Bioelectron. 2013; 45:19-24. doi: 10.1016/j.bios.2013.01.050; and Liang B., Zhang S., Lang Q., Song J., Han L., Liu A. Amperometric L-glutamate biosensor based on bacterial cell-surface displayed glutamate dehydrogenase. Anal. Chim. Acta. 2015; 884:83-89. doi: 10.1016/j.aca.2015.05.012), InaQ-N (Zhang Z., Liu J., Fan J., Wang Z., Li L. Detection of catechol using an electrochemical biosensor based on engineered Escherichia coli cells that surface-display laccase. Anal. Chim. Acta. 2018; 1009:65-72. doi: 10.1016/j.aca.2018.01.008), FadL (Park T. J., Zheng S., Kang Y. J., Lee S. Y. Development of a whole-cell biosensor by cell surface display of a gold-binding polypeptide on the gold surface. FEMS Microbiol. Lett. 2009; 293:141-147), or AIDA-I ((Jose J., Chung J.-W., Jeon B.-J., Maas R. M., Nam C.-H., Pyun J.-C. Escherichia coli with autodisplayed Z-domain of protein A for signal amplification of SPR biosensor. Biosens. Bioelectron. 2009; 24:1324-1329; Lee E.-H., Yoo G., Jose J., Kang M.-J., Song S.-M., Pyun J.-C. SPR biosensor based on immobilized E. coli cells with autodisplayed Z-domains. BioChip J. 2012; 6:221-228; Park M., Jose J., Pyun J.-C. SPR biosensor by using E. coli outer membrane layer with autodisplayed Z-domains. Sens. Actuators B Chem. 2011; 154:82-88). In some embodiments the engineered display system is an engineered gram positive bacterial display system. In some embodiments, the display molecule is APB (Kronqvist N., Löfblom J., Jonsson A., Wernérus H., Ståhl S. A novel affinity protein selection system based on staphylococcal cell surface display and flow cytometry. Protein Eng. Des. Sel. 2008; 21:247-255 and Kronqvist N., Malm M., Göstring L., Gunneriusson E., Nilsson M., Höidén Guthenberg I., Gedda L., Frejd F. Y., Ståhl S., Löfblom J. Combining phage and staphylococcal surface display for generation of ErbB3-specific Affibody molecules. Protein Eng. Des. Sel. 2011; 24:385-396), a lipoprotein (Desvaux et al. 2006. FEMS Microbiol Lett. 256(1): 1-15), a YidC homologue (Desvaux et al. 2006. FEMS Microbiol Lett. 256(1): 1-15), LPXTG (a cell wall associated protein) (Desvaux et al. 2006. FEMS Microbiol Lett. 256(1): 1-15); a CWBD (cell wall binding domain) 1 protein (cell wall associated protein) (Desvaux et al. 2006. FEMS Microbiol Lett. 256(1): 1-15), a CWBD2 protein (cell wall associated protein) (Desvaux et al. 2006. FEMS Microbiol Lett. 256(1): 1-15; a LysM protein (cell wall associated protein) (Desvaux et al. 2006. FEMS Microbiol Lett. 256(1): 1-15), a GW protein (cell wall associated protein) (Desvaux et al. 2006. FEMS Microbiol Lett. 256(1): 1-15), or a S-layer homology domain (SLHD) protein (a cell wall associated protein) (Desvaux et al. 2006. FEMS Microbiol Lett. 256(1): 1-15).

Various yeast display systems have been described. See e.g., Boder E T, Wittrup K D. Yeast surface display for screening combinatorial polypeptide libraries. Nat Biotechnol. 1997; 15(6):553-7; Ye K., Shibasaki S., Ueda M., Murai T., Kamasawa N., Osumi M., Shimizu K., Tanaka A. Construction of an engineered yeast with glucose-inducible emission of green fluorescence from the cell surface. Appl. Microbiol. Biotechnol. 2000; 54:90-96; Shibasaki S., Ueda M., Ye K., Shimizu K., Kamasawa N., Osumi M., Tanaka A. Creation of cell surface-engineered yeast that display different fluorescent proteins in response to the glucose concentration. Appl. Microbiol. Biotechnol. 2001; 57:528-533; Shibasaki S., Ninomiya Y., Ueda M., Iwahashi M., Katsuragi T., Tani Y., Harashima S., Tanaka A. Intelligent yeast strains with the ability to self-monitor the concentrations of intra- and extracellular phosphate or ammonium ion by emission of fluorescence from the cell surface. Appl. Microbiol. Biotechnol. 2001; 57:702-707; Shibasaki S., Tanaka A., Ueda M. Development of combinatorial bioengineering using yeast cell surface display—order-made design of cell and protein for bio-monitoring. Biosens. Bioelectron. 2003; 19:123-130; Wang H., Lang Q., Li L., Liang B., Tang X., Kong L., Mascini M., Liu A. Yeast surface displaying glucose oxidase as whole-cell biocatalyst: Construction, characterization, and its electrochemical glucose sensing application. Anal. Chem. 2013; 85:6107-6112; Liang B., Wang G., Yan L., Ren H., Feng R., Xiong Z., Liu A. Functional cell surface displaying of acetylcholinesterase for spectrophotometric sensing organophosphate pesticide. Sens. Actuators B Chem. 2019; 279:483-489; Liang B., Han L. Displaying of acetylcholinesterase mutants on surface of yeast for ultra-trace fluorescence detection of organophosphate pesticides with gold nanoclusters. Biosens. Bioelectron. 2020; 148:111825, which are each incorporated by reference herein as if expressed in their entireties and can be adapted for use with the present invention in view of this disclosure.

In some embodiments, the display system can be based on a yeast display system, including, but not limited to, any one or more of those previously described. In some embodiments the display molecule is a glucanase-extractable protein such as agglutinin (e.g., alpha agglutinin) or flocculin (see e.g., Kondo A, Ueda M Appl Microbiol Biotechnol. 2004. 64(1):28-40; Chen X. Bioengineered. 2017. 8(2):115-119).

Various ribosome display systems have been described. See e.g., Hanes, J.; Plückthun, A. (1997). “In vitro selection and evolution of functional proteins by using ribosome display”; Proc. Natl. Acad. Sci. U.S.A. 94 (10): 4937-42; Lipovsek, D.; Plückthun, A. (2004). “In-vitro protein evolution by ribosome display and mRNA display”. J. Imm. Methods. 290 (1-2): 51-67. He, M.; Taussig, M. (2007). “Eukaryotic ribosome display with in situ DNA recovery”. Nature Methods. 4 (3): 281-288, which are each incorporated by reference herein as if expressed in their entireties and can be adapted for use with the present invention in view of this disclosure. In some embodiments, the encoding polynucleotide of a ribosome display system includes a spacer fused to an encoding polynucleotide (such as that described in connection with the present invention), where the spacer lacks a stop codon. This prevents release factors from binding and triggering the disassembly of the translational complex resulting in the peptidyl tRNA to stay in the ribosomal tunnel and allowing the translated protein (e.g., affinity molecule) to protrude out of the ribosome and fold. What results is a complex of the encoding mRNA, ribosome (a display molecule in the context of the present invention), and protein (e.g., the affinity molecule) which is free to bind to a target. It will be appreciated that in some embodiments that are based on a ribosome display that the display molecule is not present in an encoding construct (e.g., a display construct) but is included in an engineered display system. In some embodiments, the encoding display construct includes one or more genetically encoded ribosome polypeptides or genetically encoded rRNAs. In some embodiments, the display system can be based on a ribosome display system, including, but not limited to, any one or more of those previously described.

Various mRNA display systems have been described. See e.g., Amstutz P, Forrer P, Zahnd C, Plückthun A (2001). “In vitro display technologies: novel developments and applications”. Current Opinion in Biotechnology. 12 (4): 400-5; Liu R, Barrick J E, Szostak J W, Roberts R W (2000). “Optimized synthesis of RNA-protein fusions for in vitro protein selection”. Methods in Enzymology. 318: 268-93; Kurz M, Gu K, Lohse P A (2000). “Psoralen photo-crosslinked mRNA-puromycin conjugates: a novel template for the rapid and facile preparation of mRNA-protein fusions “Nucleic Acids Research. 28 (18): 83e-83; Roberts R W, Szostak J W (1997). “RNA-peptide fusions for the in vitro selection of peptides and proteins”. Proc Natl Acad Sci USA. 94 (23): 12297-302; Barendt P A, Ng D T, McQuade C N, Sarkar C A (2013). “Streamlined Protocol for mRNA Display”. ACS Combinatorial Science. 15 (2): 77-81; Fukuda I, Kojoh K, Tabata N, et al. (2006). “In vitro evolution of single-chain antibodies using mRNA display”. Nucleic Acids Research. 34 (19): e127, which are each incorporated by reference herein as if expressed in their entireties and can be adapted for use with the present invention in view of this disclosure. In certain example embodiments, the engineered display system is based on an mRNA display system, including, but not limited to the exemplary mRNA display systems previously described. In some embodiments, the display molecule is a puromycin.

Various DNA display systems have been described. For example, an encoding DNA can be directly fused or operatively coupled to an affinity molecule. In this context, the DNA can be analogous to a “display molecule” as the term is used in connection with the present invention. Other DNA display systems include CIS display and CDT display systems as are further described elsewhere herein.

In some embodiments, the engineered display system is based on a CIS display system. Various CIS display systems have been described. See e.g., Odergrip et al. PNAS 2004 1010(9):2806-2810, which is incorporated by reference herein as if expressed in its entirety and can be adapted for use with the present invention in view of this disclosure. In some embodiments the engineered display system is based on a CIS display system. In some embodiments, the display molecule is RepA polypeptide. RepA, via its cis activity, can bind to DNA and thus couple an affinity molecule of the present invention to an encoding polynucleotide and/or sequencing molecule of the present invention.

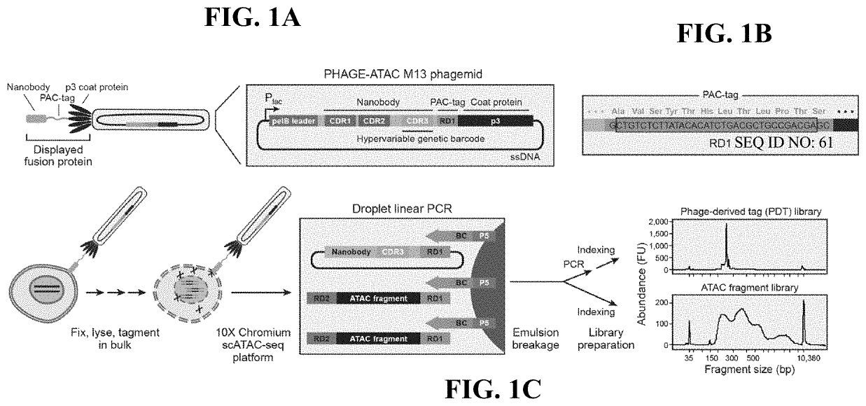

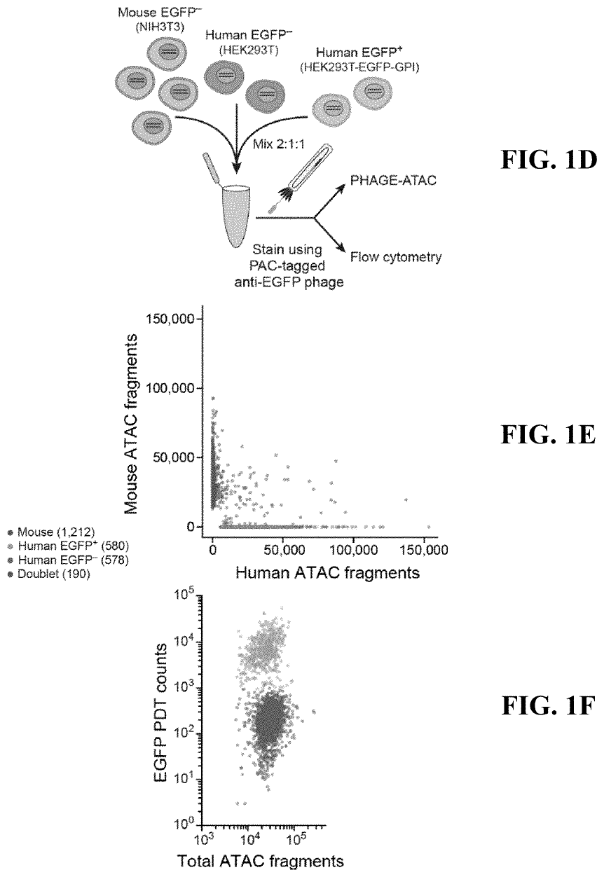

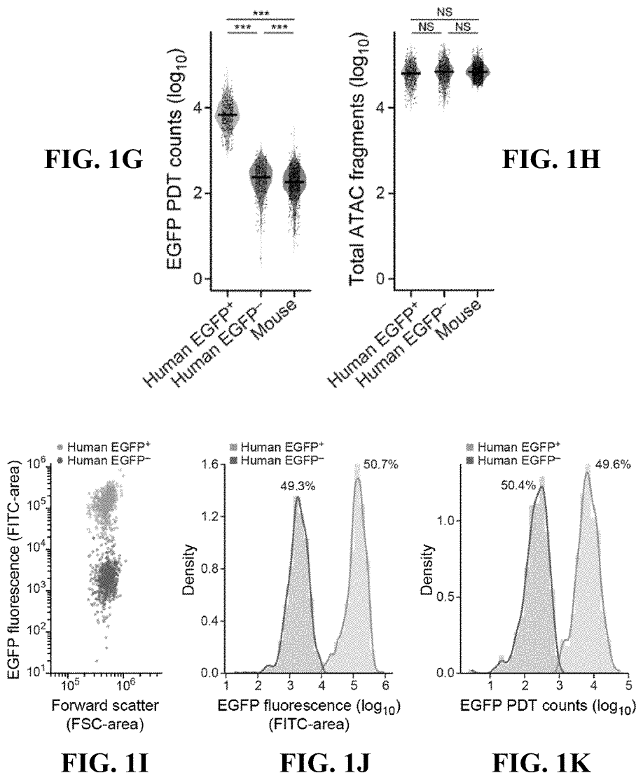

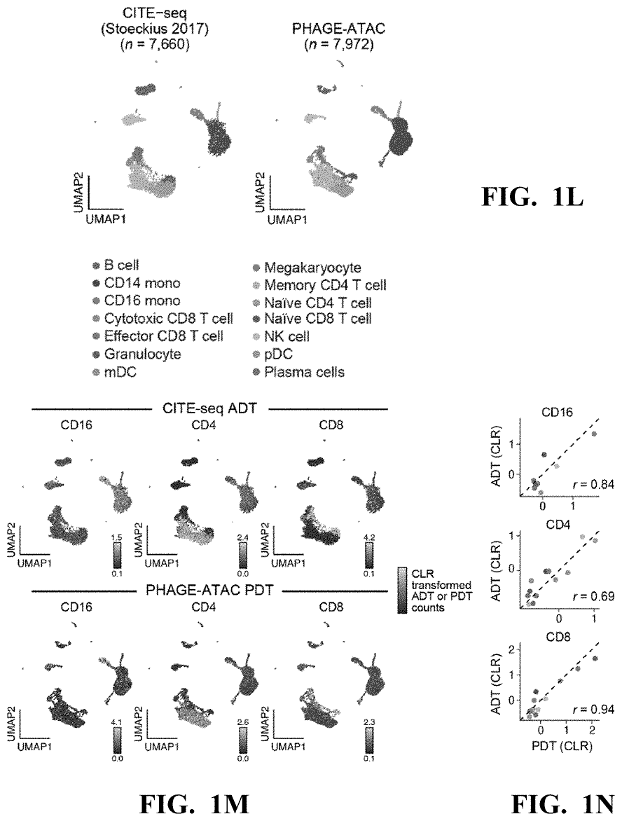

In some embodiments, the engineered display system is based on a covalent DNA display system. Various covalent DNA display systems have been described. See e.g., Reiersen et al. 2005. 33(1): e10, particularly at FIG. 1; FitzGerald. 2000. Res. Focus. 5(6):253-258; and Sergeeva et al. 2006. Adv. Drug Deliv. Rev. 58:1622-1654, which are each incorporated by reference herein as if expressed in their entireties and can be adapted for use with the present invention in view of this disclosure. Generally, these systems exploit the endonuclease P2A. In some embodiments, the engineered display system is based on a covalent DNA display system. In some embodiments, the display molecule is P2A endonuclease.

In some embodiments, the engineered display system is based on a eukaryotic system in which the display molecule is a surface expressed protein. Any suitable eukaryotic cell can be used. In some embodiments, the cell is a yeast cell. In some embodiments, the cell is an insect cell. In some embodiments, the cell is a mammalian cell. In some embodiments, the cell is a human cell. In some embodiments, the cell is an immune cell. In some embodiments, the cell is an antigen presenting cell. In some embodiments, the cell is a T cell, a macrophage, or a B cell.

In some embodiments, the engineered display system is a viral-based system where the affinity molecule is coupled to a capsid protein and displayed on the capsid surface. Suitable non-bacterial viral systems include bacteriophages or non-bacterial virial systems. Non-bacterial viral systems include, but are not limited to, lentiviral/retroviral, adenoviral, adeno-associated viral systems, or any other virus. Such viruses are generally known and are included within the scope of the present disclosure.

In certain example embodiments, the sequencing molecule is a barcode polynucleotide, an index polynucleotide, a primer-binding site, an adapter polynucleotide, or any combination thereof. In certain example embodiments, the engineered display construct is a viral vector, a non-viral vector, or a naked polynucleotide, or a system thereof.

In certain example embodiments, the engineered display construct is an expression vector.

In certain example embodiments, the engineered display construct is a prokaryotic cell expression vector or a eukaryotic cell expression vector.

In certain example embodiments, the engineered display construct is a phagemid.

In certain example embodiments, the genetically encoded display molecule is a genetically encoded capsid polypeptide, a genetically encoded prokaryotic cell surface polypeptide, a genetically encoded eukaryotic cell surface polypeptide, a genetically encoded P2A endonuclease polypeptide, a genetically encoded RepA polypeptide, a genetically encoded ribosome protein, or a genetically encoded ribosomal RNA.

As previously described, in some embodiments, the display construct comprises a genetically encoded display molecule linker. As used herein, a “display molecule linker” refers to a linking molecule that facilitates fusing, covalent bonding, operatively coupling, or otherwise associating a display molecule with another molecule of a engineered display system and/or engineered display construct herein. Thus, a “genetically encoded display molecule linker” is a polynucleotide that encodes or is a display molecule linker. In some embodiments, such as in the context of a ribosome display-based engineered display system, the spacer lacking a stop codon is a genetically encoded display molecule linker. In some embodiments, such as in the context of an mRNA display-based engineered display system, a segment of polynucleotide that serves as a binding site for a puromycin molecule is a genetically encoded display molecule linker. Other linkers and display molecule pairs will be appreciated in view of the description provided herein.

Embodiments disclosed herein provide engineered phagemids including a genetically encoded capsid polypeptide; a genetically encoded affinity molecule; and a genetically encoded sequencing molecule, wherein the genetically encoded sequencing molecule is fused to or is operatively coupled to the genetically encoded affinity molecule and the genetically encoded capsid polypeptide. In some embodiments, the genetically encoded sequencing molecule is molecule is fused or is operatively coupled in frame to the genetically encoded affinity molecule, the genetically encoded capsid polypeptide, or both. In some embodiments the genetically encoded sequencing molecule is fused to or is operatively coupled to the 5′ end or elsewhere upstream of the genetically encoded affinity molecule or the genetically encoded capsid polypeptide. In some embodiments the genetically encoded sequencing molecule is fused to or is operatively coupled to the 3′ end or elsewhere downstream of the genetically encoded affinity molecule the genetically encoded capsid polypeptide. In some embodiments, the genetically encoded sequencing molecule is not an encoding polynucleotide of the genetically encoded affinity molecule. In other words, in some embodiments the genetically encoded sequencing molecule does not encode one or more regions of the affinity molecule that is incorporated into an engineered bacteriophage described herein. However, even in some of these embodiments and others, the genetically encoded sequencing molecule can be operatively coupled to the genetically encoded engineered capsid and/or affinity molecule and translated such that a polypeptide tag that can be fused to or otherwise operatively coupled to an expressed affinity molecule and/or engineered capsid is produced (see for example, PAC-tag in FIGS. 1A-1C). In some embodiments, the translated sequencing molecule (now a polypeptide tag) is optionally detected using a suitable protein detection technique and the genetically encoded sequencing molecule sequenced from the engineered phagemid contained within the same engineered bacteriophage (see e.g., FIGS. 1A-1C). The Working Examples elsewhere herein demonstrate a non-limiting exemplary engineered phagemid of the present disclosure.

### Genetically Encoded Display Molecule

In some embodiments, the engineered display construct includes a genetically encoded display molecule. In other words, in some embodiments, the engineered display construct includes a polynucleotide that encodes a display molecule. As used herein, “display molecule” refers to a molecule, such as a polypeptide or a small molecule, that is operatively coupled to the affinity molecule so as to “display” the affinity molecule and/or serve as an anchor and/or tether for the affinity molecule and/or sequencing molecule. In certain example embodiments, the genetically encoded display molecule is a genetically encoded capsid polypeptide, a genetically encoded prokaryotic cell surface polypeptide, a genetically encoded eukaryotic cell surface polypeptide, a genetically encoded P2A endonuclease polypeptide, or a genetically encoded RepA polypeptide. Display molecules are further described elsewhere herein.

In some embodiments, the engineered display construct is an engineered phagemid. In some embodiments, the engineered phagemid includes a genetically encoded capsid polypeptide. In other words, in some embodiments, the engineered phagemid includes a polynucleotide that encodes a capsid polypeptide. Capsid polypeptides are discussed elsewhere herein, such as with respect to the engineered bacteriophages. In some embodiments, the engineered phagemid includes a genetically encoded major capsid polypeptide. In some embodiments, the engineered phagemid includes a genetically encoded minor capsid polypeptide.

In some embodiments, the engineered phagemid includes one or more, such as 1, 2, 3, 4, 5, 6, 7, 8, 9, 10 or more, genetically encoded capsid polypeptides. In some embodiments, the genetically encoded sequencing molecule is fused to or is operatively coupled to one or more of the two or more genetically encoded capsid polypeptides. In some embodiments, the genetically encoded capsid polypeptides are homogenous. In some embodiments, the genetically encoded capsid polypeptides are heterogenous.

The genetically encoded capsid polypeptide can be any suitable genetically encoded bacteriophage capsid polypeptide. In some embodiments, the genetically encoded bacteriophage capsid polypeptide(s) is/are or includes a genetically encoded lysogenic bacteriophage capsid polypeptide. In some embodiments, the genetically encoded bacteriophage capsid polypeptide(s) is/are or includes a genetically encoded lytic bacteriophage capsid polypeptide. In some embodiments, the genetically encoded bacteriophage capsid polypeptide(s) is/are or includes a genetically encoded Caudovirale and/or Ligamenvirales bacteriophage capsid polypeptide. In some embodiments, the genetically encoded bacteriophage capsid polypeptide(s) is/are or includes a genetically encoded Ackermannviridae, Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttaviridae, Inoviridae, Leviviridae, Microviridae, Plasmaviridae, Pleolipoviridae, Portogloboviridae, Sphaerolipoviridae, Spiraviridae, Tectiviridae, Tristromaviridae, Turriviridae, and combinations thereof.

In some embodiments, the genetically encoded capsid polypeptide is a M13 phage genetically encoded capsid polypeptide. In some embodiments, the M13 phage genetically encoded capsid polypeptide is a P3, P6, P7, P8, or P9 genetically encoded capsid polypeptide.

### Genetically Encoded Affinity Molecule

In some embodiments, the engineered display construct (e.g., a phagemid) includes a genetically encoded affinity molecule. In other words, in some embodiments, the engineered display construct (e.g., a phagemid) includes a polynucleotide that encodes an affinity molecule. In some embodiments, the genetically encoded affinity molecule encodes a polynucleotide, peptide, polypeptide, or a combination thereof. Affinity molecules are also discussed and described in greater detail elsewhere herein, such as with respect to the engineered display systems, (e.g. engineered bacteriophages and others) and below.

As used herein, “affinity molecule” refers to any molecule, chemical, biological, or otherwise, that specifically and/or preferentially binds, associates with, and/or otherwise functionally interacts with another molecule or group of a specific type of molecules (also referred to as a target molecule or target molecules) over other molecules such that a difference between the interactions/binding/association between a target molecule and a non-target molecule can be observed and no detectable cross reactivity is observed unless within a specific desired grouping of similar or related target molecules. As used herein, the term “specific binding”, “specifically bind”, and the like refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶M or less, 10⁻⁷ M or less, 10⁻⁸ M or less, 10⁻⁹M or less, 10⁻¹⁰ M or less, 10⁻¹¹M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

Suitable affinity molecules include, without limitation, polynucleotides (e.g. DNA and RNA), peptides and polypeptides, (e.g. antibodies and fragments thereof, and fragments thereof, ligands, receptors, etc.), chemical compounds (e.g. ligands), and engineered scaffolds (e.g. engineered binding scaffolds, engineered antibodies, aptamers, affibodies, nanobodies, avimers, engineered nanobodies, engineered protein scaffolds) (see also e.g. A. Skerra. J Molec. Recognition. 2000. 13(4) https://doi.org/10.1002/1099-1352(200007/08)13:4<167::AID-JMR502>3.0.CO;2-9; Konning and Kolmar. 2018. Microbial Cell Factories 17(32); Gebaure and Skerra. 2009. Curr. Op. Chem. Biol. 13(3):245-255; Simeon and Chen. Protein. Cell. 2018. 9(1):3-14; Gebauer and Skerra. 2020. Ann. Rev. Pharmacol. Toxicol. 60:391-415.

The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). “Antibody” includes monovalent and multivalent antibodies. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

As used herein, “nanobody” refers to a single-domain antibody fragment that is capable of specifically binding an antigen. Nanobodies can be engineered to have desired antigen binding capabilities. Nanobodies can be based on heavy chain or light chain domains. See e.g. Arbabi Ghahroudi M, Desmyter A, Wyns L, Hamers R, Muyldermans S (September 1997). “Selection and identification of single domain antibody fragments from camel heavy-chain antibodies”. FEBS Letters. 414 (3): 521-6. doi:10.1016/S0014-5793(97)01062-4; Ward E S, Güssow D, Griffiths A D, Jones P T, Winter G (October 1989). “Binding activities of a repertoire of single immunoglobulin variable domains secreted from Escherichia coli”. Nature. 341 (6242): 544-6. Bibcode:1989Natur.341..544W. doi:10.1038/341544a0; Holt L J, Herring C, Jespers L S, Woolven B P, Tomlinson I M (November 2003). “Domain antibodies: proteins for therapy”. Trends in Biotechnology. 21 (11): 484-90. doi:10.1016/j.tibtech.2003.08.007; Borrebaeck C A, Ohlin M (December 2002). “Antibody evolution beyond Nature”. Nature Biotechnology. 20 (12): 1189-90. doi:10.1038/nbt1202-1189; Van de Broek B, Devoogdt N, D'Hollander A, Gijs H L, Jans K, Lagae L, et al. (June 2011). “Specific cell targeting with nanobody conjugated branched gold nanoparticles for photothermal therapy”. ACS Nano. 5 (6): 4319-28. doi:10.1021/nn1023363.

As used herein, the term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g., the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG—IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains). In some embodiments, the VH domain is a human VH domain.

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

As used herein, “affibody” refers to small (typically around 6.5 kDa) non-immunoglobulin engineered proteins based on a three-helix bundle domain framework that is based on a 58-amino-acid Z-domain scaffold, derived from one of the IgG-binding domains of staphylococcal protein A and can be engineered for desired target recognition. See e.g., Frejd and Kim. 2017. Exp. Mol. Med. 49(3):e306; Löfblom J, et al. FEBS Lett. 2010 Jun. 18; 584(12):2670-80. doi: 10.1016/j.febslet.2010.04.014. Epub 2010 Apr. 11; and Nygren, P. A. FEBS J. 2008 June; 275(11):2668-76.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

In certain embodiments, the affinity molecule is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-0-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to, those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colo.). In certain embodiments, the present invention may utilize any aptamer containing any modification as described herein.

In some embodiments, the affinity molecule is a chemical small molecule, such as a small molecule receptor ligand. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

The genetically encoded affinity molecule can be included in the phagemid such that when expressed, the genetically encoded affinity molecule can be fused to or operably coupled to a capsid protein, the genetically encoded sequencing molecule or both. In some embodiments, the genetically encoded affinity molecule can be included in the phagemid such that when expressed, the affinity molecule is expressed on the surface of an assembled phage capsid. In this way, the affinity molecule can result in specific binding, association, or other interaction with a target on the surface or in a cell or nucleus.

In some embodiments, the phagemid can include two or more genetically encoded affinity molecules, such as 2, 3, 4, 5, 6, 7, 8, 9, 10 or more. In some embodiments, the genetically encoded sequencing molecule is fused to or is operatively coupled to one or more of the two or more genetically encoded affinity molecules. In some embodiments, a genetically encoded capsid polypeptide is fused to or is operatively coupled to one or more of the two or more genetically encoded affinity molecules. In some embodiments, the two or more genetically encoded affinity molecules are homogenous. In some embodiments, the two or more genetically encoded affinity molecules are heterogenous.

In some embodiments, the genetically encoded affinity molecule is capable of generating an affinity molecule polypeptide capable of specifically binding a predetermined target present on the surface of and/or inside of a cell and/or nucleus. In some embodiments, the predetermined target is a microorganism protein; a cancer-associated protein; an immune checkpoint protein or checkpoint inhibitor; a cell-type marker; a cell-state marker; a non-cancer disease or condition biomarker; or a combination thereof.

Microorganism proteins include any surface or intracellular or intranuclear proteins present in a microorganism. As used herein, “microorganism” refers to microscopic organisms and includes, but are not limited to, bacteria, viruses, fungi, algae, yeasts, protozoa, worms, spirochetes, single-celled and multi-celled organisms that are included in classification schema as prokaryotes, eukaryotes, Archea, bacteria and those that are known to those skilled in the art. In certain example embodiments, the infectious agent is pathogenic. In certain example embodiments, the infectious agent is non-pathogenic.

As used herein “cancer” refers to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytoma, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sézary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “immune checkpoint” refers to normal parts of the immune system that function to prevent an immune response from being so great that it damages or destroys healthy cells. Immune checkpoints can engage when e.g., proteins on the surface immune cells (e.g. T cells) recognize and bind to partner proteins (called immune checkpoint proteins) on other cells. Such binding results in a signal that shuts down or turns off the immune cell(s) (e.g., T cells) to prevent aberrant destruction of healthy cells. In some diseases, such as cancer, diseased/cancerous cells will express the immune checkpoint protein such that when an immune cell binds the checkpoint protein on the diseased/cancerous cell, the immune system is prevented from destroying the cell. In this way diseases, such as cancer, can hijack the immune checkpoint system to evade destruction by the immune system.

As used herein, “immune checkpoint protein” refers to proteins or other molecules that are on the surface of certain immune cells (e.g., T cells) or their binding partner on the surface of another cell whose pairing forms an immune checkpoint and whose binding results in signal generation that lessens or shuts down a damaging or lethal immune response towards cell bound by the certain immune cell. Exemplary checkpoint proteins include, but are not limited to, PD1, CD28, CTLA-4, ICOS, TMIGD2, 4-1BB, CD160, LIGHT, LAG3, CD27, OX40, C40L, GITR, DNAM-1, TIGT, CD96, TIM3, Adenosine A2a receptor, CEACAM1, SIRP alpha, CD200R, DR3, PD-L1, PD-L2, CD80, CD86, ICOS ligand, B7-H3, B7-H4, VISTA, B7-H7, HVEM, MHC Class I, MHC Class II, OX40L, CD70, CD40, GITRL, CD155, CD48, Calectin-9, Adenosine, IDO, TDO, CECAM1, CD47, BTN2A1, CD200, and TLA1.

As used herein, “immune checkpoint inhibitor” refers to compounds and agents that can block immune checkpoint proteins from binding with their binding partner(s), which can prevent an “off” signal from being sent and allowing activation of certain immune cells and functions. Exemplary immune checkpoint inhibitors include, but are not limited to, antibodies, engineered scaffolds, and the like that bind a checkpoint protein, and small molecule immune checkpoint inhibitors. Exemplary PD1 immune checkpoint inhibitors include, but are not limited to, Nivolumab, PembrolizumabIn, Pidilizumab, AMP-224. Exemplary PD-L1 immune checkpoint inhibitors include, but are not limited to, BMS-936559, MEDI4736, MPDL3280A, Avelumab. Exemplary CTLA-4 immune checkpoint inhibitors include, but are not limited to, Tremelimumab. Exemplary B7-H3 immune checkpoint inhibitors include, but are not limited to, MGA271. Exemplary IDO immune checkpoint inhibitors include, but are not limited to, Indoximod, INCB024360. Exemplary KIR immune checkpoint inhibitors include, but are not limited to, Lirilumab. Exemplary B7-H3 immune checkpoint inhibitors include, but are not limited to, BMS-986016. See also e.g. Howard (Jack) West, M D et al. Immune Checkpoint Inhibitors. JAMA Oncol. 2015; 1(1):115. Julie R. Brahmer et al. Immune Checkpoint Inhibitors: Making Immunotherapy a Reality for the Treatment of Lung Cancer. Cancer Immunol Res August 2013 1; 85; Darvin et al. Exp Mol Med. 2018 Dec. 13; 50(12):1-11. doi: 10.1038/s12276-018-0191-1; E. Hui. Cell Biol. 2019 Mar. 4; 218(3):740-741. doi: 10.1083/jcb.201810035.

As used herein, “cell type” refers to the more permanent aspects (e.g., a hepatocyte typically can't on its own turn into a neuron) of a cell's identity. Cell state can be thought of as the permanent characteristic profile or phenotype of a cell. Cell types are often organized in a hierarchical taxonomy, types may be further divided into finer subtypes; such taxonomies are often related to a cell fate map, which reflect key steps in differentiation or other points along a development process. Wagner et al., 2016. Nat Biotechnol. 34(111): 1145-1160. As used herein, a “cell type marker” refers to one or more proteins, peptides, polynucleotides, or other molecule whose expression signature is unique to one specific cell type as compared to a different cell type.

As used herein, “cell state” are used to describe transient elements of a cell's identity. Cell state can be thought of as the transient characteristic profile or phenotype of a cell. Cell states arise transiently during time-dependent processes, either in a temporal progression that is unidirectional (e.g., during differentiation, or following an environmental stimulus) or in a state vacillation that is not necessarily unidirectional and in which the cell may return to the origin state. Vacillating processes can be oscillatory (e.g., cell-cycle or circadian rhythm) or can transition between states with no predefined order (e.g., due to stochastic, or environmentally controlled, molecular events). These time-dependent processes may occur transiently within a stable cell type (as in a transient environmental response), or may lead to a new, distinct type (as in differentiation). Wagner et al., 2016. Nat Biotechnol. 34(111): 1145-1160. As used herein, a “cell state marker” refers to one or more proteins, peptides, polynucleotides, or other molecule whose expression signature is unique to one specific cell state as compared to a different cell state.

Exemplary non-cancerous diseases include, but are not limited to, autoimmune diseases, allergies and asthma, intestinal diseases and disorders, heart disease and disorders, lung diseases and disorders, sinus diseases and disorders, kidney diseases and disorders, infectious diseases, liver diseases, central and peripheral nervous system diseases and disorders, inflammatory diseases and disorders, pancreatic diseases and disorders, brain diseases and disorders, muscle diseases and disorders, bone diseases and disorders, connective tissue diseases and disorders, metabolic diseases and disorders, skin diseases and disorders, eye diseases and disorders, ear diseases and disorders, nose diseases and disorders, dental diseases and disorders, stomach diseases and disorders, bladder diseases and disorders, prostate diseases and disorders, urinary system diseases and disorders, vaginal, ovarian, and uterine diseases and disorders, testis diseases and disorders, breast diseases and disorders, esophagus diseases and disorders, vascular diseases and disorders, blood disease and disorders, pulmonary diseases and disorders, cerebrovascular diseases and disorders, cardiovascular diseases and disorders, and infections caused by a microorganism.

### Genetically Encoded Sequencing Molecule

In some embodiments, the engineered display construct includes a genetically encoded sequencing molecule, wherein the genetically encoded sequencing molecule is fused to or is operatively coupled to the genetically encoded affinity molecule and the genetically encoded display molecule. In other words, in some embodiments, the engineered display construct includes a polynucleotide that is or encodes a sequencing molecule. As used herein, “sequencing molecule” refers to a polynucleotide that has a specific function or role in sequencing such as a barcode, unique molecular identifier, adaptor, primer binding site, and the like. In some embodiments, the sequencing molecule is an engineered display construct specific, engineered display system specific, an engineered phagemid specific, bacteriophage specific, affinity molecule specific, cell specific, nucleus specific, or a combination thereof. In some embodiments, the genetically encoded sequencing molecule is or contains an adaptor that is or is compatible with a sequencing method such as a 10× genomics sequencing adaptor, Illumina sequencing adaptor, an in-situ sequencing adaptor (e.g., an optical read out adaptor), and the like.

### Nucleic Acid Barcode, Barcode, and Unique Molecular Identifier (UMI)

The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, single nucleus, engineered phagemid, engineered bacteriophage, affinity molecule, viral vector, labeling ligand (e.g., an aptamer), protein, shRNA, sgRNA or cDNA such that multiple species can be sequenced together. A nucleic-acid based barcode is a short sequence of nucleotides (for example, DNA, RNA, or combinations thereof) that is used as an identifier for an associated molecule, such as a target molecule and/or target nucleic acid. A nucleic acid barcode can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Typically, a nucleic acid barcode is used to identify a target molecule and/or target nucleic acids as being from a particular compartment (for example a discrete volume), having a particular physical property (for example, affinity, length, sequence, etc.), or having been subject to certain treatment conditions. Target molecule and/or target nucleic acid can be associated with multiple nucleic acid barcodes to provide information about all of these features (and more). Methods of generating nucleic acid-barcodes are disclosed, for example, in International Patent Application Publication No. WO/2014/047561.

Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell.

In preferred embodiments, sequencing is performed using unique molecular identifiers (UMI). The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR or multiple displacement amplification (MDA).

In certain embodiments, an UMI with a random sequence of between 4 and 20 base pairs is added to a template, which is amplified and sequenced. In preferred embodiments, the UMI is added to the 5′ end of the template. Sequencing allows for high resolution reads, enabling accurate detection of true variants. As used herein, a “true variant” will be present in every amplified product originating from the original clone as identified by aligning all products with a UMI. Each clone amplified will have a different random UMI that will indicate that the amplified product originated from that clone. Background caused by the fidelity of the amplification process can be eliminated because true variants will be present in all amplified products and background representing random error will only be present in single amplification products (See e.g., Islam S. et al., 2014. Nature Methods No:11, 163-166). Not being bound by a theory, the UMI's are designed such that assignment to the original can take place despite up to 4-7 errors during amplification or sequencing. Not being bound by a theory, an UMI may be used to discriminate between true barcode sequences.

Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached or operatively coupled to, or “tagged,” to a genetically encoded affinity molecule and/or capsid and thus an expressed affinity molecule and/or capsid as described elsewhere herein. Binding of an affinity molecule to a target can result in indirect attachment of the barcode, such as the genetically encoded sequencing molecule described herein, to the target.

One or more additional barcodes can be optionally included to the phagemid, bacteriophage or target. One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associate with (for example, attached to) a target molecule, as described herein.

Affinity molecules and/or target molecules can be optionally labeled with multiple barcodes in combinatorial fashion (for example, using multiple barcodes bound to one or more specific binding agents (also referred to herein as affinity molecules) that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SB S3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type NNNNNNNNNNNN (SEQ ID NO: 1).

A nucleic acid identifier can further include a unique molecular identifier and/or additional barcodes specific to, for example, a common support to which one or more of the nucleic acid identifiers are attached. Thus, a pool of target molecules or affinity molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled affinity molecules, and/or target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites. A nucleic acid barcode (or a concatemer thereof), a genetically encoded affinity molecule, an affinity molecule, a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others, Drop-Seq, single cell sequencing, single nucleus sequencing, ATAC-seq, and combinations and variations thereof. In some embodiments, the sequence of labeled target molecules is determined by non-sequencing based methods. For example, variable length probes or primers can be used to distinguish barcodes (for example, origin-specific barcodes) labeling distinct target molecules and/or affinity molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid) or a type of target for a particular affinity molecule. For example, in a pool of labeled target or affinity molecules containing multiple types of target molecules or affinity molecules, polypeptide target molecules or affinity molecules can receive one identifying sequence, while target nucleic acid molecules or affinity molecules can receive a different identifying sequence. Such identifying sequences can be used to selectively amplify barcodes labeling particular types of target molecules and/or affinity molecules, for example, by using PCR primers specific to identifying sequences specific to particular types of target molecules and/or affinity molecules. For example, barcodes labeling polypeptide target molecules or affinity molecules can be selectively amplified from a pool, thereby retrieving only the barcodes from the polypeptide subset of the target molecule pool.

A nucleic acid barcode can be sequenced, for example, after cleavage, to determine the presence, quantity, or other feature of the target molecule via the affinity molecule proxy. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

### Optically Detectable Barcodes

Optically detectable barcodes are barcodes that can be detected with light or fluorescence microscopy. In certain example embodiments, the optical barcodes may comprise a sub-set of fluorophores or quantum dots of distinguishable colors from a set of defined colors. In certain example embodiments, beads are labeled with different ratios of dyes to form the set of defined colors from which the optical barcodes may be derived. For example, the beads may be polystyrene beads labeled with biotin conjugated dyes. Alternatively, the optical barcodes may be derived using a combination of optically detectable objects. For example, an optical barcode may be defined from a set of objects that can vary in size, shape, color, or any combination thereof that is distinguishable by light or fluorescence microscopy.

### Barcodes Coupled to Solid Substrate

In some embodiments, the origin-specific barcodes or a barcode capable of specifically binding to an origin specific barcode are reversibly or irreversibly coupled to a solid or semisolid substrate. In some embodiments, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules. In some embodiments the substrate is a bead, such as a hydrogel bead. In some embodiments the substrate is a 10× genomics sequencing bead.

Barcode with Cleavage Sites

A nucleic acid barcode may be cleavable from a specific binding agent, for example, after the specific binding agent has bound to a target molecule. In some embodiments, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, at least one cleavage site is oriented such that the cleavage at the site releases the origin-specific barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage.

### Barcode Adapters

In some embodiments, the affinity molecule and/or genetically encoded affinity molecule is attached or coupled to a barcode receiving adapter, which is optionally origin specific, such as a nucleic acid. In some examples, the optionally origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached or otherwise coupled to) a sequencing substrate, such as a bead.

In some embodiments, the barcode receiving adaptor may be associated with (for example, attached) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

Barcode with Capture Moiety

In some embodiments, an origin-specific barcode further includes a capture moiety, covalently or non-covalently linked. Thus, in some embodiments the origin-specific barcode, and anything bound or attached thereto, that include a capture moiety are captured with a specific binding agent that specifically binds the capture moiety. In some embodiments, the capture moiety is adsorbed or otherwise captured on a surface. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include: incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments, the specific binding agent has been immobilized for example on a solid support, thereby isolating the origin-specific barcode.

Barcode with Detectable Tags

The barcodes herein may comprise one or more detectable tags. In some examples, a detectable tag may comprise a detectable oligonucleotide tag is an oligonucleotide that can be detected by sequencing of its nucleotide sequence and/or by detecting non-nucleic acid detectable moieties it may be attached to.

The oligonucleotide tags may be randomly selected from a diverse plurality of oligonucleotide tags. In some instances, an oligonucleotide tag may be present once in a plurality or it may be present multiple times in a plurality. In the latter instance, the plurality of tags may be comprised of a number of subsets each comprising a plurality of identical tags. In some important embodiments, these subsets are physically separate from each other. Physical separation may be achieved by providing the subsets in separate wells of a multiwell plate or separate droplets from an emulsion. It is the random selection and thus combination of oligonucleotide tags that results in a unique label. Accordingly, the number of distinct (i.e., different) oligonucleotide tags required to uniquely label a plurality of agents can be far less than the number of agents being labeled. This is particularly advantageous when the number of agents is large (e.g., when the agents are members of a library).

The oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the non-nucleic acid detectable moiety.

In some embodiments, a detectable oligonucleotide tag comprises one or more non-oligonucleotide detectable moieties. Examples of detectable moieties include fluorophores, microparticles including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, haptens, and other detectable moieties known to those skilled in the art. In some embodiments, the detectable moieties are quantum dots. Methods for detecting such moieties are described herein and/or are known in the art.

Thus, detectable oligonucleotide tags may be, but are not limited to, oligonucleotides comprising unique nucleotide sequences, oligonucleotides comprising detectable moieties, and oligonucleotides comprising both unique nucleotide sequences and detectable moieties.

In some cases, the detectable tag comprises a labeling substance, which is detectable by spectroscopic, photochemical, biochemical, immunochemical, electrical, optical or chemical means. Such tags include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Detectable tags may be detected by many methods. For example, radiolabels may be detected using photographic film or scintillation counters, and fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 125I, 3H, and 131I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added. Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-di ethyl amino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. A fluorescent label may be a fluorescent protein, such as blue fluorescent protein, cyan fluorescent protein, green fluorescent protein, red fluorescent protein, yellow fluorescent protein or any photoconvertible protein. Colorimetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code. Advantageously, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation. In some embodiments, the detectable moieties may be quantum dots.

### Split-Pool Barcoding

In some embodiments, the nucleic acids molecules, e.g., the fragmented genomic DNA and the cDNA, may be barcoded by a split-pool method. In some embodiments, the split-pool method may be performed on a sample comprising nuclei containing the fragmented genomic DNA and the cDNA herein. In such cases, the fragmented genomic DNA and the cDNA remain in nuclei after generation. The nuclei may remain intact during the split-pool process. In certain examples, the nuclei are isolated from cells. For example, the cells may be lysed and the nuclei are released, but remain intact and contain the fragmented genomic DNA and the cDNA. In certain examples, the nuclei remain in the cells, which are made permeable so the nucleic acids in the cells (e.g., in the nuclei) can access reaction reagents and the fragmented DNA and the cDNA can be generated inside cells.

In general, the split-pool method may comprise splitting a sample comprising nuclei into discrete volumes in partitions, each partition containing a unique first barcode; ligating the first barcode to nucleic acids in each partition; and pooling the discrete partitions to a first pooled sample. The process may be performed once. The process may be repeated. For example, the split-pool method may further comprise splitting the first pooled sample into discrete partitions, each partition containing a unique second barcode; ligating the second barcode to nucleic acids in each partition; and pooling the discrete partitions to make a second pooled sample. The splitting and pooling steps may be repeated for at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, or at least 500 times. In some cases, the splitting and pooling steps may be repeated once, twice, three times, or four times. In some cases, the pooled sample may be used for further processing and analysis. In certain cases, the split samples in partitions may be used for further processing and analysis. In some cases, the split-pooling (one or multiple rounds) may be performed for barcode ligation. Multiple rounds of split-pooling may create barcode possibilities to identify cells, thus increase the throughput of analysis methods.

After split-pool steps, each nucleic acid molecule may comprise one or a combination of barcodes. In a split-pool step, nucleic acid molecules in a nucleus or cell are split together, nucleic acid molecules from or derived from the same cell may receive the same barcode or barcode combination. Such barcode or barcode combination may comprise a unique barcode sequence, which may be used as an identifier of cell origin of the nucleic acid molecules. In some embodiments, the split-pool-ligation approach may be modified to a split-pool-hybridization-ligation approach. For example, the barcodes may be hybridized to nuclei during each round without adding ligase. After several rounds of hybridization, the nuclei may be washed and then resuspended in ligation mixture. This approach may provide similar or better yield than split-pool-ligation approach. The overall cost for ligase may be much lower.

In some embodiments, nucleic acids in the split-pool process may comprise ligation handles. The ligation handle may comprise a restriction site for producing an overhang complementary with a first index sequence overhang, and wherein the method further comprises digestion with a restriction enzyme. The ligation handle may comprise a nucleotide sequence complementary with a ligation primer sequence and wherein the overhang complementary with a first index sequence overhang is produced by hybridization of the ligation primer to the ligation handle. The ligation handles may be generated before the split-pool process. For example, the ligation handles may be generated during the fragmentation, tagmentation, and/or RT-PCR process. Alternatively or additionally, the ligation handles may be generated during the split-pool process.

### Other Barcoding Embodiments

DNA barcoding is also a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102(23):8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

It has been suggested that a desirable locus for DNA barcoding should be standardized so that large databases of sequences for that locus can be developed. Most of the taxa of interest have loci that are sequencable without species-specific PCR primers. CBOL Plant Working Group, “A DNA barcode for land plants” PNAS 106(31):12794-12797 (2009). Further, these putative barcode loci are believed short enough to be easily sequenced with current technology. Kress et al., “DNA barcodes: Genes, genomics, and bioinformatics” PNAS 105(8):2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105(8):2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31):12569 (2009).

Software for DNA barcoding requires integration of a field information management system (HMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94). Such barcoding approaches can be used in context with the present disclosure and embodiments herein.

### Engineered Display Systems

Described in certain example embodiments herein are engineered display systems comprising the engineered display construct described elsewhere herein.

In certain example embodiments, the display system is an engineered viral display system, an engineered prokaryotic cell display system, an engineered eukaryotic cell display system, an engineered mRNA display system, engineered ribosome display system, or an engineered DNA display system.

In certain example embodiments, the engineered display system is an engineered bacteriophage; an engineered non-bacteria virus; an engineered bacterial cell; an engineered yeast cell; an engineered mammalian cell; an engineered insect cell; an engineered DNA display system; an engineered covalent display system; or an engineered CIS display system, an engineered mRNA display system, or an engineered ribosome display system.

In certain example embodiments, the engineered display system further comprises: a display molecule; an affinity molecule; and a sequencing polypeptide, wherein the sequencing molecule polypeptide is fused to or operatively coupled to the display molecule and/or the affinity polypeptide.

In certain example embodiments, the display molecule comprises a capsid polypeptide, a yeast cell surface polypeptide, a bacteria cell surface polypeptide, a mammalian cell surface polypeptide, an insect cell surface polypeptide, a puromycin, a ribosome or a component thereof, a P2A endonuclease polypeptide, or a RepA polypeptide, or other small molecule.

In certain example embodiments, the affinity molecule comprises a peptide, polypeptide, polynucleotide, a small molecule, or a combination thereof.

In certain example embodiments, the affinity molecule is an antibody or fragment thereof.

In certain example embodiments, wherein the affinity molecule comprises or consists of a human or humanized antibody VH domain. In some embodiments, the affinity molecule comprises or consist of a VH domain.

In certain example embodiments, the display system is a bacteriophage.

In certain example embodiments, the display molecule is a capsid polypeptide.

In certain example embodiments, the display molecule is a major capsid polypeptide or a minor capsid polypeptide.

In some embodiments, the engineered display system is an engineered bacteriophage. Bacteriophages are viruses that infect bacteria. See e.g., Clokie et al. 2011. Bacteriophage. January-February; 1(1):31-24. The engineered bacteriophages described herein can contain one or more engineered phagemids described in greater detail elsewhere herein. In some embodiments, the engineered bacteriophages described herein can include an engineered capsid comprising: a capsid polypeptide; an affinity molecule; and a sequencing molecule polypeptide, wherein the sequencing molecule polypeptide is fused to or operatively coupled to the capsid polypeptide and/or the affinity polypeptide and wherein the affinity polypeptide is expressed on the surface of the engineered capsid. In some embodiments, the affinity polypeptide is capable of specifically binding to, specifically associating with, or otherwise specifically interacting with a predetermined target. Exemplary predetermined targets are described in greater detail elsewhere herein, such as with respect to the genetically encoded affinity molecule.

The engineered phagemids can produce one or more components of the engineered bacteriophages (e.g., an affinity molecule, engineered capsid, and/or translated sequencing molecule) as well as be cargo inside said engineered bacteriophages that can then be associated with a cell and/or nucleus to which the engineered bacteriophage specifically binds, associates, or otherwise interacts with (see e.g. FIGS. 1A-1C). As previously described, the affinity molecule can be produced from a genetically encoded affinity molecule on an engineered phagemid. In some embodiments, the affinity molecule is encoded by a polynucleotide on an engineered phagemid (i.e., the genetically encoded affinity molecule) as previously described. In some embodiments, the affinity molecule comprises a peptide, polypeptide, polynucleotide, or a combination thereof In some embodiments, the affinity molecule is an engineered scaffold. Exemplary engineered scaffolds, such as engineered protein scaffolds are described in greater detail elsewhere herein, such as with respect to the genetically encoded affinity molecules. In some embodiments, the affinity molecule is an antibody or fragment thereof. Antibodies and fragments thereof are described in greater detail elsewhere herein, such as with respect to the genetically encoded affinity molecules.

In some embodiments, the engineered bacteriophage includes a capsid polypeptide that is incorporated into the capsid (also referred to as a coat) of an engineered bacteriophage that is produced from a genetically encoded capsid polypeptide on the engineered phagemid. In some embodiments the capsid polypeptide is encoded by a polynucleotide on an engineered phagemid (i.e., the genetically encoded capsid polypeptide) as previously described.

In some embodiments, the capsid polypeptide is a major capsid polypeptide. In some embodiments, the capsid polypeptide is an engineered minor capsid polypeptide.

In some embodiments, the engineered bacteriophage includes, such as in its capsid, one or more, such as 1, 2, 3, 4, 5, 6, 7, 8, 9, 10 or more, capsid polypeptides. In some embodiments, a translated sequencing molecule is fused to or is operatively coupled to one or more of the one or more capsid polypeptides. In some embodiments, the capsid polypeptides are homogenous. In some embodiments, the capsid polypeptides are heterogenous.

The capsid polypeptide can be any suitable or based upon any suitable bacteriophage capsid polypeptide. In some embodiments, the bacteriophage capsid polypeptide(s) is/are or includes a lysogenic bacteriophage capsid polypeptide. In some embodiments, the bacteriophage capsid polypeptide(s) is/are or includes a genetically encoded lytic bacteriophage capsid polypeptide. In some embodiments, the bacteriophage capsid polypeptide(s) is/are or includes a Caudovirale and/or Ligamenvirales bacteriophage capsid polypeptide. In some embodiments, the bacteriophage capsid polypeptide(s) is/are or includes an Ackermannviridae, Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttaviridae, Inoviridae, Leviviridae, Microviridae, Plasmaviridae, Pleolipoviridae, Portogloboviridae, Sphaerolipoviridae, Spiraviridae, Tectiviridae, Tristromaviridae, Turriviridae, and combinations thereof.

In some embodiments, the capsid polypeptide is a M13 phage capsid polypeptide. In some embodiments, the M13 phage capsid polypeptide is a P3, P6, P7, P8, or P9 genetically encoded capsid polypeptide. In some embodiments, the capsid polypeptide is a λ phage capsid polypeptide.

In some embodiments, the engineered bacteriophage includes a translated sequencing molecule (also referred to herein as a sequencing molecule polypeptide). In some embodiments, the sequencing molecule polypeptide is fused to or operatively coupled to the capsid polypeptide and/or the affinity polypeptide and wherein the affinity polypeptide is expressed on the surface of the engineered capsid.

Methods of generating engineered bacteriophages are generally known in the art and can be used to generate the engineered bacteriophages described herein. Exemplary methods and techniques for generating the engineered bacteriophages are demonstrated in the Working Examples herein and discussed in e.g., Piers et al., 2016. Microbiol. Molc. Biol. Rev. 80(3):523-543; Chen et al, 2019. Front. Microbiol. 10: Article 954, https://doi.org/10.3389/fmicb.2019.00954 (particularly at pages 2-5); Brown et al., 2017. Quant. Biol. 5(1): 42-54 (particularly at 23-28), which are incorporated by reference herein as if expressed in their entireties and can be adapted for use with the phagemids and bacteriophages described herein.

### Engineered Display Construct and Display System Libraries

Described in certain embodiments herein are display construct libraries comprising: a plurality of engineered display constructs according to any one of the preceding paragraphs or as elsewhere described herein.

In certain example embodiments, the display constructs are engineered phagemids.

In certain example embodiments, two or more engineered display constructs comprise a unique genetically encoded affinity molecule, a unique genetically encoded display molecule, a unique genetically encoded sequencing molecule, or a combination thereof.

In certain example embodiments, each of the engineered display constructs comprise a unique genetically encoded affinity molecule, a unique genetically encoded display molecule, a unique genetically encoded sequencing molecule, or any combination thereof.

Described in certain example embodiments herein are pluralities of engineered display constructs comprising an engineered display construct library as in any one of the preceding paragraphs or as elsewhere described herein.

In some embodiments, a selected pool of engineered display constructs and or engineered display systems can be generated via a selection method. In some embodiments, the selected pool includes engineered display constructs and/or engineered display systems that can contain an affinity molecule that can target a specific or desired target molecule that is selected by a user or the system. Described in certain embodiments herein are methods of generating a specific pool of engineered display constructs or engineered display systems having a desired target affinity, comprising (a) generating an input display construct or engineered display system library, wherein each display construct or display system present in the input library is as in any one of the preceding paragraphs or as elsewhere described herein; (b) removing from the input library via negative selection at least some of the engineered display constructs or engineered display systems in the input library that do not specifically bind or otherwise associate with a desired target; (c) positively selecting engineered display constructs or engineered display systems form the pool formed after step (b) that specifically bind or otherwise associate with the desired target, (d) amplifying the positively selected engineered display constructs or engineered display systems.

In certain example embodiments, the method further comprises repeating steps (b) through (c) or through (d) one or more times, wherein the input for step (b) is the output from step (c) or step (d).

In certain example embodiments, the method further comprises sequencing one or more regions of the positively selected engineered display constructs.

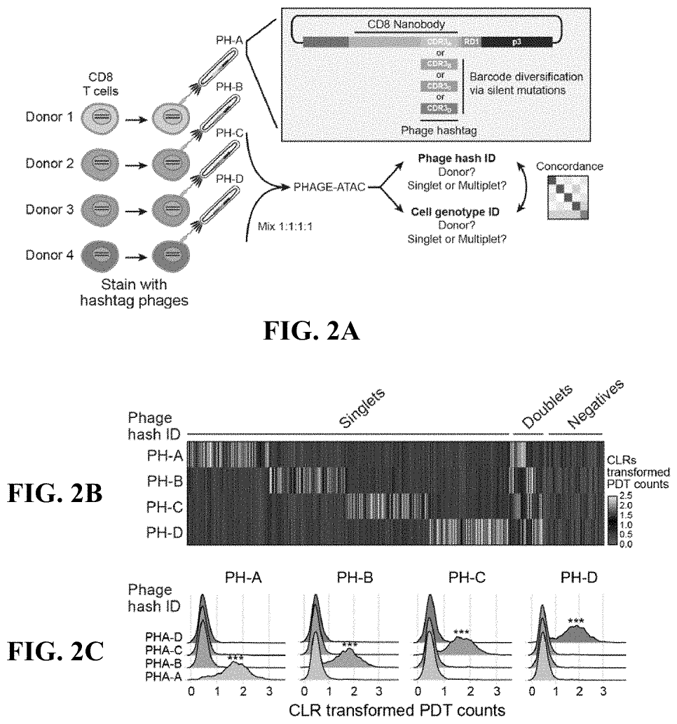

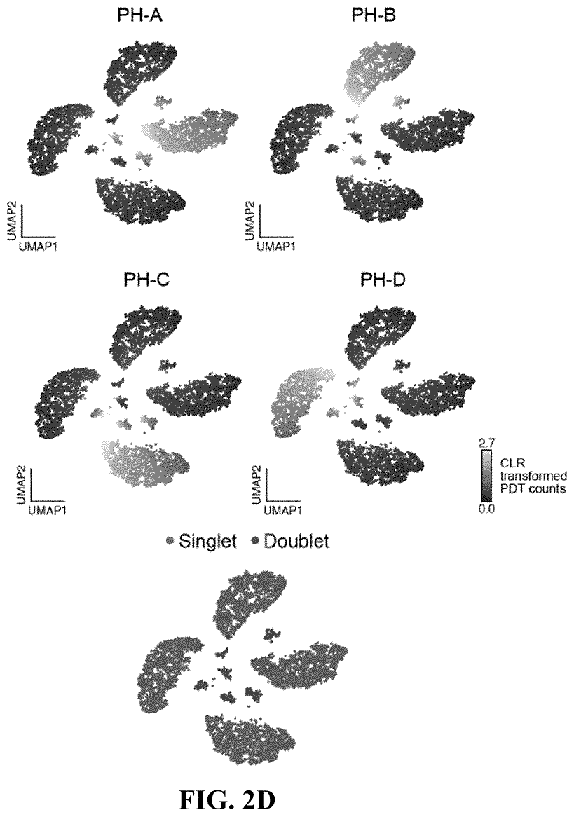

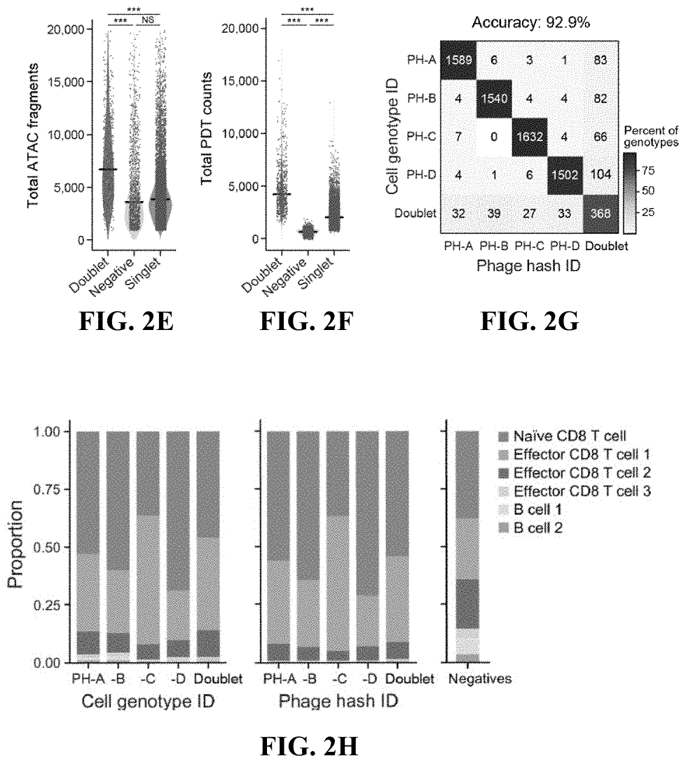

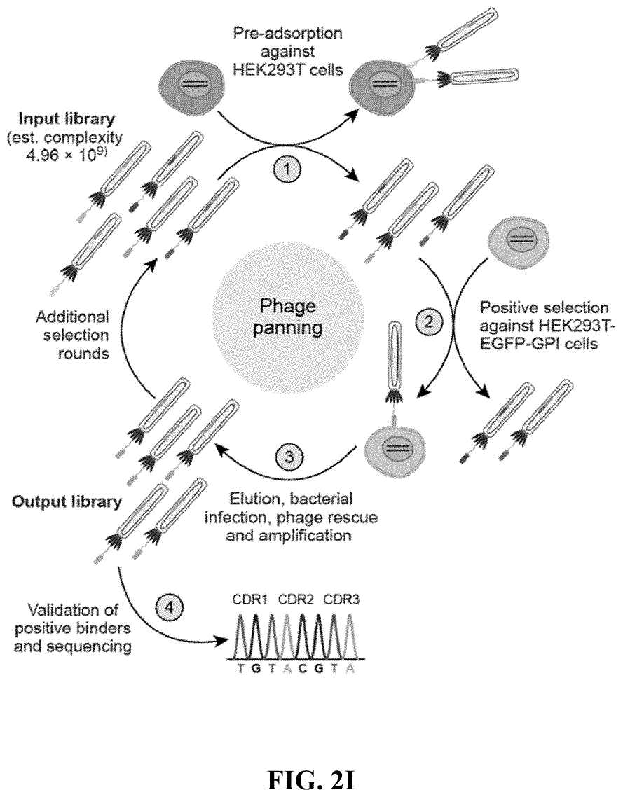

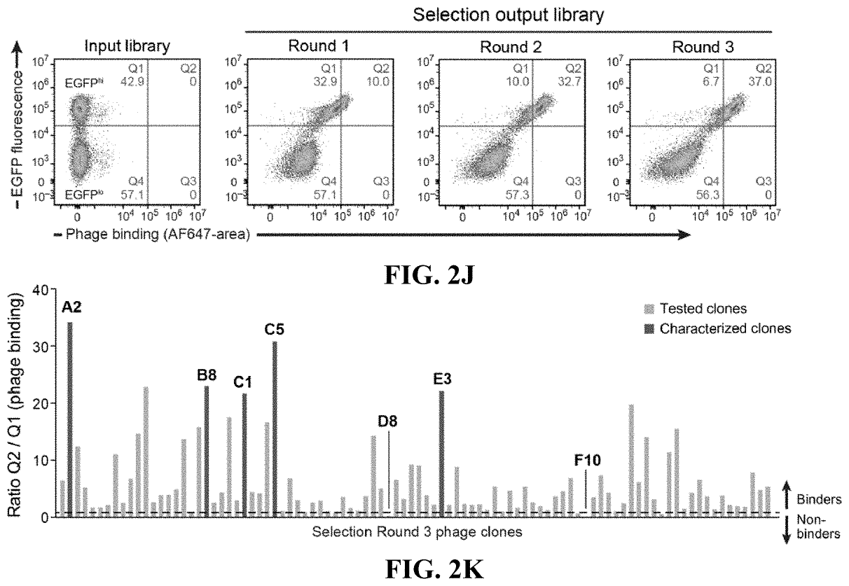

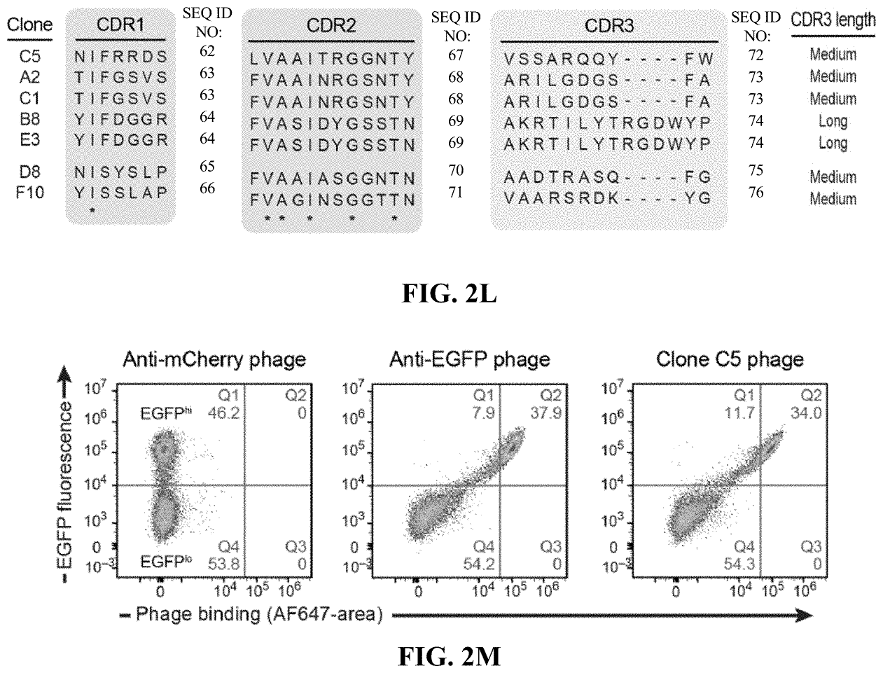

An exemplary method for generating a pool of specific engineered display constructs or systems in the context of phagemids and bacteriophages is shown in FIG. 2I.

Described in embodiments herein are display construct libraries (such as phagemid libraries) that are composed of a plurality of engineered display constructs (such as phagemids) as described in greater detail elsewhere herein. The library can contain 2 to 1000 or more phagemids, such as 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 10,000, or 100,000 or more.

In some embodiments, the plurality of engineered display constructs (e.g., phagemids) are heterogenous in at least one or more of the following: the genetically encoded affinity molecule, the genetically encoded sequencing molecule, and/or the genetically encoded display molecule (e.g., capsid polypeptide). In some embodiments, the plurality of phagemids are homogenous in at least one or more of the following: the genetically encoded affinity molecule, the genetically encoded sequencing molecule, and/or the genetically encoded display molecule (e.g., capsid polypeptide).

In some embodiments, two or more engineered display constructs (e.g., phagemids) comprise a unique genetically encoded affinity molecule, a unique genetically encoded capsid molecule, a unique genetically encoded sequencing molecule, or a combination thereof. In some embodiments, each of the display constructs (e.g., phagemids) comprise a unique genetically encoded affinity molecule, a unique genetically encoded display (e.g., capsid polypeptide) molecule, a unique genetically encoded sequencing molecule, or any combination thereof.

Also described herein are engineered display system (e.g., bacteriophage) libraries that can include a plurality of engineered display systems (e.g., bacteriophages) described in greater detail elsewhere herein. In some embodiments, a plurality of engineered display system (e.g., bacteriophage) includes a plurality of engineered display constructs (e.g., phagemids). In some embodiments, one or more of the engineered display systems (e.g., bacteriophages) of the plurality of engineered display systems (e.g., bacteriophages) can each include one or a plurality of engineered display constructs (e.g., phagemids).

The engineered display system (e.g., bacteriophage) library can contain 2 to 1000 or more bacteriophages, such as 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 10,000, or 100,000 or more.

In some embodiments the plurality of engineered display systems (e.g., bacteriophages) are heterogenous in at least one or more of the following: an affinity molecule, a sequencing molecule polypeptide, a display molecule (e.g., capsid polypeptide), or a combination thereof. In some embodiments, the plurality of engineered display system (e.g., bacteriophages) are homogenous in at least one or more of the following: the affinity molecule, the sequencing molecule polypeptide, and/or the display molecule (e.g., capsid polypeptide).

In some embodiments, two or more engineered display system (e.g., bacteriophages) comprise a unique affinity molecule, a unique display molecule (e.g., capsid polypeptide), a unique sequencing molecule polypeptide, or a combination thereof. In some embodiments, each of the engineered display construct (e.g., phagemid) comprise a unique genetically encoded affinity molecule, a unique genetically encoded display molecule (e.g., capsid polypeptide) (e.g., a unique genetically encoded sequencing molecule polypeptide, or any combination thereof.

### Kits

Any of the compounds, compositions, formulations, particles, cells, described herein or a combination thereof can be presented as a combination kit. As used herein, the terms “combination kit” or “kit of parts” refers to the compounds, compositions, formulations, particles, cells and any additional components that are used to package, sell, market, deliver, use, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are used and/or administered simultaneously, the combination kit can contain the agents in a single formulation or in separate formulations. When the compounds, compositions, formulations, particles, and cells described herein or a combination thereof and/or kit components are not administered or used simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface. The instructions can provide information regarding the content of the compounds, compositions, formulations, particles, cells, described herein or a combination thereof contained therein, safety information regarding the content of the compounds, compositions, formulations, particles, and cells described herein or a combination thereof contained therein, information regarding the dosages, indications for use, and/or recommended treatment regimen(s) for the compound(s) and/or formulations contained therein.

In some embodiments, the kit includes one or more engineered display constructs (e.g., phagemids) described in greater detail elsewhere herein. In some embodiments, the kit includes one or more engineered display construct libraries (e.g., phagemid libraries) and/or engineered display system libraries (e.g. bacteriophage libraries) as described in greater detail elsewhere herein. In some embodiments, the kit includes one or more engineered display systems (e.g., bacteriophages) described in greater detail elsewhere herein. In some embodiments, the kit includes a plurality of engineered display systems (e.g., bacteriophages) described in greater detail elsewhere herein. In some embodiments, the instructions include directions for multi-omic analysis using the engineered display constructs (e.g., phagemid(s)), engineered display systems (e.g. bacteriophages), and/or libraries/plurality thereof that are present in the kit. In some embodiments, the instructions include directions for performing a method of multi-omic analysis as described elsewhere herein.

In some embodiments, a kit for multi-omic analysis includes an engineered display construct (e.g., phagemid), an engineered display construct library (e.g., a phagemid library), and/or an engineered display system (e.g. bacteriophage), engineered display system library (e.g. bacteriophage library) or plurality thereof described elsewhere herein. In some embodiments, the affinity molecule of each engineered display system, (e.g., bacteriophage) is capable of specifically binding a predetermined target present on the surface of and/or inside of a cell and/or nucleus. In some embodiments, the genetically encoded affinity molecule is capable of generating an affinity molecule polypeptide capable of specifically binding a predetermined target present on the surface of and/or inside of a cell and/or nucleus.

In some embodiments, the predetermined target is a microorganism protein; a cancer-associated protein; an immune checkpoint inhibitor; a cell-type marker; a cell-state marker; f) a non-cancer disease or condition biomarker; or a combination thereof.

Exemplary predetermined targets are described in greater detail elsewhere herein, such as with respect to the genetically encoded affinition molecule.

In some examples, the system and kits may comprise cell fixation reagents, DNA tagmentation reagents (e.g., transposase), RT-PCR reagents (e.g., primers for reverse transcription), devices and/or reagents for performing split-pool barcoding, devices and/or reagents for sequencing and sequence reads analysis, or any combination thereof.

### Methods of Multi-Omic Analysis

The engineered display constructs (e.g., phagemids) and engineered display systems (e.g., bacteriophages) can be used in multi-omic analysis. Described in certain example embodiments herein are methods of multi-omic single cell or single nuclei analysis, comprising: (a) specifically binding one or more individual cells, individual nuclei, or both with an engineered display system or plurality thereof of as in any one of the preceding paragraphs or as described elsewhere herein; (b) allowing each affinity molecule to specifically bind a target molecule present inside of and/or on the surface of the one or more individual cells and/or individual nuclei; (c) fixing the specifically bound engineered display system(s) to the one or more individual cells and/or individual nuclei; (d) accessing cellular polynucleotides within one or more individual specifically bound cells and/or individual specifically bound nuclei; e) accessing the engineered display construct(s) in the specifically bound engineered display construct(s); and f) characterizing one or more features of the one or more individual specifically bound cells and/or individual specifically bound nucleic based, at least in part, on sequencing, in whole or in part, (i) the accessed genetically encoded affinity molecule, genetically encoded sequencing molecule, or both present in the specifically bound engineered display construct and (ii) the one or more accessed cellular and/or nuclear polynucleotides.

In certain example embodiments, the method further comprises generating, within one or more individual specifically bound cells and/or nuclei, cDNA copies of cellular RNA molecules.

In certain example embodiments, characterizing one or more features is based, at least in part, on sequencing the cDNA copies of cellular RNA molecules.

In certain example embodiments, sequencing comprises sequencing a portion of the accessed genetically encoded affinity molecule, genetically encoded sequencing molecule, or both present in the specifically bound engineered display construct and a portion of each of the one or more accessed cellular and/or nuclear polynucleotides.

In certain example embodiments, the step of accessing polynucleotides present inside the individual cell and/or individual nuclei comprises permeabilizing the cell, permeabilizing the nucleus, lysing the cells, lysing the nucleus or a combination thereof.

In certain example embodiments, the method further comprises tagmenting, within individual cells and/or individual nuclei, genomic DNA to produced tagmented genomic DNA fragments.

In certain example embodiments, sequencing comprises sequencing the one or more tagmented genomic DNA fragments or a portion thereof.

In certain example embodiments, the method further comprises incorporating a cell or nuclei barcode into the one or more cellular polynucleotides, cDNA copies, tagmented genomic DNA fragments, the genetically encoded affinity molecule, the genetically encoded sequencing molecule, or a combination thereof, such that the one or more cellular polynucleotides, cDNA copies, tagmented genomic DNA fragments, genetically encoded affinity molecule, the genetically encoded sequencing molecule, or a combination thereof from the same cell receive the same unique cell and/or from the same nuclei receive the same nuclei barcode sequence.

In certain example embodiments, the method further comprises incorporating into the one or more cellular polynucleotides, cDNA copies, tagmented genomic DNA fragments, the genetically encoded affinity molecule, the genetically encoded sequencing molecule, or a combination thereof comprises one or more barcodes; one or more PCR handles; one or more unique molecular identifiers (UMIs); one or more affinity tags; one or more sequencing adapters; one or more linkers; a poly(T) sequence; a poly(A) sequence; one or more primer sites; or any combination thereof.

In certain example embodiments, the method further comprises amplifying the one or more cellular polynucleotides, nuclear polynucleotides, cDNA copies, tagmented genomic DNA fragments, the genetically encoded affinity molecule, the genetically encoded sequencing molecule, or a combination thereof.

In certain example embodiments, the method further comprises mixing the one or more cellular polynucleotides, cDNA copies, tagmented genomic DNA fragments, the genetically encoded affinity molecule, the genetically encoded sequencing molecule, or a combination thereof with an oligonucleotide-adorned bead, wherein each oligonucleotide on the oligonucleotide-adorned bead comprises: one or more linkers; one or more barcodes; one or more unique molecular identifiers (UMIs); one or more affinity tags; one or more sequencing adapters one or more reaction handles or substrates; one or more primer sites; a poly(T) sequence; a poly(A) sequence; one or more PCR handles; or any combination thereof.

In certain example embodiments, the method further comprises isolating a cell and/or nucleus that is specifically bound to and fixed to one or more engineered bacteriophages in or on a substrate, in an individual discrete volume, or container.

In certain example embodiments, the substrate or individual discrete volume is a liquid, a solid, a semi-solid, or a gel.

In certain example embodiments, the substrate or individual discrete volume is a droplet or a slide.

In certain example embodiments, the container is a well, microwell, capillary, or microcapillary.

In certain example embodiments, mixing with an oligonucleotide-adorned bead occurs in or on the substrate or container.

In certain example embodiments, one or more oligonucleotide-adorned beads are present on a surface of the substrate or container and are arranged in an ordered array, wherein each oligonucleotide-adorned bead has a unique barcode corresponding to the x,y coordinate of the oligonucleotide-adorned bead in the array.

In certain example embodiments, the method further comprises depositing a tissue section comprising the one or more individual cells on the ordered array.

In certain example embodiments, the one or more individual cells are present in a tissue sample and specific binding and fixing occurs in situ.

In certain example embodiments, sequencing the genetically encoded affinity molecule, the genetically encoded sequencing molecule, or both and sequencing the one or more cellular polynucleotides, one or more nuclear polynucleotides, or both occurs in situ.

In certain example embodiments, the method further comprises converting unmethylated cytosines to uracil in the genomic DNA via bisulfite conversion prior to sequencing the genomic DNA or portion thereof.

In certain example embodiments, the one or more features comprise a cellular RNA expression profile; a surface protein expression profile; an epigenetic feature of a genomic DNA region in the cell; or a combination thereof.

In certain example embodiments, the epigenetic feature comprises: a profile of chromatin accessibility along the genomic DNA region; a DNA binding protein occupancy for a binding site in the genomic DNA region; a nucleosome-free DNA in the genomic DNA region; a positioning of the nucleosomes along the genomic DNA region; methylation status; chromatin states; or a combination thereof.

In certain example embodiments, sequencing comprises a single cell, single nucleus sequencing technique, or both.

In some embodiments, the engineered display constructs, engineered display systems, engineered phagemids and engineered bacteriophages are used to simultaneously provide genomic, epigenomic, transcriptomic, protein expression, or a combination thereof information on one or more cells and/or nuclei. In some embodiments, the engineered phagemids and engineered bacteriophages are used to simultaneously provide genomic, epigenomic, transcriptomic, protein expression, or a combination thereof information on a single cell or single nucleus.

In some embodiments, a method of multi-omic analysis includes specifically binding one or more individual cells, individual nuclei, or both with an engineered display system (e.g. an engineered bacteriophage) or plurality thereof of as described in greater detail elsewhere herein; allowing each affinity molecule to specifically bind a target molecule present inside of and/or on the surface of the one or more individual cells and/or individual nuclei; fixing the specifically bound engineered display system(s) (e.g., engineered bacteriophage(s)) to the one or more individual cells and/or individual nuclei; accessing cellular polynucleotides within one or more individual specifically bound cells and/or individual specifically bound nuclei accessing the engineered display system(s) (e.g. engineered phagemid(s)) in the specifically bound engineered bacteriophage(s); and characterizing one or more features of the one or more individual specifically bound cells and/or individual specifically bound nucleic based, at least in part, on sequencing, in whole or in part, (i) the accessed genetically encoded affinity molecule, genetically encoded sequencing molecule, or both present in the specifically bound phagemid and (ii) the one or more accessed cellular and/or nuclear polynucleotides.

In some embodiments, a method of multi-omic analysis includes generating, within one or more individual specifically bound cells and/or nuclei, cDNA copies of cellular and/or nuclear RNA molecules.

In some embodiments, characterizing one or more features is based, at least in part, on sequencing the cDNA copies of cellular and/or nuclear RNA molecules.

In some embodiments, sequencing comprises sequencing a portion or entirety of the accessed genetically encoded affinity molecule, genetically encoded sequencing molecule, or both present in the specifically bound engineered phagemid and sequencing a portion or entirety of each of the one or more accessed cellular and/or nuclear polynucleotides.

In some embodiments, accessing polynucleotides present inside the individual cell and/or individual nuclei comprises permeabilizing the cell, permeabilizing the nucleus, lysing the cells, lysing the nucleus or a combination thereof. Suitable techniques of accessing polynucleotides and/or nucleus within a cell are demonstrated in the Working Examples herein and are also generally known in the art.

In some embodiments, the method can include assaying for transposase-accessible chromatin (ATAC) or steps thereof to assess chromatin accessibility. In some embodiments, the method of multi-omic analysis described herein includes tagmenting, within individual cells and/or individual nuclei, genomic DNA to produce tagmented genomic DNA fragments. In some embodiments, sequencing comprises sequencing the one or more tagmented genomic DNA fragments or a portion thereof.

In some embodiments, a method of multi-omic analysis includes incorporating a cell or nuclei barcode into the one or more cellular polynucleotides, cDNA copies, tagmented genomic DNA fragments, the genetically encoded affinity molecule, the genetically encoded sequencing molecule, or a combination thereof, such that the one or more cellular polynucleotides, cDNA copies, tagmented genomic DNA fragments, genetically encoded affinity molecule, the genetically encoded sequencing molecule, or a combination thereof from the same cell receive the same unique cell and/or from the same nuclei receive the same nuclei barcode sequence.

In some embodiments, a method of multi-omic analysis includes incorporating into the one or more cellular polynucleotides, cDNA copies, tagmented genomic DNA fragments, the genetically encoded affinity molecule, the genetically encoded sequencing molecule, or a combination thereof one or more barcodes; one or more PCR handles; one or more unique molecular identifiers (UMIs); one or more affinity tags; one or more sequencing adapters; one or more linkers; a poly(T) sequence; a poly(A) sequence; one or more primer sites; or any combination thereof.

### Amplification of Nucleic Acids

In some embodiments, a method of multi-omic analysis includes amplifying the one or more cellular polynucleotides, nuclear polynucleotides, cDNA copies, tagmented genomic DNA fragments, the genetically encoded affinity molecule, the genetically encoded sequencing molecule, or a combination thereof. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM). In certain embodiments, the amplification can utilize a transposase-based isothermal amplification method (see e.g. WO 2020/006049, which is incorporated by reference herein as if expressed in its entirety), nickase-based isothermal amplification method (see e.g. WO 2020/006067, which is incorporated by reference herein as if expressed in its entirety), a helicase-based amplification method (see e.g. WO 2020/006036, which is incorporated by reference herein as if expressed in its entirety), polymerase chain reaction (PCR), quantitative real-time PCR; reverse transcriptase PCR (RT-PCR); real-time PCR (rt PCR); real-time reverse transcriptase PCR (rt RT-PCR); nested PCR; strand displacement amplification; transcription-free isothermal amplification; ligase chain reaction amplification; gap filling ligase chain reaction amplification; coupled ligase detection and PCR; or other methods known in the art. In some embodiments, amplification is via LAMP. In some embodiments, amplification is via RPA.

In certain example embodiments, the RNA or DNA amplification is nucleic acid sequence-based amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH₄)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or apatamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In certain embodiments, detection of DNA with the methods or systems of the invention requires transcription of the (amplified) DNA into RNA prior to detection.

In some embodiments, the end joined nucleic acids or other nucleic acids are selectively amplified. In some examples, to selectively amplify the end joined nucleic acids, a 3′ DNA adaptor and a 5′ RNA, or conversely a 5′ DNA adaptor and a 3′ RNA adaptor can be ligated to the ends of the molecules can be used to mark the end joined nucleic acids. Using primers specific for these adaptors only end joined nucleic acids may be amplified during an amplification procedure such as PCR. In some embodiments, the target end joined nucleic acid is amplified using primers that specifically hybridize to the adapter nucleic acid sequences present at the 3′ and 5′ ends of the end joined nucleic acids. In some embodiments, the non-ligated ends of the nucleic acids are end repaired. In some embodiments attaching sequencing adapters to the ends of the end ligated nucleic acid fragments. The amplification may be performed with primers with one or more barcodes.

In some embodiments, a method of multi-omic analysis includes mixing the one or more cellular polynucleotides, cDNA copies, tagmented genomic DNA fragments, the genetically encoded affinity molecule, the genetically encoded sequencing molecule, or a combination thereof with an oligonucleotide-adorned bead or surface, wherein each oligonucleotide on the oligonucleotide-adorned bead or surface comprises: one or more linkers; one or more barcodes; one or more unique molecular identifiers (UMIs); one or more affinity tags; one or more sequencing adapters one or more reaction handles or substrates; one or more primer sites; a poly(T) sequence; a poly(A) sequence; one or more PCR handles; or any combination thereof.

In some embodiments, a method of multi-omic analysis includes isolating a cell and/or nucleus that is specifically bound to and fixed to one or more engineered bacteriophages in or on a substrate, in an individual discrete volume, or container. In some embodiments, the substrate or individual discrete volume is a liquid, a solid, a semi-solid, or a gel. In some embodiments, the substrate or individual discrete volume is a droplet or a slide. In some embodiments, the container is a well, microwell, capillary, or microcapillary. In some embodiments, the substrate and/or container are optically transparent. In some embodiments, the substrate and/or container are optically opaque. In some embodiments, mixing with an oligonucleotide-adorned bead occurs in or on the substrate or container.

In some embodiments, the oligonucleotides adorning the bead include one or more barcodes, index sequences, linkers, capture barcodes, or other barcodes, UMIs, or combinations thereof. In some embodiments, each of the oligonucleotides adorning a bead includes a bead-specific barcode or UMI.

### Discrete Volumes

As used herein, a “discrete volume” or “discrete space” may refer to a container, receptacle, or other defined volume or space that can be defined by properties that prevent and/or inhibit migration of molecules, particles and/or nucleic acid containing specimens. For example, a discrete volume or space may be defined by physical properties such as walls of a discrete well, tube, or surface of a droplet which may be impermeable or semipermeable. The discrete volume or space may also refer to a reaction unit or region within a larger volume, where that region is not defined by walls but rather is defined spatially by location within the larger volume. For example, the discrete volume or space may be chemically defined, diffusion rate limited defined, electro-magnetically defined, or optically defined, or any combination thereof. By “diffusion rate limited” is meant volumes or spaces that are only accessible to certain species or reactions because diffusion constraints that would effectively limit the migration of a particular molecule, particle, or nucleic acid containing specimen from one discrete volume to another. By “chemically defined” is meant a volume or space where only certain molecules, particles, or nucleic acid containing specimens can exist because of their chemical or molecular properties. For example, certain gel beads may exclude certain molecules, particles, or nucleic acid containing specimens from entering the beads but not others by surface charge, matrix size, or other physical property of the gel bead. By “electro-magnetically defined” is meant volumes or spaces where the electro-magnetic properties of certain molecules, particles, or cells may be used to define certain volumes or spaces. For example, by capturing magnetic particles within a magnetic field or directly by magnets. By “optically defined” is meant volumes or spaces that may be defined by illuminating the volume or space with visible, ultraviolet, infrared, or other wavelengths of light such that only target molecules within the defined space or volume are detected.

### Droplets

In some cases, an individual discrete volume is in a droplet. The present disclosure enables high throughput and high-resolution delivery of reagents to individual emulsion droplets that may contain cells, organelles, nucleic acids, proteins, etc. through the use of monodisperse aqueous droplets that are generated by a microfluidic device as a water-in-oil emulsion. The droplets may be carried in a flowing oil phase and stabilized by a surfactant. In one aspect, single cells or single organelles or single nuclei or single molecules (proteins, RNA, DNA) are encapsulated into uniform droplets from an aqueous solution/dispersion. In a related aspect, multiple cells or multiple nuclei or multiple molecules may take the place of single cells or single nuclei or single molecules. The aqueous droplets of volume ranging from 1 pL to 10 nL work as individual reactors. Disclosed embodiments provide 104 to 105 single cells in droplets which can be processed and analyzed in a single run.

To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination.

Each species of droplet may be introduced at a confluence point in a main microfluidic channel from separate inlet microfluidic channels. In some cases, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length may be selected such that faster species of droplets catch up to the slowest species. Size constraints of the channel may prevent the faster moving droplets from passing the slower moving droplets resulting in a train of droplets entering a merge zone. Multi-step chemical reactions, biochemical reactions, or assay detection chemistries may involve a fixed reaction time before species of different type may be added to a reaction. Multi-step reactions may be achieved by repeating the process multiple times with a second, third or more confluence points each with a separate merge point. Highly efficient and precise reactions and analysis of reactions may be achieved when the frequencies of droplets from the inlet channels are matched to an optimized ratio and the volumes of the species are matched to provide optimized reaction conditions in the combined droplets.

Fluidic droplets may be screened or sorted within a fluidic system of the invention by altering the flow of the liquid containing the droplets. For instance, in some embodiments, a fluidic droplet may be steered or sorted by directing the liquid surrounding the fluidic droplet into a first channel, a second channel, etc. In certain embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels may be controlled to direct the droplet selectively into one of the channels, and changes in pressure can be affected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. In another embodiment, the expansion and/or contraction of the liquid reservoir may be combined with other flow-controlling devices and methods, e.g., as described herein. Non-limiting examples of devices able to cause the expansion and/or contraction of a liquid reservoir include pistons.

Key elements for using microfluidic channels to process droplets include: (1) producing droplet of the correct volume, (2) producing droplets at the correct frequency and (3) bringing together a first stream of sample droplets with a second stream of sample droplets in such a way that the frequency of the first stream of sample droplets matches the frequency of the second stream of sample droplets, preferably, bringing together a stream of sample droplets with a stream of premade library droplets in such a way that the frequency of the library droplets matches the frequency of the sample droplets.

Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions, e.g., a library may contain plurality of different compounds at a range of concentrations encapsulated as separate library elements for screening their effect on cells or enzymes, alternatively a library could be composed of a plurality of different primer pairs encapsulated as different library elements for targeted amplification of a collection of loci, alternatively a library could contain a plurality of different antibody species encapsulated as different library elements to perform a plurality of binding assays. The introduction of a library of reaction conditions onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second. However, in practice droplets pack with oil between them that slowly drains. Over time the carrier fluid drains from the library droplets and the number density of the droplets (number/mL) increases. Hence, a simple fixed rate of infusion for the drive fluid does not provide a uniform rate of introduction of the droplets into the microfluidic channel in the substrate. Moreover, library-to-library variations in the mean library droplet volume result in a shift in the frequency of droplet introduction at the confluence point. Thus, the lack of uniformity of droplets that results from sample variation and oil drainage provides another problem to be solved. For example, if the nominal droplet volume is expected to be 10 pico-liters in the library, but varies from 9 to 11 pico-liters from library-to-library then a 10,000 pico-liter/second infusion rate will nominally produce a range in frequencies from 900 to 1,100 droplet per second. In short, sample to sample variation in the composition of dispersed phase for droplets made on chip, a tendency for the number density of library droplets to increase over time and library-to-library variations in mean droplet volume severely limit the extent to which frequencies of droplets may be reliably matched at a confluence by simply using fixed infusion rates. In addition, these limitations also have an impact on the extent to which volumes may be reproducibly combined. Combined with typical variations in pump flow rate precision and variations in channel dimensions, systems are severely limited without a means to compensate on a run-to-run basis. The foregoing facts not only illustrate a problem to be solved, but also demonstrate a need for a method of instantaneous regulation of microfluidic control over microdroplets within a microfluidic channel.

Combinations of surfactant(s) and oils must be developed to facilitate generation, storage, and manipulation of droplets to maintain the unique chemical/biochemical/biological environment within each droplet of a diverse library. Therefore, the surfactant and oil combination must (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition to the requirements on the droplet library function and stability, the surfactant-in-oil solution must be coupled with the fluid physics and materials associated with the platform. Specifically, the oil solution must not swell, dissolve, or degrade the materials used to construct the microfluidic chip, and the physical properties of the oil (e.g., viscosity, boiling point, etc.) must be suited for the flow and operating conditions of the platform.

Droplets formed in oil without surfactant are not stable to permit coalescence, so surfactants must be dissolved in the oil that is used as the continuous phase for the emulsion library. Surfactant molecules are amphiphilic—part of the molecule is oil soluble and part of the molecule is water soluble. When a water-oil interface is formed at the nozzle of a microfluidic chip for example in the inlet module described herein, surfactant molecules that are dissolved in the oil phase adsorb to the interface. The hydrophilic portion of the molecule resides inside the droplet and the fluorophilic portion of the molecule decorates the exterior of the droplet. The surface tension of a droplet is reduced when the interface is populated with surfactant, so the stability of an emulsion is improved. In addition to stabilizing the droplets against coalescence, the surfactant should be inert to the contents of each droplet, and the surfactant should not promote transport of encapsulated components to the oil or other droplets.

A droplet library may be made up of a number of library elements that are pooled together in a single collection (see, e.g., US Patent Publication No. 2010002241). Libraries may vary in complexity from a single library element to 1015 library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” are also referred to herein as an “emulsion library” or “emulsion libraries.” These terms are used interchangeably throughout the specification.

### Solid Support

In some embodiments, an individual discrete volume is on a solid support. A solid support may be a bead or micro-bead, or a plurality of micro-beads, micro-arrays, micro-wells, or micro-lids. The solid support can be shaped in any manner required for an end use application and may have a shape that is circular, square, star, or porous. Examples of suitable solid supports include, but are not limited to, inert polymers (preferably non-nucleic acid polymers), beads, glass, or peptides. In some embodiments, the solid support is an inert polymer or a bead. The bead is a silica bead, a hydrogel bead or a magnetic bead. In some embodiments, the solid support comprises a magnetic core. Examples of suitable polymers include a hydroxylated methacrylic polymer, a hydroxylated poly(methyl methacrylate), a polystyrene polymer, a polypropylene polymer, a polyethylene polymer agarose, or cellulose. In one example, the solid support may be wells in a microwell plate. In another example, the solid support may be particles, e.g., beads.

In cases where the solid support is particles, the solid support has an average particle size between about 10 microns to 200 microns, about 10 microns to 190 microns, about 10 microns to 180 microns, about 10 microns to 170 microns, about 10 microns to 160 microns, about 10 microns to 150 microns, about 10 microns to about 140 microns, about 10 to about 130 microns, about 10 to about 120 microns, about 10 microns to about 110 microns, about 10 microns to about 100 microns, about 10 microns to about 90 microns, about 10 microns to about 80 microns, about 10 microns to about 70 microns, about 10 microns to about 60 microns, about 10 microns to about 50 microns, about 10 microns to about 40 microns, about 10 microns to 30 microns, about 10 microns to about 20 microns, about 20 microns to about 30 microns, about 20 microns to about 40 microns, about 20 microns to about 50 microns, about 20 microns to about 60 microns, about 20 microns to about 70 microns, about 20 microns to about 80 microns, about 20 microns to about 100 microns, about 20 microns to about 100 microns, about 50 microns to about 100 microns, about 100 microns to 200 microns, or about 30 microns. In some embodiments, the bead or micro-bead has an average size, measured as average diameter of 20-40 μm.

In some embodiments, the solid support may be functionalized, e.g., to permit covalent attachment of the agent and/or label. Such functionalization on the support may comprise reactive groups that permit covalent attachment to an agent and/or a label.

### Microfluidic Devices

In some embodiments, the discrete volume is contained in a microfluidic device. Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of the one or more flow channels and the array of microwells. The substrate material is poured into a mold and allowed to set to create a stamp. The stamp is then sealed to a solid support such as, but not limited to, glass.

Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

The microfluidic devices may further comprise inlet and outlet ports, or openings, which in turn may be connected to valves, tubes, channels, chambers, and syringes and/or pumps for the introduction and extraction of fluids into and from the microfluidic device. The microfluidic devices may be connected to fluid flow actuators that allow directional movement of fluids within the microfluidic device. Example actuators include, but are not limited to, e.g., syringe pumps, mechanically actuated recirculating pumps, electroosmotic pumps, bulbs, bellows, diaphragms, or bubbles intended to force movement of fluids.

### Features of Discrete Volumes

The slip steps may comprise splitting a sample into a number of discrete volumes, e.g., in at least 2, at least 4, at least 6, at least 8, at least 10, at least 20, at least 30, at least 40, at least 50, at least 60, at least 70, at least 80, at least 90, at least 100, at least 200, at least 300, at least 400, or at least 500 discrete volumes.

Each discrete volume may have a suitable number of cells or nuclei for the number of barcodes available to avoid excessive barcode collision. For example, the number of cells in each volume and the number of barcodes available may be used to reach a barcode collision rate less than 10%, 9%, 8%, 7%, 6%, 5%, 4%, 3%, 2%, or 1%. In one example, the collision rate may be less than 5%. In another example, the barcode collision rate may be less than 1%.

### Spatial Detection

In some embodiments, the method of multi-omic analysis described herein can include spatial detection of genomic, epigenomic, transcriptomic, and/or proteomic information of a population of cells, tissues and/or organisms. In some embodiments, one or more oligonucleotide-adorned beads are present on a surface of the substrate or container and are arranged in an ordered array, wherein each oligonucleotide-adorned bead has a unique barcode corresponding to the x,y coordinate of the oligonucleotide-adorned bead in the array. In some embodiments, the method further includes depositing a tissue section comprising the one or more individual cells on the ordered array. In some embodiments, the one or more individual cells are present in a tissue sample and specific binding and fixing occurs in situ. In some embodiments, sequencing the genetically encoded affinity molecule, the genetically encoded sequencing molecule, or both and sequencing the one or more cellular polynucleotides, one or more nuclear polynucleotides, or both occurs in situ.

### Fixing Cells

In some embodiments, the methods herein may comprise the optional step of fixing cells. After fixation, the molecules (e.g., nucleic acids) in the cells may be fixed in positions relative to each other. The fixation may be performed by crosslinking. When nucleic acids are cross-linked, either directly, or indirectly, and the information about spatial relationships between the different nucleic acid fragments in the cell, or cells, is maintained during this joining step herein, and substantially all of the end joined nucleic acid fragments formed at this step were in spatial proximity in the cell prior to the crosslinking step. Therefore, at this point the information about which sequences are in spatial proximity to other sequences in the cell is locked into the end joined fragments. In some cases, the methods comprise holding the nucleic acids in a fixed position relative to one another prior to fragmenting. The nucleic acids may be held in the fixed position by crosslinking the cells or nuclei in the cells or isolated nuclei from the cells.

The fixation may be performed by chemical crosslinking, for example, by contacting the cells or isolated nuclei in the cells with one or more chemical cross linkers. In some embodiments, the cells are fixed, for example with a fixative, such as an aldehyde, for example formaldehyde or glutaraldehyde. In some embodiments, a sample of one or more cells is cross-linked with a cross-linker to maintain the spatial relationships in the cell. For example, a sample of cells can be treated with a cross-linker to lock in the spatial information or relationship about the molecules in the cells, such as the DNA and RNA in the cell.

In some embodiments, the relative positions of the nucleic acid can be maintained without using crosslinking agents. For example, the nucleic acids can be stabilized using spermine and spermidine (see Cullen et al., Science 261, 203 (1993), which is specifically incorporated herein by reference in its entirety). Other methods of maintaining the positional relationships of nucleic acids are known in the art. In some embodiments, nuclei are stabilized by embedding in a polymer such as agarose. In some embodiments, the cross-linker is a reversible cross-linker. In some embodiments, the cross-linker is reversed, for example after the fragments are joined. In specific examples, the nucleic acids are released from the cross-linked three-dimensional matrix by treatment with an agent, such as a proteinase, that degrade the proteinaceous material form the sample, thereby releasing the end ligated nucleic acids for further analysis, such as determination of the nucleic acid sequence. In specific embodiments, the sample is contacted with a proteinase, such as Proteinase K.

In some embodiments of the disclosed methods, the cells are contacted with a crosslinking agent to provide the cross-linked cells. In some examples, the cells are contacted with a protein-nucleic acid crosslinking agent, a nucleic acid-nucleic acid crosslinking agent, a protein-protein crosslinking agent or any combination thereof. By this method, the nucleic acids present in the sample become resistant to special rearrangement and the spatial information about the relative locations of nucleic acids in the cell is maintained. In some examples, a cross-linker is a reversible, such that the cross-linked molecules can be easily separated in subsequent steps of the method. In some examples, a cross-linker is a non-reversible cross-linker, such that the cross-linked molecules cannot be easily separated. In some examples, a cross-linker is light, such as UV light. In some examples, a cross linker is light activated.

Examples of cross-linkers include formaldehyde, paraformaldehyde, alcohol (e.g., methanol), disuccinimidyl glutarate, UV light, psoralens and their derivatives such as aminomethyltrioxsalen, glutaraldehyde, ethylene glycol bis[succinimidyl succinate], bissulfosuccinimidyl suberate, 1-Ethyl-[3-dimethylaminopropyl]carbodiimide (EDC) bis[sulfosuccinimidyl] suberate (BS³) and other compounds known to those skilled in the art, including those described in the Thermo Scientific Pierce Crosslinking Technical Handbook, Thermo Scientific (2009) as available on the world wide web at piercenet.com/files/1601673 _Crosslink_HB_Intl.pdf, or may involve embedding cells or tissue in a paraffin wax or polyacrylamide support matrix.

In some embodiments, it is not necessary to hold the nucleic acids in place using a chemical fixative or crosslinking agent. Thus, in some embodiments, no crosslinking agent is used. In still other embodiments, the nucleic acids are held in position relative to each other by the application of non-crosslinking means, such as by using agar or other polymer to hold the nucleic acids in position.

### Reversing the Crosslinking

In some embodiments, the methods may also comprise reversing the crosslinking at some point. In some examples, the crosslinking may be reversed prior to the nucleic acid shearing, bisulfite treatment, and/or nucleic acid isolation. Reverse crosslinking may be performed by incubating the cells, nuclei, or molecules with detergents (e.g., SDS), proteinase (e.g., proteinase K), and/or at high temperature (e.g., at least 60° C., 70° C., 80° C., or 90° C., such as about 68° C.).

### Cell Lysis and Permeabilization

In some embodiments, the cells are lysed to release the cellular contents, for example after crosslinking. In some cases, the cells are lysed and nuclei are released before nucleic acid fragmentation. In some examples, the nuclei are lysed as well. In other examples, the nuclei are maintained intact, which can then be isolated and optionally lysed, for example using an reagent that selectively targets the nuclei or other separation technique known in the art. In some examples, the sample comprises permeabilized nuclei, multiple nuclei, isolated nuclei, synchronized cells, (such at various points in the cell cycle, for example metaphase) or acellular. In some embodiments, the nucleic acids present in the sample are purified, for example using ethanol precipitation. In example embodiments of the disclosed method the cells and/or cell nuclei are not subjected to mechanical lysis. In some example embodiments, the sample is not subjected to RNA degradation. In specific embodiments, the sample is not contacted with an exonuclease to remove of biotin from un-ligated ends. In some embodiments, the sample is not subjected to phenol/chloroform extraction. In certain embodiments, the cells or nuclei may be permeabilized to allow reagents for processing nucleic acids to contact the nucleic acids.

### Nucleic Acid Shearing

In some embodiments, the end-joined or other nucleic acid fragments may be sheared to fragments of suitable sizes for further processing. For example, the sheared fragments may have a length from about 100 bp to about 1000 bp, from about 200 bp to about 800 bp, from about 300 bp to about 600 bp, from about 300 bp to about 500 bp, from about 200 bp to about 400 bp, from about 250 bp to about 450 bp, from about 350 bp to about 550 bp, from about 250 bp to about 350 bp, from about 300 bp to about 400 bp, from about 350 bp to about 450 bp, from about 400 bp to about 500 bp, from about 450 bp to about 550 bp, or from about 500 bp to about 600 bp.

In some examples, the shearing may be performed by passing the nucleic acid through a narrow capillary or orifice, for example a hypodermic needle, sonication, such as by ultrasound, grinding in cell homogenizers, for example stirring in a blender, or nebulization. In an example, the nucleic acid is sheared by sonication, e.g., using an ultrasonicator.

### Attaching Adapters

The methods may further comprise attaching one or more adapters to the isolated nucleic acids from a cell or nuclei. The adapters may comprise binding sites for primers (e.g., sequence primers, amplification primers, etc.), barcodes, and other elements facilitating nucleic acid analysis and processing. The adapters may be attached to the nucleic acids using ligase or primer extension.

In some cases, the isolated nucleic acids are single stranded DNA. In these cases, one or more adapters may be attached to one end of the single stranded DNA. The adapter(s) may be attached to the 3′ end of the single stranded DNA. In certain cases, the adapter(s) may be attached to the 5′ end of the single stranded DNA. In some cases, both ends of the single stranded DNA may be attached with adapter(s). The adapters may be single stranded.

In some cases, a second strand of DNA may be synthesized using the isolated single stranded DNA, e.g., by primer extension. One or more adapters may be attached to the second strand. The adapter(s) may be attached to the 3′ end of the second strand. In certain cases, the adapter(s) may be attached to the 5′ end of the second strand. In some cases, both ends of the second strand may be attached with adapter(s).

### Detectable Features

In some embodiments of a method of multi-omic analysis described herein, the one or more features comprise a cellular or nuclear RNA expression profile; a surface protein expression profile; an epigenetic feature of a genomic DNA region in the cell; or a combination thereof. In some embodiments, the epigenetic feature comprises: a profile of chromatin accessibility along the genomic DNA region; a DNA binding protein occupancy for a binding site in the genomic DNA region; a nucleosome-free DNA in the genomic DNA region; a positioning of the nucleosomes along the genomic DNA region; methylation status; chromatin states; or a combination thereof.

As used herein “expression profile” is used interchangeable with “expression signature”. As used herein, the term “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g. differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. A signature can be composed of any number of genes, proteins epigenetic elements, and/or combinations thereof. For example, a gene signature may include a list of genes differentially expressed in a distinction of interest. The signature can be composed completely of or contain 1, 2, 3, 4, 5, 6, 7, 8, 9, 10, 11, 12, 13, 14, 15, 16, 17, 18, 19, or 20 or more genes, proteins and/or epigenetic elements. In aspects, the signature can be composed completely of or contain 1-20 or more, 2-20 or more, 3-20 or more, 4-20 or more, 5-20 or more, 6-20 or more, 7-20 or more, 8-20 or more, 9-20 or more, 10-20 or more, 11-20 or more, 12-20 or more, 13-20 or more, 14-20 or more, 15-20 or more, 16-20 or more, 17-20 or more, 18-20 or more, 19-20 or more, or 20 or more genes, proteins and/or epigenetic elements.

### Lysing Cells in Beads

In certain example embodiments, sequencing DNA or other polynucleotide for each cell comprises lysing the cell in each bead such that genomic DNA is retained in the polymerized bead, releasing the beads from the outer capsule and re-encapsulating the beads in a second outer capsule, the second outer capsule comprising genomic DNA amplification reagents. The beads are maintained under conditions sufficient for genomic DNA amplification. The beads are then released from the second capsule and re-encapsulated in a third capsule comprising tagmentation reagents to generate genomic fragments, the tagmentation reagents comprising transposomes loaded with sequencing adapters. The sequencing adapters may further comprise a unique origin specific barcode or unique combination of origin specific barcodes. After maintaining the encapsulated beads under conditions sufficient for tagmentation, the tagmented DNA is then isolated to prepare a DNA sequencing library comprising the genomic DNA fragments. The genomic DNA library is then sequenced to determine a genotype for each microscale biological system. In certain example embodiments, the DNA amplification reagents are multiple displacement amplification reagents (MDA). In certain example embodiments, the method may further comprise a DNA sequencing library amplification step prior to the sequencing step. In certain example embodiments, the DNA sequencing library amplification step comprises releasing each encapsulated bead into a separate individual discrete volume comprising DNA amplification reagent, breaking the bead to release the genomic DNA fragments labeled with sequencing adapters, and optionally origin-specific barcodes, and maintaining the separate individual discrete volumes under conditions sufficient to allow for DNA amplification. In certain example embodiments, the amplification step may further comprise addition of a second barcode to each genomic DNA fragment.

In certain example embodiments, the contents of the bead, the outer capsule, or both may be altered over the time-course of a given assay. In certain example embodiments, the contents are altered by contacting the double encapsulated microscale biological system with one or more reagents that are diffusible into the outer shell and/or bead. The one or more reagents may be used to sustain replication or growth of the microscale biological system or determine an additional biological function of the microscale biological system. In certain other example embodiments, altering the contents may comprise releasing the beads from the first outer capsule and re-encapsulating the beads in an additional outer capsule. The process of releasing and re-encapsulating the beads to introduce additional agents may be repeated over multiple iterations as needed per assay design. In addition, the beads may be sorted, for example, based on the readout of a reporter element, between each iteration of release and re-encapsulation. In some embodiments, a reporter element described herein may produce an optically detectable signal. In one embodiment, the reporter element comprises magnetic-based separation, and may comprise labeling a biological molecule of interest with a magnetic particle and isolating the biological molecule of interest using the magnetic particles. In another embodiment, the biological molecule of interest is selected from the group consisting of a protein, a cell surface marker, and a nucleic acid, or combinations thereof.

In some embodiments, the method includes fragmenting the genomic DNA, cDNA, or other polynucleotide in a cell or nucleus. In some embodiments, fragmenting is performed by digesting the nucleic acids using a nuclease. In some embodiments, the nuclease is methylation insensitive. In some embodiments, such as where a bisulfite technique is utilized, the nuclease is methylation insensitive. In some embodiments, the method further comprises, prior to the bisulfite treatment, shearing the nucleic acids. In some embodiments, the sheared nucleic acids have a length from about 300 base pairs (bp) to about 500 bp.

### Fragmenting Nucleic Acids

The methods herein may comprise fragmenting nucleic acids. In some embodiments, in order to create discrete portions of nucleic acid that can optionally be joined together in subsequent steps of the methods, the nucleic acids present in the cells, such as cross-linked cells, are fragmented. In one example embodiment, the fragmentation may be done enzymatically. In another example embodiment, the fragmentation may be done chemically.

### Overhanging Ends

For example, DNA can be fragmented using an enzyme (e.g., an endonuclease) that cuts a specific sequence of DNA and leaves behind a DNA fragment with an overhang, thereby yielding fragmented DNA.

When a nuclease cleaves DNA asymmetrically a stretch of single stranded nucleotides is left. In some cases, the overhang is a 5′ overhang. In certain cases, the overhang is a 3′ overhang. In other examples an endonuclease can be selected that cuts the DNA at random spots and yields overhangs or blunt ends. In some embodiments, fragmenting the nucleic acid present in the one or more cells comprises enzymatic digestion with an endonuclease that leaves 5′ overhanging ends. Enzymes that fragment, or cut, nucleic acids and yield an overhanging sequence are known in the art and can be obtained from such commercial sources as New England BioLabs® and Promega®. One of ordinary skill in the art will appreciate that using different fragmentation techniques, such as different enzymes with different sequence requirements, will yield different fragmentation patterns and therefore different nucleic acid ends. The process of fragmenting the sample can yield ends that are capable of being joined.

In some examples, the endonuclease for nucleic acid fragmentation is a methylation-sensitive endonuclease. A “methylation-sensitive endonuclease” refers to a restriction enzyme that cleaves at or in proximity to an unmethylated recognition sequence but does not cleave at or in proximity to the same sequence when the recognition sequence is methylated. Exemplary 5′-methyl cytosine sensitive endonuclease include, e.g., Aat II, Aci I, Acl L Age L Alu L Asc L Ase I, AsiS I, Bbe I, BsaA I, BsaH I, BsiE I, BsiW I, BsrF I, BssH II, BssK I, BstB I, BstN I, BstU I, Cla I, Eae I, Eag I, Fau I, Fse I, Hha I, HinP1 I, HinC II, Hpa II, Hpy99 I, HpyCH4 IV, Kas I, Mlu I, MapAl I, MboI, Msp I, Nae I, Nar I, Not I, Pml I, Pst I, Pvu I, Rsr II, Sac II, Sap I, Sau3A I, Sfl I, Sfo I, SgrA I, Sma I, SnaB I, Tsc I, Xma I, or Zra I. In one example, the endonuclease used herein is MboI.

In some examples, the endonuclease for nucleic acid fragmentation is a methylation-dependent endonuclease. A “methylation-dependent endonuclease” refers to a restriction enzyme that cleaves at or near a methylated recognition sequence but does not cleave at or near the same sequence when the recognition sequence is not methylated. Methylation-dependent endonuclease can recognize, for example, specific sequences comprising a methylated-cytosine or a methylated-adenosine. Methylation-dependent restriction enzymes include those that cut at a methylated recognition sequence (e.g., DpnI) and enzymes that cut at a sequence that is not at the recognition sequence (e.g., McrBC). Exemplary methylation-dependent endonucleases include, e.g., McrBC, McrA, MrrA, and Dpn I. One of skill in the art will appreciate that homologs and orthologs of the restriction enzymes described herein are also suitable for use in the present invention.

In some examples, the endonuclease for nucleic acid fragmentation is a methylation insensitive endonuclease. A “methylation insensitive endonuclease” refers to a restriction enzyme that cuts DNA regardless of the methylation state of the base of interest (A or C) at or near the recognition sequence. In some examples, the endonuclease for nucleic acid fragmentation is a methylation sensing endonuclease. A “methylation sensing endonuclease” refers to a restriction enzyme whose activity changes in response to the methylation of its recognition sequence.

### Filling in Overhangs

The methods may further comprise filling in the overhangs in the fragmented nucleic acids. The overhangs may be filled in with nucleotides using a polymerase (e.g., a DNA polymerase). In some cases, the filled in nucleic acid fragments are blunt ended at the filled end (e.g., 5′ end).

### End Joining

The methods herein may further comprise joining the ends of the fragmented nucleic acids. In some embodiments, the fragmented nucleic acids are end joined at the filled in ends, for example, by ligation using a nucleic acid ligase (e.g., T4 ligase), or otherwise attached to another fragment that is in close physical proximity. The ligation, or other attachment procedure, for example nick translation or strand displacement, creates one or more end joined nucleic acid fragments having a junction, for example a ligation junction, wherein the site of the junction, or at least within a few bases, includes one or more labeled nucleic acids, for example, one or more fragmented nucleic acids that have had their overhanging ends filled and joined together. While this step typically involves a ligase, it is contemplated that any means of joining the fragments can be used, for example any chemical or enzymatic means. Further, it is not necessary that the ends be joined in a 3′-5′ ligation.

The joined ends may create a junction, which is a site where two nucleic acid fragments or joined, for example using the methods described herein. A junction may contain information about the proximity of the nucleic acid fragments that participate in formation of the junction. For example, junction formation between to nucleic acid fragments indicates that these two nucleic acid sequences where in close proximity when the junction was formed, although they may not be in proximity in liner nucleic acid sequence space. Thus, a junction can define ling range interactions. In some embodiments, a junction is labeled, for example with a labeled nucleotide, for example to facilitate isolation of the nucleic acid molecule that includes the junction.

The end joined nucleic acid fragments may have be between about 100 and about 1000 bases in length, although longer and shorter fragments are also contemplated. In some embodiments, the nucleic acid fragments are from about 100 to about 1000 bases in length, such as about 100, about 150, about 200, about 250, about 300, about 350, about 400, about 450, about 500, about 550, about 600, about 650, about 700, about 750, about 800, about 850, about 900, about 950 or about 1000 bases in length, for example form about 100 to about 1000, form about 200 to about 800, form about 500 to about 850, form about 100 to about 500 and form about 300 to about 775 base pairs in length and the like. In specific examples, end joined fragments are selected for sequence determination that are form about 300 to 500 base pairs in length.

Treating with Bisulfite

The methods may further comprise treating the nucleic acids (e.g., the end joined nucleic acid fragments) with an agent that modifies unmethylated base the nucleic acids. In some embodiments, such treatment (e.g., bisulfite treatment) allows the discrimination between unmethylated and methylated base. In some cases, the agent modifies unmethylated cytosine, e.g., the agent alters the chemical composition of unmethylated cytosine but does not change the chemical composition of methylated cytosine. For example, the agent may selectively modifies either the methylated or non-methylated form of CpG dinucleotide.

In some examples, the agent that modifies unmethylated base is sodium bisulfite. Sodium bisulfite comprises sodium hydrogen sulfite having the chemical formula of NaHSO₃. Sodium bisulfite may function to deaminate cytosine into uracil; but does not affect 5-methylcytosine (a methylated form of cytosine with a methyl group attached to carbon 5). When the bisulfite-treated DNA is amplified via polymerase chain reaction, the uracil is amplified as thymine and the methylated cytosine is amplified as cytosine. Suitable chemical reagents include hydrazine and bisulphite ions and the like. In some examples, when treating DNA, sodium bisulfite converts unmethylated cytosine to uracil, while methylated cytosines are maintained. Without wishing to be bound by a theory, it is understood that sodium bisulfite reacts readily with the 5,6-double bond of cytosine, but poorly with methylated cytosine. Cytosine reacts with the bisulfite ion to form a sulfonated cytosine reaction intermediate that is susceptible to deamination, giving rise to a sulfonated uracil. The sulfonated group can be removed under alkaline conditions, resulting in the formation of uracil. The nucleotide conversion results in a change in the sequence of the original DNA. The resulting uracil has the base pairing behavior of thymine, which differs from cytosine base pairing behavior. To that end, uracil is recognized as a thymine by DNA polymerase. In some cases, after PCR or sequencing, the resultant product contains cytosine only at the position where 5-methylcytosine occurs in the starting template DNA.

In some examples, the treatment (e.g., bisulfite treatment) may be performed prior to nucleic acid isolation (e.g., by capture agents). In some examples, the treatment may be performed prior to any adapter ligation step. In some examples, the treatment may be performed prior to nucleic acid amplification. In some examples, the treatment (e.g., bisulfite treatment) may be performed prior to nucleic acid isolation, adapter ligation, and nucleic acid amplification. In these cases, the negative effects from harsh chemical conditions during the treatment may be avoided in the following nucleic acid isolation, adapter ligation, and nucleic acid amplification steps. In certain examples, it is also contemplated that the treatment step is performed after nucleic acid isolation, adapter ligation, and/or nucleic acid amplification steps.

### Determining Sequences

Nucleic acids may be analyzed using various methods, including determining the sequences of the junctions or a portion thereof may be determined. The sequence reads may provide physical proximity information of nucleic acids. Such information may be used to determine spatial proximity relationships (e.g., in situ) of the nucleic acids in cells. In some cases, determining the spatial proximity relationships between the nucleic acids comprises identifying chromosomal location of nucleic acid sequences at 5′, 3′ or both 5′ and 3′ of the junctions. Advantageously, the methods allow for simultaneous determining of spatial proximity between nucleic acids and the methylation profile of the nucleic acids.

In some embodiments, the epigenetic profile, e.g., methylation profile, of the junctions or sequences close to the junctions may be determined. In some cases, determining the methylation profile comprises generating a genome-wide methylation profile of cells of interest. The relationship between the spatial proximity and the epigenetic (e.g., methylation) profile of the nucleic acids may be determined. Such relationship may be correlated with a disease, and thus may be used for diagnosing and/or developing a treatment plan for the disease. In some examples, the nucleic acid analysis comprises quantifying a frequency with which pairs of loci in the nucleic acids are found adjacent, and/or a frequency with which loci in the nucleic acids are methylated.

### Sequencing

The methods herein may further include sequencing one or more nucleic acids processed by the steps herein. For example, after barcoded and isolated, the genomic DNA, cDNA, the barcode sequence(s), and a portion thereof, may be sequenced.

Generally, the sequencing can be performed using automated Sanger sequencing (AB13730xl genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®); Moleculo sequencing (see Voskoboynik et al. eLife 2013 2:e00569 and U.S. patent application Ser. No. 13/608,778, filed Sep. 10, 2012); DNA nanoball sequencing; Single molecule real time (SMRT) sequencing; Nanopore DNA sequencing; Sequencing by hybridization; Sequencing with mass spectrometry; and Microfluidic Sanger sequencing. Examples of information that can be obtained from the disclosed methods and the analysis of the results thereof, include without limitation uni- or multiplex, 3 dimensional genome mapping, genome assembly, one dimensional genome mapping, the use of single nucleotide polymorphisms to phase genome maps, for example to determine the patterns of chromosome inactivation, such as for analysis of genomic imprinting, the use of specific junctions to determine karyotypes, including but not limited to chromosome number alterations (such as unisomies, uniparental disomies, and trisomies), translocations, inversions, duplications, deletions and other chromosomal rearrangements, the use of specific junctions correlated with disease to aid in diagnosis. As would be apparent, forward and reverse sequencing primer sites that are compatible with a selected next generation sequencing platform can be added to the ends of the fragments during the amplification step. In certain embodiments, the fragments may be amplified using PCR primers that hybridize to the tags that have been added to the fragments, where the primer used for PCR have 5′ tails that are compatible with a particular sequencing platform. In certain cases, the primers used may contain a molecular barcode (an “index”) so that different pools can be pooled together before sequencing, and the sequence reads can be traced to a particular sample using the barcode sequence.

In some cases, the sequencing may be next generation sequencing. The terms “next-generation sequencing” or “high-throughput sequencing” refer to the so-called parallelized sequencing-by-synthesis or sequencing-by-ligation platforms currently employed by Illumina, Life Technologies, and Roche, etc. Next-generation sequencing methods may also include nanopore sequencing methods or electronic-detection based methods such as Ion Torrent technology commercialized by Life Technologies or single-molecule fluorescence-based method commercialized by Pacific Biosciences. Any method of sequencing known in the art can be used before and after isolation. In certain embodiments, a sequencing library is generated and sequenced.

At least a part of the processed nucleic acids and/or barcodes attached thereto may be sequenced to produce a plurality of sequence reads. The fragments may be sequenced using any convenient method. For example, the fragments may be sequenced using Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol Biol. 2009; 553:79-108); Appleby et al (Methods Mol Biol. 2009; 513:19-39) and Morozova et al (Genomics. 2008 92:255-64), which are incorporated by reference for the general descriptions of the methods and the particular steps of the methods, including all starting products, methods for library preparation, reagents, and final products for each of the steps. As would be apparent, forward and reverse sequencing primer sites that are compatible with a selected next generation sequencing platform can be added to the ends of the fragments during the amplification step. In certain embodiments, the fragments may be amplified using PCR primers that hybridize to the tags that have been added to the fragments, where the primer used for PCR have 5′ tails that are compatible with a particular sequencing platform. In certain cases, the primers used may contain a molecular barcode (an “index”) so that different pools can be pooled together before sequencing, and the sequence reads can be traced to a particular sample using the barcode sequence.

In some embodiments the sequencing technique incorporates a bead, such as an oligo adorned bead. Oligo adorned beads are described in greater detail elsewhere herein.

In some cases, the sequencing may be performed at certain “depth.” The terms “depth” or “coverage” as used herein refers to the number of times a nucleotide is read during the sequencing process. In regards to single cell RNA sequencing, “depth” or “coverage” as used herein refers to the number of mapped reads per cell. Depth in regards to genome sequencing may be calculated from the length of the original genome (G), the number of reads (N), and the average read length (L) as N×L/G. For example, a hypothetical genome with 2,000 base pairs reconstructed from 8 reads with an average length of 500 nucleotides will have 2× redundancy.

In some cases, the sequencing herein may be low-pass sequencing. The terms “low-pass sequencing” or “shallow sequencing” as used herein refers to a wide range of depths greater than or equal to 0.1× up to 1×. Shallow sequencing may also refer to about 5000 reads per cell (e.g., 1,000 to 10,000 reads per cell).

In some cases, the sequencing herein may deep sequencing or ultra-deep sequencing. The term “deep sequencing” as used herein indicates that the total number of reads is many times larger than the length of the sequence under study. The term “deep” as used herein refers to a wide range of depths greater than 1× up to 100×. Deep sequencing may also refer to 100× coverage as compared to shallow sequencing (e.g., 100,000 to 1,000,000 reads per cell). The term “ultra-deep” as used herein refers to higher coverage (>100-fold), which allows for detection of sequence variants in mixed populations.

### Analysis of Sequence Reads

Sequence reads obtained using methods herein may be analyzed, e.g., for characterizing one or more features of the cells, tissues, or subject from which the nucleic acid molecules are from or derived from.

In some embodiments, the sequence reads may be analyzed for determining one or more epigenetic features in genomic DNA, expression profiles of one or more genes, or a combination thereof. In some examples, the sequence reads may comprise sequence information of different types of nucleic acids, e.g., genomic DNA and cDNA. In such cases, the sequence reads may be analyzed for determining a correlation of one or more epigenetic features and expression profiles of one or more genes in the same cell. The sequence reads of nucleic acids from or derived from the same cell may be identified using the unique barcode sequence described herein.

The epigenetic features may include a profile of chromatin accessibility along a region of interest, DNA binding protein (e.g., transcription factors) occupancy for a site in the region, nucleosome-free DNA in the region, positioning of nucleosomes along the region, a profile of chromatin states along the region, global occupancy of a binding site for the DNA binding protein by, e.g., aggregating data for one DNA binding protein over a plurality of sites to which that protein binds. Information about the sequence analyzed may also be obtained. Such information may include the positions of promoters, introns, exons, known enhancers, transcriptional start sites, untranslated regions, terminators, etc.

The term “chromatin accessibility,” as used herein, refers to how accessible a nucleic acid site is within a polynucleotide, such as in genomic DNA, e.g., how “open” the chromatin is. A nucleic acid site associated with a polypeptide, such as with genomic DNA in nucleosomes, is usually inaccessible. A nucleic acid site not complexed with a polypeptide is generally accessible, such as with genomic DNA between nucleosomes (with the exception of nucleic acid sites complexed with transcription factors and other DNA binding proteins). The term “DNA binding protein occupancy,” as used herein, refers to whether a binding site for a sequence specific DNA binding protein (e.g., a binding site for a transcription factor) is occupied by the DNA binding protein. DNA binding protein occupancy can be measured quantitatively or qualitatively. The term “global occupancy,” as used herein, refers to whether a plurality of different binding sites for a DNA binding protein that are distributed throughout the genome (e.g., a binding site for a transcription factor) are bound by the DNA binding protein. DNA binding protein occupancy can be measured quantitatively or qualitatively.

The epigenetic features may be analyzed in the context with the sequence information. The epigenetic features may provide information regarding active regulatory regions and/or the transcription factors that are bound to the regulatory regions. For example, nucleosome positions may be inferred from the lengths of sequencing reads generated. Alternatively and additionally, transcription factor binding sites may be inferred from the size, distribution and/or position of the sequencing reads generated. In some cases, novel transcription factor binding sites may be inferred from sequencing reads generated. In other cases, novel transcription factors can be inferred from sequencing reads generated.

In some embodiments, the correlation between the epigenetic feature(s) of a region of interest and the expression profile of one or more genes in the region may be obtained. The expression profile may be obtained using sequence reads of cDNA or RNA transcribed from the one or more genes.

The methods may be used for performing any assays that involve analyzing nucleic acids. In some embodiments, the methods may be used for determining chromatin accessibility or chromatin remodeling. In these cases, the methods, the methods may be used for identifying and analyzing molecules in or derived from open chromatin regions. In some embodiments, the methods may be used for performing whole genome sequencing. For example, for performing whole genome sequencing, the methods may comprise pretreating cells with detergents (e.g., SDS), and depleting nucleosome (e.g., using Lithium Assisted Nucleosome Depletion (LAND)). In some examples, the nucleosome depletion may be performed as described in Vitak S A et al., Sequencing thousands of single-cell genomes with combinatorial indexing, Nat Methods. 2017 March; 14(3): 302-308.

In some embodiments, sequencing comprises a single cell or component thereof, single nucleus sequencing technique or component thereof, or both. Exemplary single cell and single nucleus include, but are not limited to, Act-Seq (see e.g. Wu Y. E. et al. (2017) Neuron 96(2): 313-329); CEL-Seq (see e.g., Hashimshony T. et al. (2012) Cell Rep 2: 666-673); CirSeq (see e.g., Acevedo A. et al. (2014) Nature 505: 686-690); CITE-Seq (see e.g., Stoeckius M., et al. (2017) Nat Methods 14(9): 865-868); CLaP (see e.g., Binan L. et al. (2016) Nat Commun 7: 11636); CRISPR-UMI (see e.g., Michlits G. et al. (2017) Nat Methods 14(12): 1191-1197); CROP-Seq (see e.g., Datlinger P. et al. (2017) Nat Methods 14(3): 297-301); CytoSeq (see e.g., Fan H. C. et al. (2015) Science 347: 1258367); Digital RNA (see e.g., Shiroguchi K. et al. (2012) Proc Natl Acad Sci USA 109:1347-1352); Dip-C (see e.g., Tan L., et al. (2018) Science 361(6405): 924-928); Div-Seq (see e.g., Habib N. et al. (2016) Science 353(6302): 925-928); DP-Seq (see e.g., Bhargava V. et al. (2013) Sci Rep 3: 1740); DroNC-seq (see e.g., Habib N. et al. (2017) Nat Methods 14(10): 955-958); Drop-Seq (see e.g., Macosko E. Z. et al. (2015) Cell 161: 1202-1214); DR-Seq (see e.g., Dey S. S. et al. (2015) Nat Biotechnol 33: 285-9); Drop-ChIP (see e.g., Rotem A. et al. (2015) Nat Biotechnol 33: 1165-72); Duplex-Seq (see e.g., Schmitt M. W. et al. (2012) Proc Natl Acad Sci USA 109: 14508-14513); ECCITE-seq (see e.g., Mimitou E. P. et al. (2019) Nat Methods 16(5): 409-412); FREQ-Seq (see e.g., Chubiz L. M. et al. (2012) PLoS One 7: e47959); FRISCR (see e.g., Thomsen E. R. et al. (2016) Nat Methods 13: 87-93); G&T-seq (see e.g., Macaulay I. C. et al. (2015) Nat Methods 12: 519-522); HiRes-Seq (see e.g., Imashimizu M. et al. (2013) Nucleic Acids Res 41:9090-9104); Hi-SCL (see e.g., Rotem A. et al. (2015) PLoS One 10: e0116328); IMS-MDA (see e.g., Seth-Smith H. M. et al. (2013) Nat Protoc 8: 2404-2412); inDrop (see e.g., Klein A. M. et al. (2015) Cell 161: 1187-201); LIANTI (see e.g., Chen C. et al. (2017) Science 356(6334): 189-194); MALBAC (see e.g., Zong C. et al. (2012) Science 338: 1622-1626); MARS-seq (see e.g., Jaitin D. A. et al. (2014) Science 343:776-9); MATQ-seq (see e.g., Sheng K. et al. (2017) Nat Methods 14(3): 267-270); MDA (see e.g., Dean F. B. et al. (2001) Genome Res 11: 1095-1099); Microwell-seq (see e.g., Han X. et al. (2018) Cell 172(5): 1091-1107.e1017); MIDAS (see e.g., Gole J. et al. (2013) Nat Biotechnol 31:1126-32); MIPSTR (see e.g., Carlson K. D. et al. (2015) Genome Res 25: 750-761); Mosaic-seq (see e.g., Han X. et al. (2018) Cell 172(5): 1091-1107 e1017); MULTI-seq (see e.g., McGinnis C. S. et al. (2019) Nat Methods 16(7): 619-626); NanoCAGE (see e.g., Plessy C. et al. (2010) Nat Methods 7: 528-534); Nanogrid SNRS (see e.g., Gao R. et al. (2017) Nat Commun 8(1): 228); nuc-seq (see e.g., Wang Y. et al. (2014) Nature 512: 155-160); Nuc-Seq/SNES (see e.g., Leung M. L. et al. (2015) Genome Biology 16(1): 55); OS-Seq (see e.g., Myllykangas S. et al. (2011) Nat Biotechnol 29: 1024-1027); PAIR (see e.g., Bell T. J. et al. (2015) Methods Mol Biol 1324: 457-68); Quartz-Seq (see e.g., Sasagawa Y. et al. (2013) Genome Biol 14: R31); Quartz-Seq2 (see e.g., Sasagawa Y. et al. (2018) Genome Biology 19(1): 29); RamDA-seq (see e.g., Hayashi T. et al. (2018) Nature Communications 9(1): 619); RNAtag-Seq (see e.g., Shishkin A. A. et al. (2015) Nat Methods 12: 323-325); Safe-SeqS (see e.g., Kinde I. et al. (2011) Proc Natl Acad Sci USA 108: 9530-5); scABA-seq (see e.g., Mooijman D. et al. (2016) Nature Biotechnology 34: 852); scATAC-seq (see e.g., Buenrostro J. D. et al. (2015) Nature 523: 486-490 (Microfluidics)); scATAC-Seq (see e.g., Cusanovich D. A. et al. (2015) Science 348: 910-4 (Cell Index)); scChip-seq (see e.g., Rotem A. et al. (2015) Nat Biotechnol 33: 1165-72); scCool-seq (see e.g., Li L. et al. (2018) Nature Cell Biology 20(7): 847-858); sciHi-C (see e.g., Ramani V. et al. (2017) Nature Methods 14: 263); sci-CAR (see e.g., Cao J. et al. (2018) Science 361(6409): 1380); sci-DNA-seq (see e.g., Rosenberg A. B. et al. (2018) Science 360: 176-182); sci-MET (see e.g., Mulqueen R. M. et al. (2018) Nature Biotechnology 36: 428); sci-RNA-seq (see e.g., Cao J. et al. (2017) Science 357(6352): 661); SCMDA (see e.g., Dong X. et al. (2017) Nature Methods 14: 491); scM&T-seq (see e.g., Angermueller C. et al. (2016) Nature Methods 13: 229); scNMT-seq (see e.g., Clark S. J. et al. (2018) Nature Communications 9(1): 781 scRC-Seq Upton K. R. et al. (2015) Cell 161: 228-39); scRNA-seq (see e.g., Tang F. et al. (2009) Nat Methods 6: 377-82); SCRB-Seq Soumillon M. et al. (2014) bioRxiv: 003236); scTHS-seq (see e.g., Lake B. B. et al. (2018) Nature Biotechnology 36(1): 70-80); scTrio-seq (see e.g., Hou Y. et al. (2016) Cell Res 26: 304-19); scTrio-seq2 (see e.g., Bian S. et al. (2018) Science 362(6418): 1060); Seq-Well (see e.g., Gierahn T. M., et al. (2017). Nat Methods 14(4): 395-398); SIDR (see e.g., Han K. Y. et al. (2018) Genome Research 28(1): 75-87); SINC-seq (see e.g., Abdelmoez M. N. et al. (2018) Genome Biology 19(1): 66); Smart-Seq (see e.g., Ramskold D. et al. (2012) Nat Biotechnol 30: 777-782); Smart-seq2 (see e.g., Picelli S. et al. (2013) Nat Methods 10: 1096-1098v); SMDB (see e.g., Lan F. et al. (2016) Nat Commun 7: 11784); smMIP (see e.g., Hiatt J. B. et al. (2013) Genome Res 23: 843-854); snDrop-seq (see e.g., Lake B. B. et al. (2018) Nature Biotechnology 36(1): 70-80); SNES (see e.g., Leung M. L. et al. (2015) Genome Biol 16: 55); snmC-Seq (see e.g., Luo C. et al. (2017) Science 357(6351): 600); snRNA-seq (see e.g., Grindberg R. V. et al. (2013) Proc Natl Acad Sci USA 110: 19802-7); SPLiT-seq (see e.g., Rosenberg A. B. et al. (2018) Science 360(6385): 176); STRT (see e.g., Islam S. et al. (2011) Genome Res 21: 1160-1167); SUPeR-seq (see e.g., Fan X. et al. (2015) Genome Biol 16: 148); TCR Chain Pairing (see e.g., Turchaninova M. A. et al. (2013) Eur J Immunol 43: 507-2515); TCR-LA-MC-PCR (see e.g., Ruggiero E. et al. (2015) Nat Commun 6: 8081); TIVA (see e.g., Lovatt D. et al. (2014) Nat Methods 11: 190-196); TSCS (see e.g., Casasent A. K. et al. (2018) Cell 172(1): 205-217.e212); UMI Method (see e.g., Kivioja T. et al. (2012) Nat Methods 9: 72-74); and viscRNA-seq (see e.g., Zanini F. et al. (2018) Elife 7: e32942).

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International patent application number PCT/US2016/059239, published as WO2017164936 on Sep. 28, 2017; International patent application number PCT/US2018/060860, published as WO/2019/094984 on May 16, 2019; International patent application number PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

### Detecting DNA Methylation

In some cases, the DNA methylation may be detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray. Microarrays, and massively parallel sequencing, have enabled the interrogation of cytosine methylation on a genome-wide scale (Zilberman D, Henikoff S. 2007. Genome-wide analysis of DNA methylation patterns. Development 134(22): 3959-3965.). Genome wide methods have been described previously (Deng, et al. 2009. Targeted bisulfite sequencing reveals changes in DNA methylation associated with nuclear reprogramming. Nat Biotechnol 27(4): 353-360; Meissner, et al. 2005. Reduced representation bisulfite sequencing for comparative high-resolution DNA methylation analysis. Nucleic Acids Res 33(18): 5868-5877; Down, et al. 2008. A Bayesian deconvolution strategy for immunoprecipitation-based DNA methylome analysis. Nat Biotechnol 26(7): 779-785; Gu et al. 2011. Preparation of reduced representation bisulfite sequencing libraries for genome-scale DNA methylation profiling. Nat Protoc 6(4): 468-481).

In some embodiments, DNA methylation may be detected by whole genome bisulfite sequencing (WGBS) (Cokus, et al. 2008. Shotgun bisulphite sequencing of the Arabidopsis genome reveals DNA methylation patterning. Nature 452(7184): 215-219; Lister, et al. 2009. Human DNA methylomes at base resolution show widespread epigenomic differences. Nature 462(7271): 315-322; Harris, et al. 2010. Comparison of sequencing-based methods to profile DNA methylation and identification of monoallelic epigenetic modifications. Nat Biotechnol 28(10): 1097-1105).

In certain cases, DNA methylation may be detected methylation-specific PCR, whole genome bisulfite sequence, the HELP assay and other methods using methylation-sensitive restriction endonucleases, ChiP-on-chip assays, restriction landmark genomic scanning, COBRA, Ms-SNuPE, methylated DNA immunoprecipitation (MeDip), pyrosequencing of bisulfite treated DNA, molecular break light assay for DNA adenine methyltransferase activity, methyl sensitive Southern blotting, methylCpG binding proteins, mass spectrometry, HPLC, and reduced representation bisulfite sequencing. In some embodiments, the DNA methylation is detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray.

A methylation profile can be determined from the methods disclosed herein. In embodiments, the determining the methylation profile comprises generating a genome-wide methylation profile of the cells. Neighborhood methylation profile analysis may be performed by analyzing the loci that any given locus was in contact with to. Such analysis may be used to evaluate can how the chromatin neighborhood affected the methylation state of the DNA of that locus. Aggregate methylation profile may also be performed to sum the methylation profile at a large number of positions and to reveal subtle effects in WGBS data. In some examples, aggregate methylation analysis may be performed by plotting DNA methylation in vicinity of selected sequences (e.g., motifs) and compare it to nucleosome occupancy data (e.g., from MNase-Seq). Methylation profile may comprise unmethylation, methylation and co-methylation at each end of the end-joined nucleic acid fragments.

### Methods of Diagnosing/Prognosing Disease

The methods of multi-omic analysis described herein can be used to diagnose, prognose, and/or monitor a disease or condition in a subject. In some embodiments, the subject is a human. In some embodiments, the subject is a non-human primate. In some embodiments, the subject is an animal. Exemplary animals include, but are not limited to, animals such as fish, amphibians, reptiles, mammals, and birds. The animals may be farm and agriculture animals, or pets. Examples of farm and agriculture animals include horses, goats, sheep, swine, cattle, llamas, alpacas, and birds, e.g., chickens, turkeys, ducks, and geese. The animals may be a non-human primate, e.g., baboons, capuchin monkeys, chimpanzees, lemurs, macaques, marmosets, tamarins, spider monkeys, squirrel monkeys, and vervet monkeys. Examples of pets include dogs, cats horses, wolfs, rabbits, ferrets, gerbils, hamsters, chinchillas, fancy rats, guinea pigs, canaries, parakeets, and parrots.

In some embodiments, the disease is a cancer. In some embodiments, the disease is a non-cancerous disease or disorder. Exemplary non-cancerous diseases include, but are not limited to, autoimmune diseases, allergies and asthma, intestinal diseases and disorders, heart disease and disorders, lung diseases and disorders, sinus diseases and disorders, kidney diseases and disorders, infectious diseases, liver diseases, central and peripheral nervous system diseases and disorders, inflammatory diseases and disorders, pancreatic diseases and disorders, brain diseases and disorders, muscle diseases and disorders, bone diseases and disorders, connective tissue diseases and disorders, metabolic diseases and disorders, skin diseases and disorders, eye diseases and disorders, ear diseases and disorders, nose diseases and disorders, dental diseases and disorders, stomach diseases and disorders, bladder diseases and disorders, prostate diseases and disorders, urinary system diseases and disorders, vaginal, ovarian, and uterine diseases and disorders, testis diseases and disorders, breast diseases and disorders, esophagus diseases and disorders, vascular diseases and disorders, blood disease and disorders, pulmonary diseases and disorders, cerebrovascular diseases and disorders, cardiovascular diseases and disorders, and infections caused by a microorganism.

In some embodiments, a method of diagnosing, monitoring, or prognosing a condition or disease in a subject, comprising: characterizing a feature of one or more individual cells and/or nuclei in the subject or in a sample therefrom at one or more time points using a multi-omic method as described elsewhere herein; and providing a diagnosis, prognosis, or condition or disease status based on one or more features. In some embodiments, the feature(s) are a cellular RNA expression profile; a surface protein expression profile; an epigenetic feature of a genomic DNA region in the cell; or a combination thereof.

In some embodiments, the subject is a plant. In some embodiments, the disease is a plant disease or disorder. In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants. The compositions, systems, and methods may be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g those belonging to the orders Pinales, Ginkgoales, Cycadales, Araucariales, Cupressales and Gnetales.

The compositions, systems, and methods herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

The compositions, systems, and methods may be used over a broad range of plants, such as for example, include those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. The compositions, systems, and methods can be used over a broad range of “algae” or “algae cells.” Examples of algae include eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). Examples of algae species include those of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

Exemplary plant diseases that can be detected and or monitored by the methods described herein include, but are not limited to, those exemplified below:

Rice diseases: blast (Magnaporthe oryzae), helminthosporium leaf spot (Cochliobolus miyabeanus) and bakanae disease (Gibberella fujikuroi);

Diseases of barley, wheat, oats and rye: powdery mildew (Erysiphe graminis), Fusarium head blight (Fusarium graminearum, F. avenaceum, F. culmorum, F. asiaticum, Microdochium nivale), rust (Puccinia striiformis, P. graminis, P. recondita, P. hordei), snow blight (Typhula sp., Micronectriella nivalis), loose smut (Ustilago tritici, U. nuda), bunt (Tilletia caries), eyespot (Pseudocercosporella herpotrichoides) scald (Rhynchosporium secalis), leaf blotch (Septoria tritici), glume blotch (Leptosphaeria nodorum) and net blotch (Pyrenophora teres Drechsler);

Citrus diseases: melanose (Diaporthe citri) and scab (Elsinoe fawcetti);

Apple diseases: blossom blight (Monilinia mali) canker (Valsa ceratosperma), powdery mildew (Podosphaera leucotricha), Alternaria leaf spot (Alternaria alternata apple pathotype) scab (Venturia inaequalis) and bitter rot (Colletotrichum acutatum);

Pear diseases: scab (Venturia nashicola, V. pirina), black spot (Alternaria alternata Japanese pear pathotype) and rust (Gymnosporangium haraeanum);

Peach diseases: brown rot (Monilinia fructicola), scab (Cladosporium carpophilum) and Phomopsis rot (Phomopsis sp.);

Grapes diseases: anthracnose (Elsinoe ampelina), ripe rot (Glomerella cingulata), powdery mildew (Uncinula necator), rust (Phakopsora ampelopsidis), black rot (Guignardia bidwellii) and gray mold (Botrytis cinerea);

Diseases of Japanese persimmon: anthracnose (Gloeosporium kaki) and leaf spot (Cercospora kaki, Mycosphaerella nawae);

Diseases of gourd family: anthracnose (Colletotrichum lagenarium), powdery mildew (Sphaerotheca fuliginea), gummy stem blight (Mycosphaerella melonis) and Fusarium wilt (Fusarium oxysporum);

Tomato diseases: early blight (Alternaria solani) and leaf mold (Cladosporium flavum);

Egg plant disease: brown spot (Phomopsis vexans) and powdery mildew (Erysiphe cichoracearum);

Diseases of Cruciferous Vegetables: Alternaria leaf spot (Alternaria japonica) and white spot (Cercosporella brassicae);

Rapeseed diseases: Sclerotinia rot (Sclerotinia sclerotiorum), black spot (Alternaria brassicae), powdery mildew (Erysiphe cichoracearum), blackleg (Leptosphaeria maculans);

Welsh onion diseases: rust (Puccinia allii);

Soybean diseases: purple seed stain (Cercospora kikuchii), sphaceloma scad (Elsinoe glycines), pod and stem blight (Diaporthe phaseolorum var. sojae) and rust (Phakopsora pachyrhizi);

Adzuki-bean diseases: gray mold (Botrytis cinerea), sclerotinia rot (Sclerotinia sclerotiorum);

Kidney bean diseases: gray mold (Botrytis cinerea), Sclerotinia rot (Sclerotinia sclerotiorum), anthracnose (Colletotrichum lindemthianum);

Peanut diseases: leaf spot (Cercospora personate) brown leaf spot (Cercospora arachidicola) and southern blight (Sclerotium rolfsii);

Garden pea diseases: powdery mildew (Erysiphe pisi);

Strawberry diseases: powdery mildew (Sphaerotheca humuli);

Tea diseases: net blister blight (Exobasidium reticulatum), white scab (Elsinoe leucospila) gray blight (Pestalotiopsis sp.) and anthracnose (Colletotrichum theae-sinensis);

Cotton diseases: Fusarium wilt (Fusarium oxysporum), damping-off (Rhizoctonia solani);

Tobacco diseases: brown spot (Alternaria longipes), powdery mildew (Erysiphe cichoracearum) and anthracnose (Colletotrichum tabacum);

Sugar beet diseases: cercospora leaf spot (Cercospora beticola), leaf blight (Thanatephorus cucumeris) and root rot (Thanatephorus cucumeris);

Rose diseases: black spot (Diplocarpon rosae) and powdery mildew (Sphaerotheca pannosa);

Chrysanthemum diseases: leaf blight (Septoria chrysanthemi-indici) and white rust (Puccinia horiana);

Various plants diseases: gray mold (Botrytis cinerea), Sclerotinia rot (Sclerotinia sclerotiorum),

Japanese radish Disease: Alternaria leaf spot (Alternaria brassicicola);

Turfgrass diseases: dollar spot (Sclerotinia homeocarpa), brown patch and large patch (Rhizoctonia solani); and

Banana diseases: Sigatoka disease (Mycosphaerella fijiensis, Myosphaerella musicola, Pseudocercospora musae)

### Samples

The nucleic acids may be obtained or derived from a sample. A sample, such as a biological sample, may include biological materials (such as nucleic acid and proteins, for example double-stranded nucleic acid binding proteins) obtained from an organism or a part thereof, such as a plant, animal, bacteria, and the like. In particular embodiments, the sample is obtained from an animal subject, such as a human subject. A biological sample may be any solid or fluid sample obtained from, excreted by or secreted by any living organism, including without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood (or fraction(s) or component(s) thereof), plasma, serum, urine, bile, ascites, saliva, cerebrospinal fluid, aqueous or vitreous humor, or any bodily secretion (e.g., mucus, sputum, cervical smear specimens, marrow, feces, sweat, condensed breath, and the like), a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A sample can also be a sample obtained from any organ or tissue (including a biopsy or autopsy specimen, such as a tumor biopsy) or can include a cell (whether a primary cell or cultured cell) or medium conditioned by any cell, tissue or organ. The samples may be fresh, frozen, preserved in fixative (e.g., alcohol, formaldehyde, paraffin, or PreServeCyte™) or diluted in a buffer. Examples of the samples also include, leaves, stems, roots, seeds, petals, pollen, spore, mushroom caps, and sap.

### Methods of Monitoring/Determining an Environmental Condition or State

The methods of multi-omic analysis described herein can be used to determine an environmental condition or state, such as to detect the presence of organisms and/or cells or state of organisms and/or cells within an environment, which can therefore provide information on the state or condition of the environment. In some embodiments, the method can include characterizing a feature of one or more individual cells and/or nuclei in an environmental sample at one or more time points using a multi-omic analysis method as described elsewhere herein; and providing an environmental condition, status, or state based on the feature. Environmental samples can be obtained from a ground water source, earth, surfaces of objects in the environment, air, soil, rain, snow, clouds, ocean, lakes, ponds, streams, rivers, and the like.

### Methods of Screening

In some embodiments, methods of multi-omic analysis described herein can be used to screen for candidate agents or environmental conditions that promote a specific multi-omic expression signature in a cell or nucleus. In some embodiments, such a method includes exposing a cell or cell population to one or more candidate agents and/or environmental conditions and characterizing a feature of one or more individual cells and/or nuclei exposed to the candidate agent and/or environmental condition at one or more time points using a multi-omic method described herein, and selecting agents and/or environmental conditions that result in one or more desired features in the cell and/or nucleus. In some embodiments, the desired feature(s) are a desired cellular RNA expression profile; a desired surface protein expression profile; a desired epigenetic feature of a genomic DNA region in the cell; or a combination thereof.

Further embodiments are illustrated in the following Examples which are given for illustrative purposes only and are not intended to limit the scope of the invention.

### EXAMPLES

### Example 1—Single-Cell Multimodal Profiling of Proteins and Chromatin Accessibility Using PHAGE-ATAC

Multi-modal measurements of single cell profiles are a powerful tool for characterizing cell states and regulatory mechanisms. While current methods allow profiling of RNA along with either chromatin or protein levels, connecting chromatin state to protein levels remains a barrier. This Example demonstrates PHAGE-ATAC, a method that uses engineered camelid single-domain antibody (‘nanobody’)-displaying phages for simultaneous single-cell measurement of surface proteins, chromatin accessibility profiles, and mtDNA-based clonal tracing through a massively parallel droplet-based assay of single-cell transposase-accessible chromatin with sequencing (ATAC-seq). This Example demonstrates PHAGE-ATAC for multimodal analysis in primary human immune cells and for sample multiplexing. Finally, this Example demonstrates construction of a synthetic high-complexity phage library for selection of novel antigen-specific nanobodies that bind cells of particular molecular profiles, opening a new avenue for protein detection, cell characterization and screening with single-cell genomics. The methods demonstrated by the Examples and elsewhere herein can overcome limitations burdening current multi-omic approaches such as cellular indexing of transcriptomes and epitopes by sequencing (CITE-seq), such as limited by availability of antigen-specific antibodies and costs, and also addresses the lack of technologies for the combined high-throughput measurement of the epigenome and proteome.

Massively-parallel single-cell profiling has become an invaluable tool for the characterization of cells by their transcriptome or epigenome, deciphering gene regulation mechanisms, and dissecting cellular ecosystems in complex tissues (Klein et al., 2015; Lareau et al., 2019; Macosko et al., 2015; Satpathy et al., 2019). In particular, recent advances have highlighted the power of multimodal single-cell assays (Ma et al., 2020), such as cellular indexing of transcriptomes and epitopes by sequencing (CITE-seq), that profile both transcriptome and proteins by DNA-barcoded antibodies (Mimitou et al., 2019; Peterson et al., 2017; Stoeckius et al., 2017).

Although the vast combinatorial space of oligonucleotide barcodes theoretically allows parallel quantification of an unrestricted number of epitopes, in practice, however, we are limited by the availability of antigen-specific antibodies. Moreover, each antibody must be separately conjugated with a unique oligonucleotide (oligo)-barcode, which currently does not allow a scalable and pooled construction of barcoded antibody libraries. Finally, technologies for the combined high-throughput measurement of the epigenome and proteome have not been described.

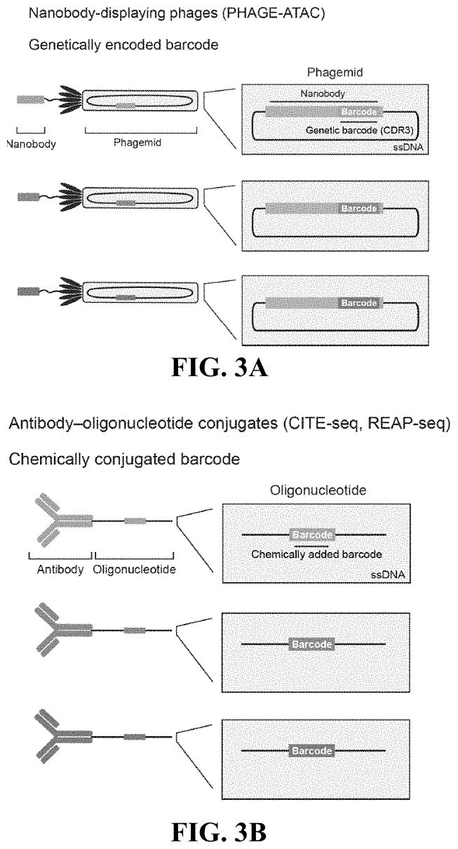

To overcome at least these limitations, PHAGE-ATAC was developed (see e.g., FIGS. 1A-1C and 3A-3B). PHAGE-ATAC is a multimodal single-cell approach for phage-based multiplex protein measurements and chromatin accessibility profiling using the droplet-based scATAC-seq (10× Genomics scATAC (Satpathy et al., 2019)). PHAGE-ATAC enables sensitive quantification of epigenome and proteins, captures mtDNA that can be used as a native clonal tracer (Lareau et al., 2020; Ludwig et al., 2019), introduces phages as renewable and cost-effective reagents for high-throughput single-cell epitope profiling, and leverages phage libraries for the selection of antigen-specific antibodies (Hoogenboom, 2005; Smith, 1985), altogether providing a novel platform that greatly expands the scope of the single-cell profiling toolbox.

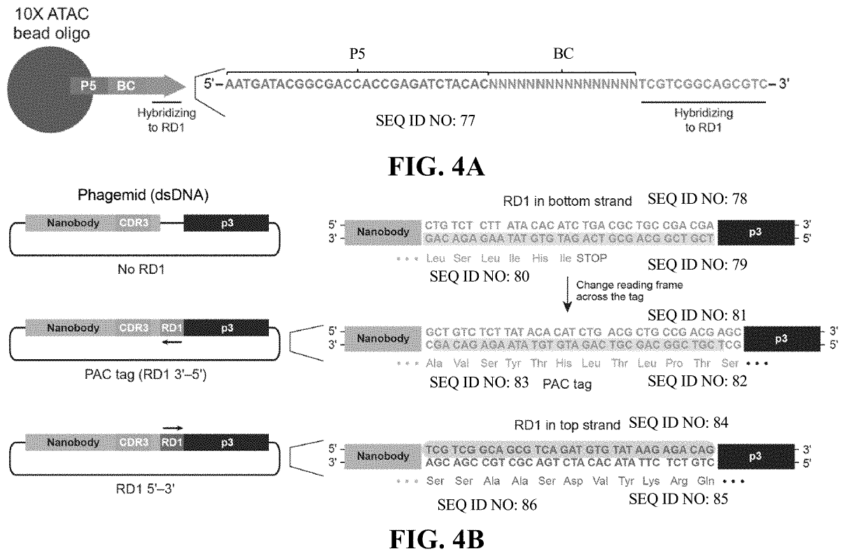

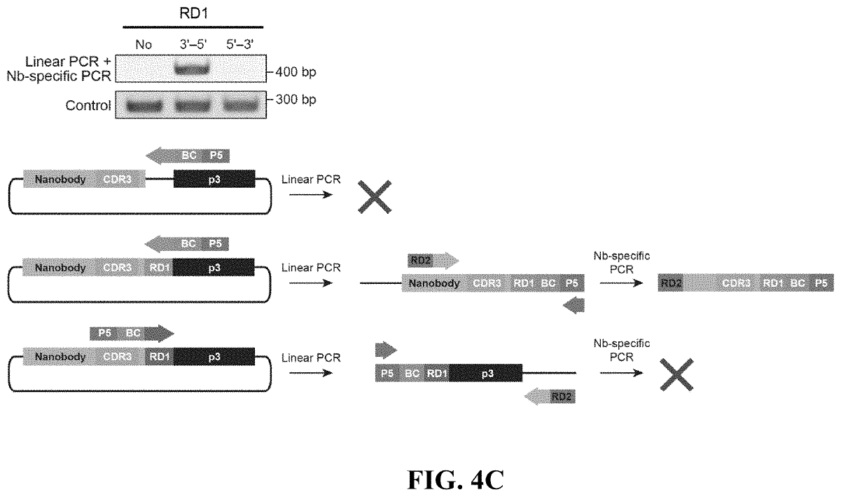

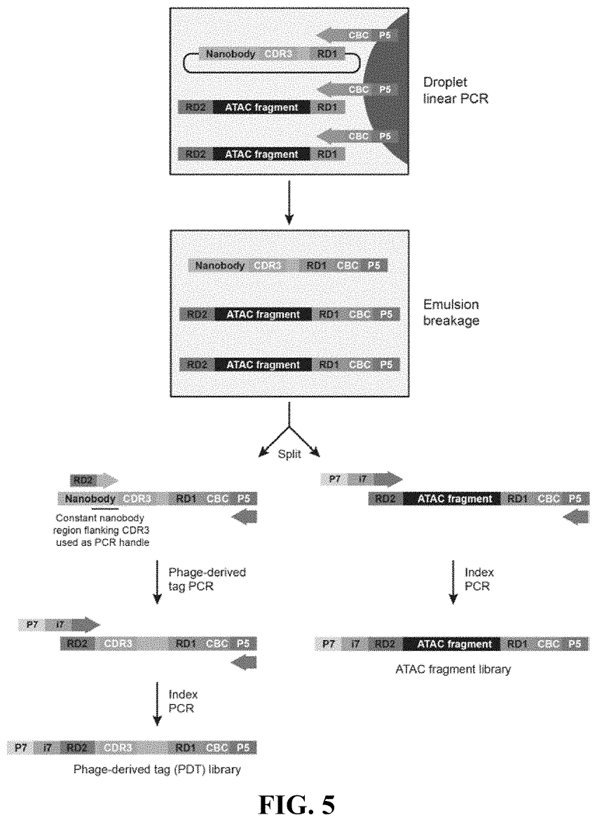

Protein quantification in PHAGE-ATAC is based on epitope recognition by nanobody (Ingram et al., 2018) (Nb)-displaying phages (FIG. 1A, FIGS. 3A-3B), in contrast to recognition by oligonucleotide-conjugated antibodies in CITE-seq and related methods (Peterson et al., 2017; Stoeckius et al., 2017), or fluorescently labeled antibodies in other techniques (Katzenelenbogen et al., 2020; Paul et al., 2015). The hypervariable complementarity-determining region 3 (CDR3) within each Nb-encoding phagemid acts as a unique genetic barcode (Pollock et al., 2018) that is identified by sequencing in PHAGE-ATAC and serves as a proxy for antigen detection and quantification (FIG. 1A, FIGS. 3A-3B). To allow phage-based epitope quantification alongside accessible chromatin using droplet-based scATAC-seq, we engineered an M13 phagemid for the in-frame expression of (1) an epitope-binding Nb, (2) a PHAGE-ATAC tag (PAC-tag) containing the Illumina Read 1 sequence (RD1) and (3) the phage coat protein p3 for surface display (FIGS. 1A-1B). This enables phage Nb (pNb)-based recognition of cell surface antigens, simultaneous droplet-based indexing of phagemids and ATAC fragments, as well as separate generation of phage-derived tag (PDT) and ATAC sequencing libraries (FIG. 1C, FIGS. 4A-4C, and FIG. 5).

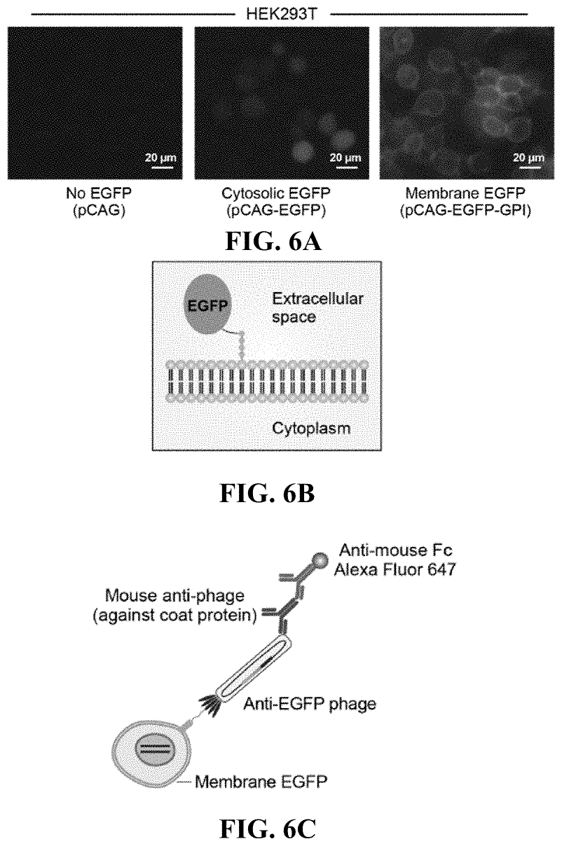

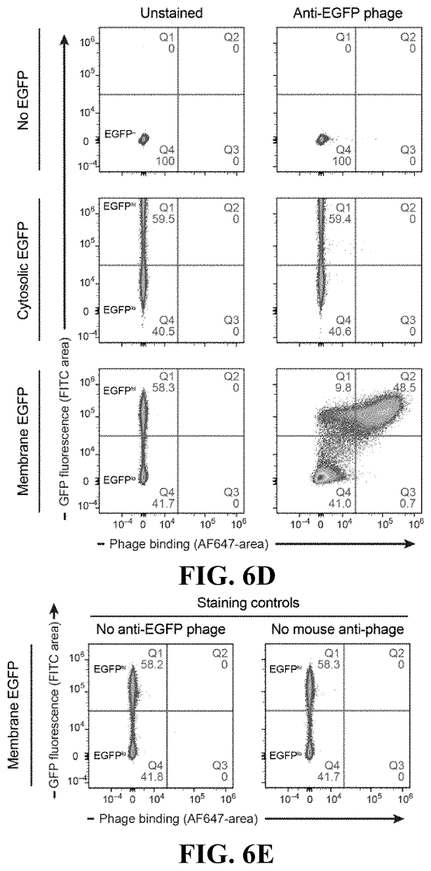

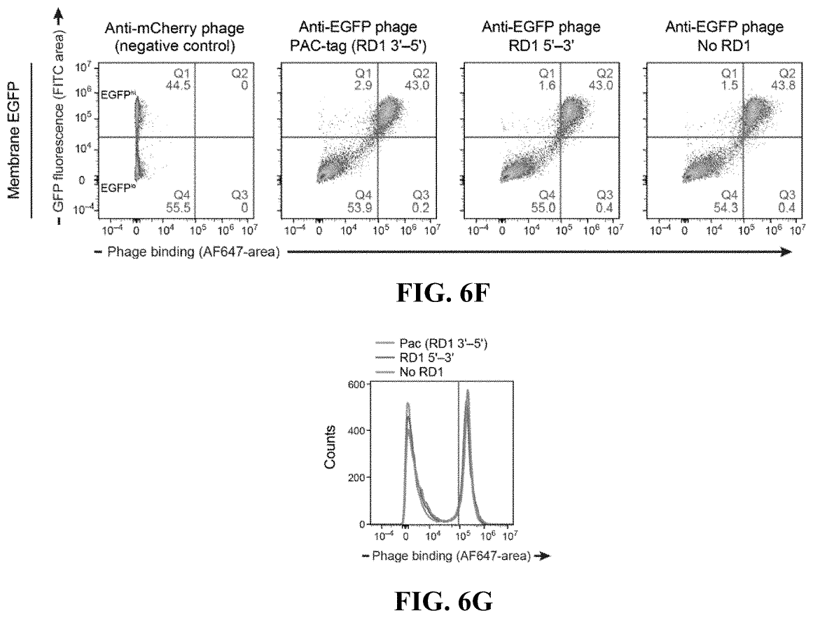

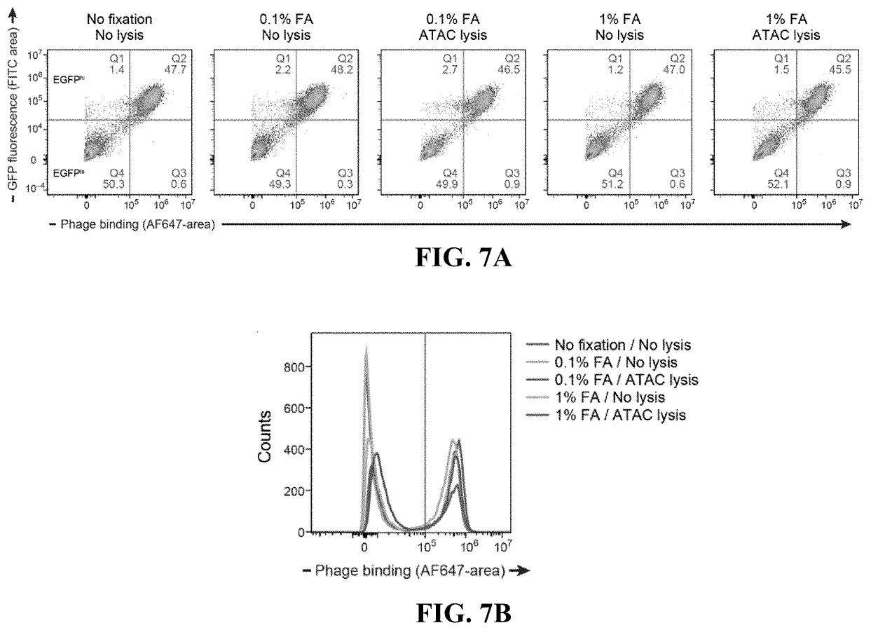

It was first confirmed that the PHAGE-ATAC modified phagemid workflow allows successful and specific pNb antigen recognition and pNb-based cell staining during scATAC cell lysis. As a first proof-of-concept, HEK293T cells expressing surface-exposed glycosylphosphatidyl-inositol (GPI)-anchored EGFP (EGFP-GPI) that are specifically recognized by an anti-EGFP pNb were used (Rothbauer et al., 2006) (FIG. 6A-6E). Importantly, introducing the PAC-tag did not impair Nb display and antigen recognition (FIGS. 6F and 6G). Moreover, fixation retained pNb-based cell staining after the scATAC lysis step, with a standard scATAC-seq buffer (FIG. 7A-7B and Methods herein)

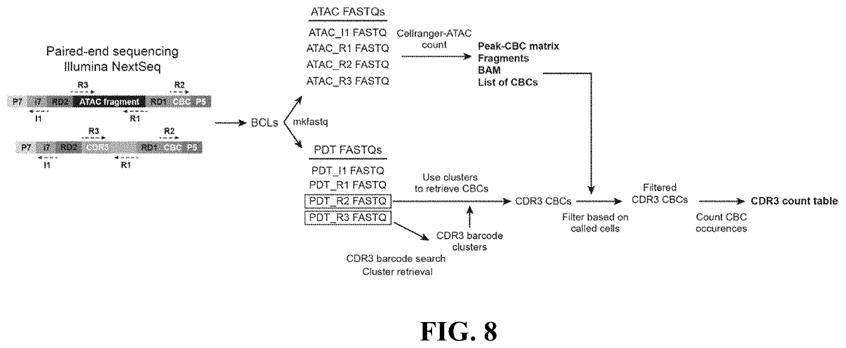

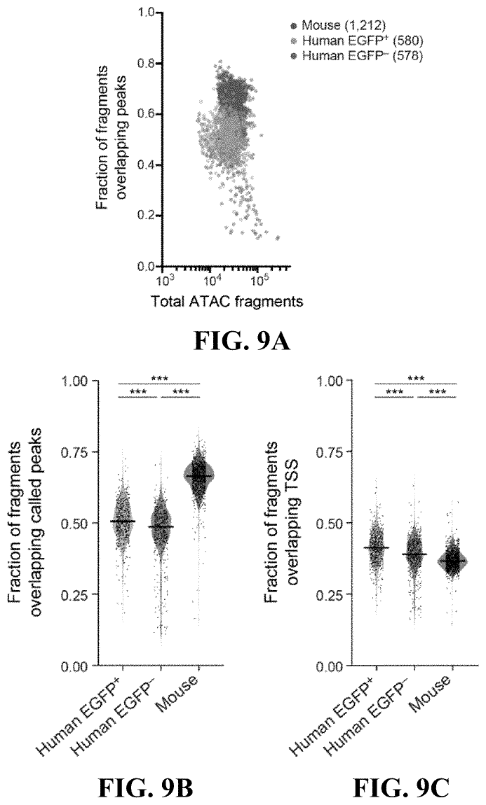

To benchmark PHAGE-ATAC for single cell profiling, we performed a ‘species-mixing’ experiment, in which we pooled mouse (NIH3T3), human EGFP⁻ (HEK293T) and human EGFP (HEK293T-EGFP-GPI) cells at a 2:1:1 ratio, followed by anti-EGFP pNb staining, library generation and analysis using a custom computational workflow (FIG. 1D and FIG. 8, and Methods herein). After filtering, 1,212 mouse and 1,158 human cell barcodes were recovered (FIG. 1E), with good library complexity, enrichment of fragments in peaks, and enrichment in transcription start sites (FIGS. 9A-9C), all comparable to gold-standard published reference data without additional protein detection (Lareau et al., 2020; Satpathy et al., 2019). Analysis of EGFP PDT counts confirmed the presence of EGFP and EGFP⁻ cells (FIGS. 1F and 1G) that together with mouse cell barcodes were all recovered at expected input ratios (observed 2.09:1:1, expected 2:1:1), with no substantial differences in scATAC-seq data quality metrics (FIG. 1H and FIGS. 9A-9C). EGFP PDT levels by PHAGE-ATAC (FIGS. 1F-1G) and EGFP fluorescence intensities by standard flow cytometry (FIG. 1I) were highly concordant (FIGS. 1J-1K). Taken together, these results established the use of PDTs for accurate and sensitive epitope quantification in single cells concomitantly with scATAC-seq.

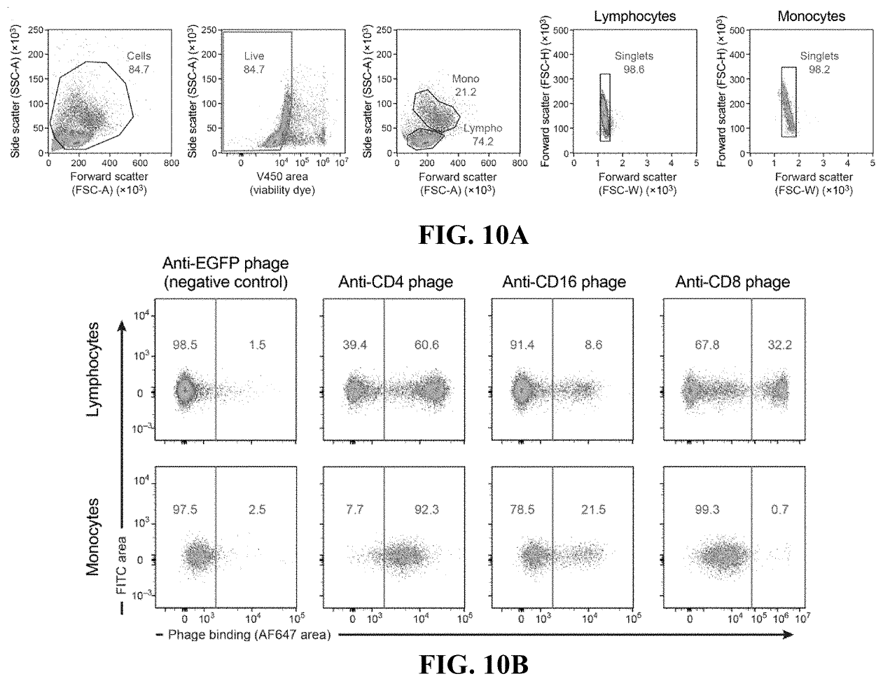

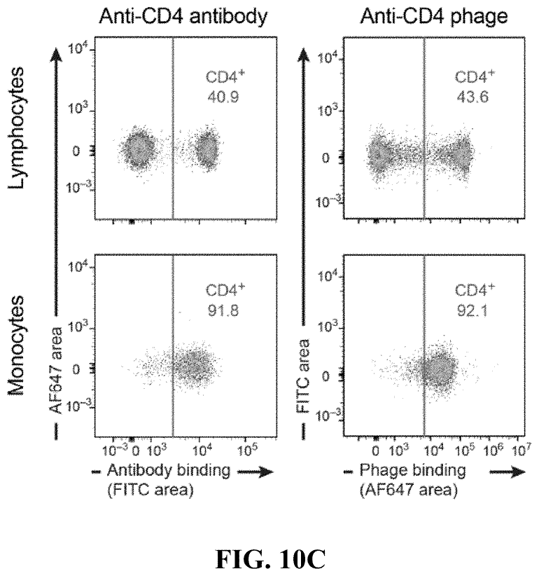

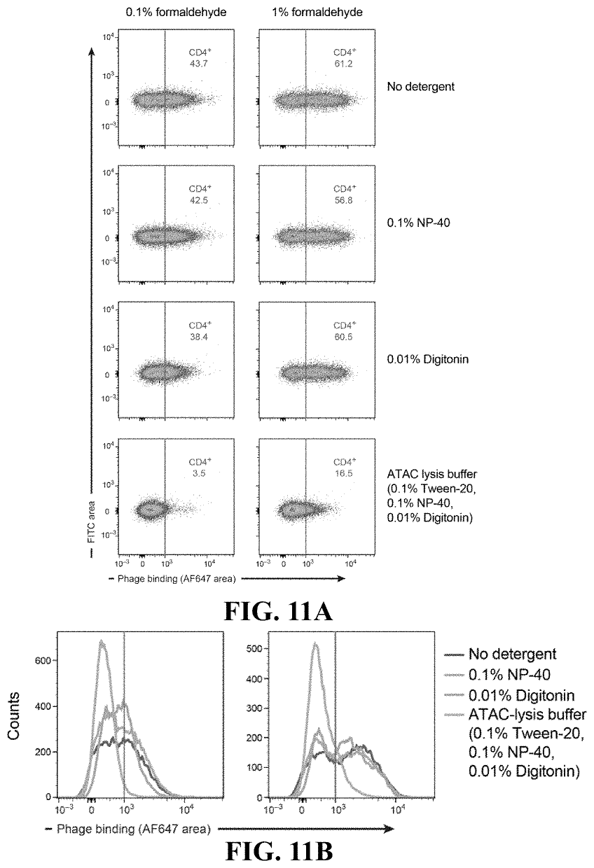

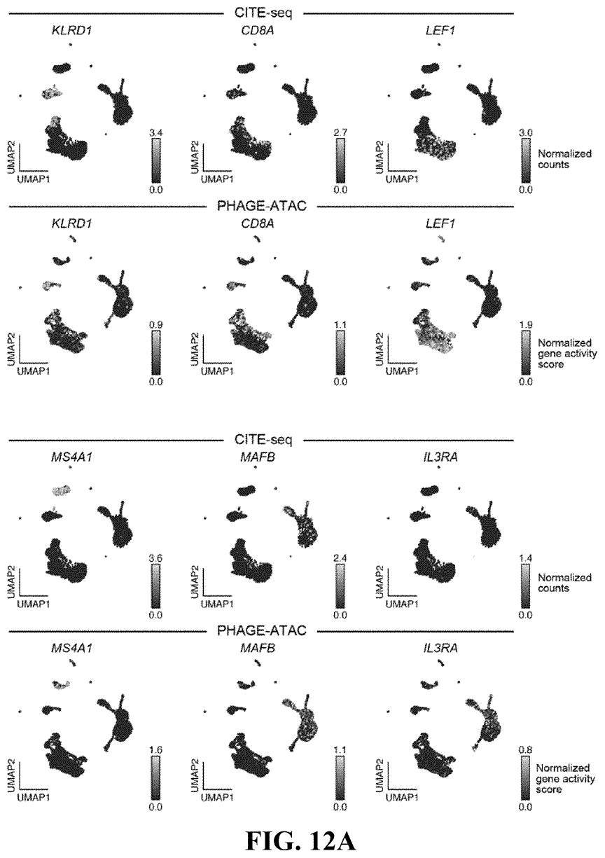

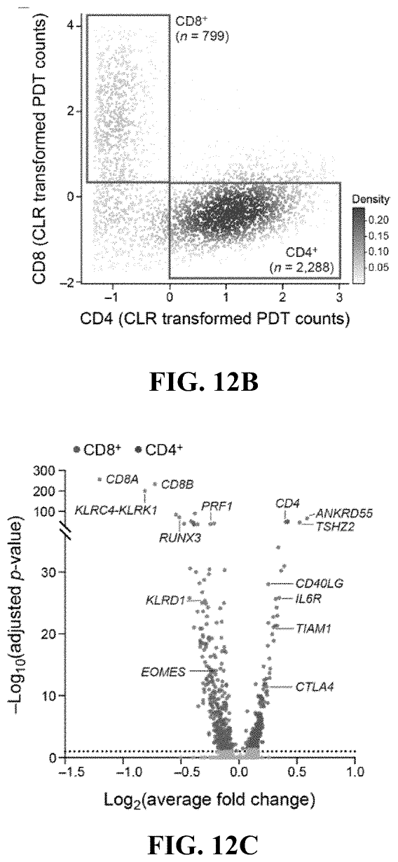

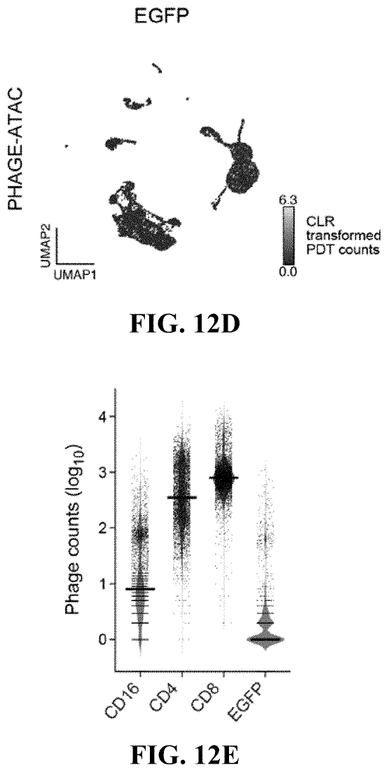

Next, it was demonstrated that PHAGE-ATAC can discern cellular states of primary peripheral blood mononuclear cells (PBMCs) comparably to CITE-seq. For PHAGE-ATAC, well-characterized markers were targeted via a panel of three pNbs targeting CD4, CD8 and CD16 using previously reported high-affinity Nb sequences (Roobrouck et al., 2016; Tavernier et al., 2017), as well as anti-EGFP as a negative control (Methods herein). Flow cytometry of pNb-stained PBMCs and side-by-side comparison between pNb and conventional antibody-stained cells confirmed the antigen-specificity of the produced phages (FIGS. 10A-10C). In addition, the PHAGE-ATAC lysis buffer was further optimized to better preserve phage staining (Lareau et al., 2020) (FIGS. 11A-11B, Methods). Integrative canonical correlation analysis (Butler et al., 2018), clustering and dimensionality reduction of PHAGE-ATAC data of 7,972 high-quality PBMCs and published CITE-seq data of 7,660 PBMCs (Stoeckius et al., 2017) (FIG. 1L, Methods) identified the same set of expected cell states and markers (FIG. 1L and FIG. 12A). The distribution of PDTs and CITE-seq antibody-derived tags (ADTs) across all cell types were highly correlated for each surface marker (FIGS. 1M-1N, Pearson's r=0.69-0.94). To further validate PDT partitioning independently of CITE-seq, we determined differential gene activity scores from the PHAGE-ATAC data alone by comparing scATAC profiles of T cells based on CD4 and CD8 PDT abundances (FIGS. 12B-12C). This identified both CD4 and CD8 loci as top hits and recovered many known bona fide markers of CD4+ and CD8+ T cells (e.g., CD4: CTLA4, CD40LG, ANKRD55; CD8: PRF1, EOMES, RUNX3, FIG. 12C). Finally, EGFP PDTs were only detected at background levels, confirming the high specificity of pNbs (FIGS. 12D-12E). These results illustrate the capacity of PHAGE-ATAC to reliably and specifically detect endogenous cell surface proteins in single cells along with their epigenomic profiles

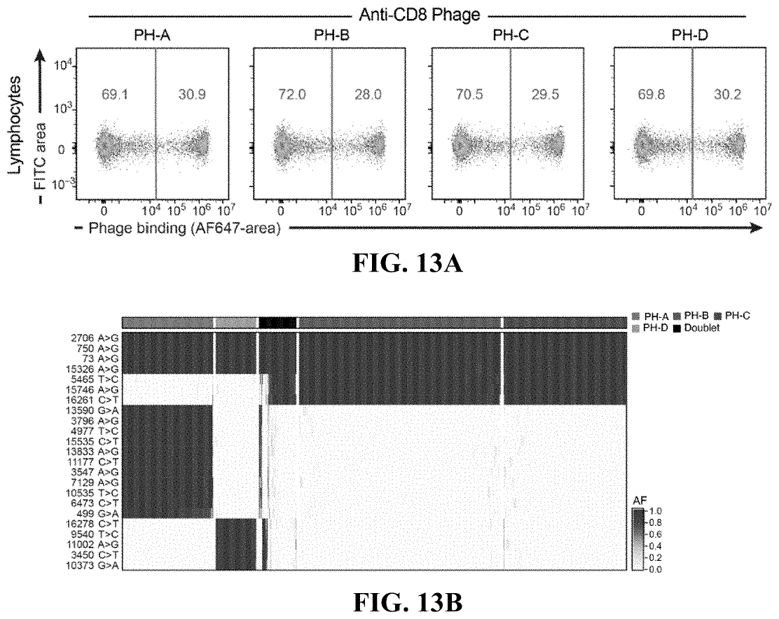

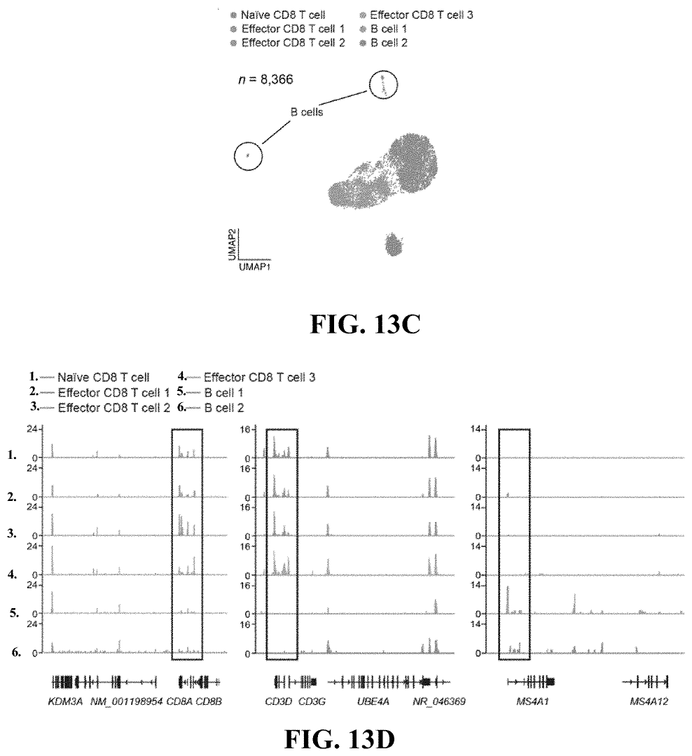

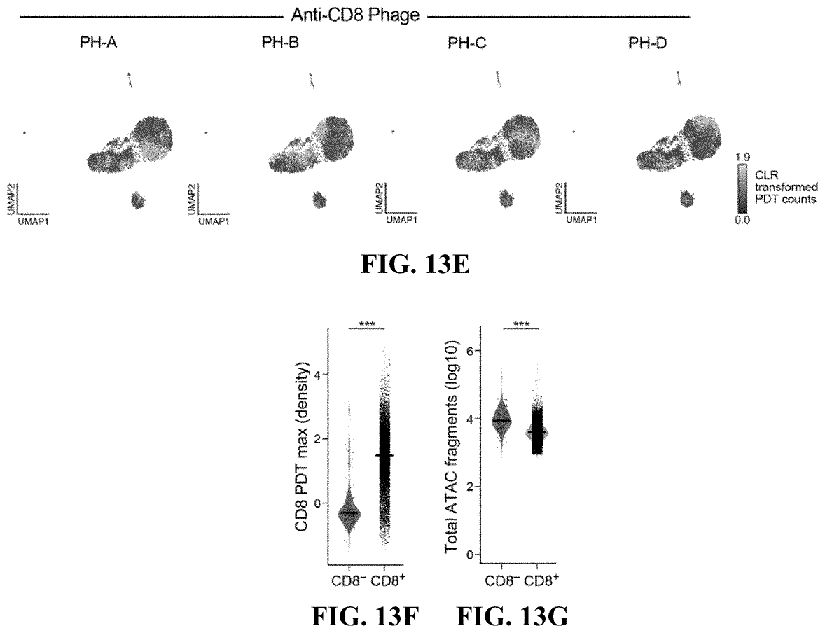

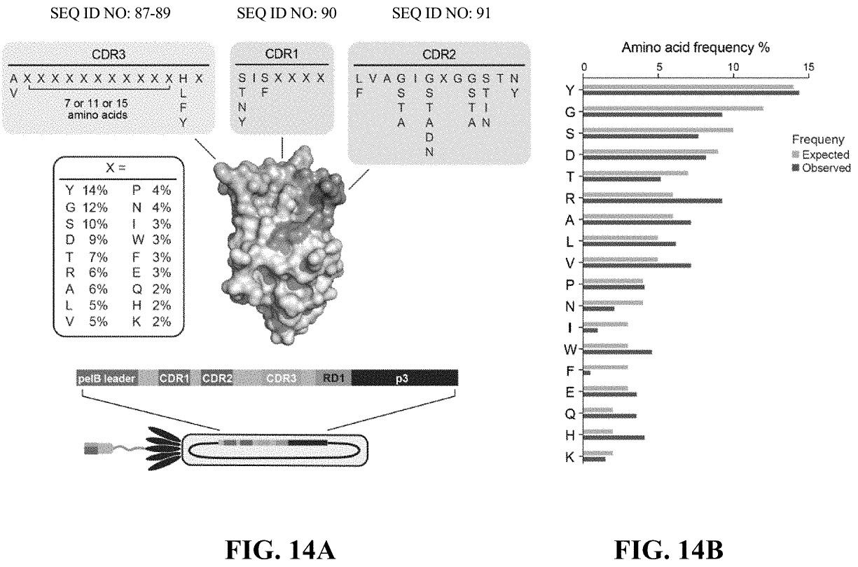

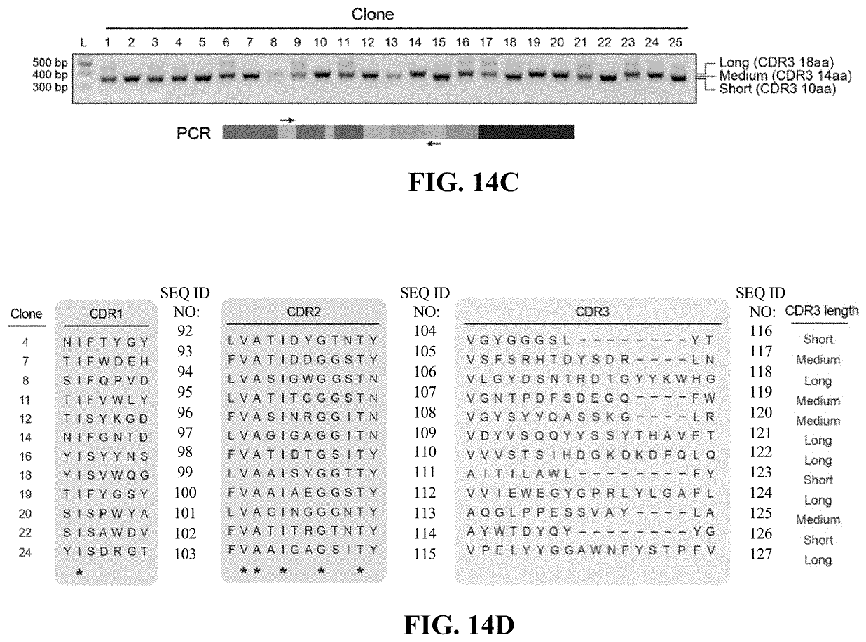

To scale PHAGE-ATAC, a cost-effective alternative for sample multiplexing in scATAC-seq using pNbs for Cell Hashing was introduced. A number of current methods allow ‘overloading’ antibody-tagged cells into droplets to increase single-cell processing throughput and mitigate batch effects (Gehring et al., 2020; Lareau et al., 2019; McGinnis et al., 2019; Stoeckius et al., 2018). To demonstrate hashtags for PHAGE-ATAC, four anti-CD8 hashtag pNbs (henceforth referred to as hashtags) were generated by introducing different silent mutations into the anti-CD8 CDR3 (FIG. 2A, Methods herein), allowing sequencing-based identification of the four hashtags. As expected, the hashtags displayed comparable CD8 recognition within PBMCs (FIG. 13A) To demonstrate phage-based hashing, CD8 T cells from each of four healthy donors were stained with a unique hashtag, pooled them and processed the pool by PHAGE-ATAC, overloading 20,000 cells (FIG. 2A) (vs. about 6000 cells without overloading). These yielded high-quality data for 8,366 cell barcodes, to which Applicant assigned donor and singlet/doublet status from hashtag counts (Methods), identifying the sample of origin for 6,438 singlets and 703 doublets (observed doublet rate 8.4% compared to 10% expected) (FIG. 2B). As expected, barcodes assigned to an individual hashtag had higher count distributions for the respective hashtag (FIG. 2C). Singlet and doublet assignments were concordant with a two-dimensional embedding of hashtag count data (FIG. 2D) with the expected higher numbers of chromatin fragments and hashtag counts in doublets (p<2.2×10-16; Mann-Whitney test, (FIGS. 2E-2F). The hashtag-based assignments were also highly concordant with assignments based on computationally derived donor genotypes from accessible chromatin profiles (Heaton et al., 2020) (Methods herein), with a singlet classification accuracy of 99.3% and an overall classification accuracy of 92.9% (FIG. 2G) Interestingly, chromatin accessibility analyses revealed a small set of putative B cells (FIGS. 13B-13C) consistent with the presence of a minor contaminating population after CD8 T cell enrichment. While B cells were classified as hashtag-negative, genotype and hashtag-based classification were highly consistent across CD8 T cell states (FIG. 211 and FIGS. 13D-13F) confirming hashtag antigen specificity.

PHAGE-ATAC also enables the concomitant capture of mitochondrial genotypes via mitochondrial DNA-derived Tn5 fragments (Lareau et al., 2020), providing a third data modality that relates protein and accessible chromatin profiles to cell clones. Mitochondrial genotyping using mgatk (Lareau et al., 2020) was broadly concordant with the hashtag assignments, but showed that two donors (PH-B and PH-C) had indistinguishable mitochondrial haplotypes, whereas each of the other two donors had several distinguishing mitochondrial variants (FIG. 13G). Collectively, these results established the use of hashtag pNbs for sample multiplexing in scATAC-seq, and its ability to capture mtDNA for clonal analysis.

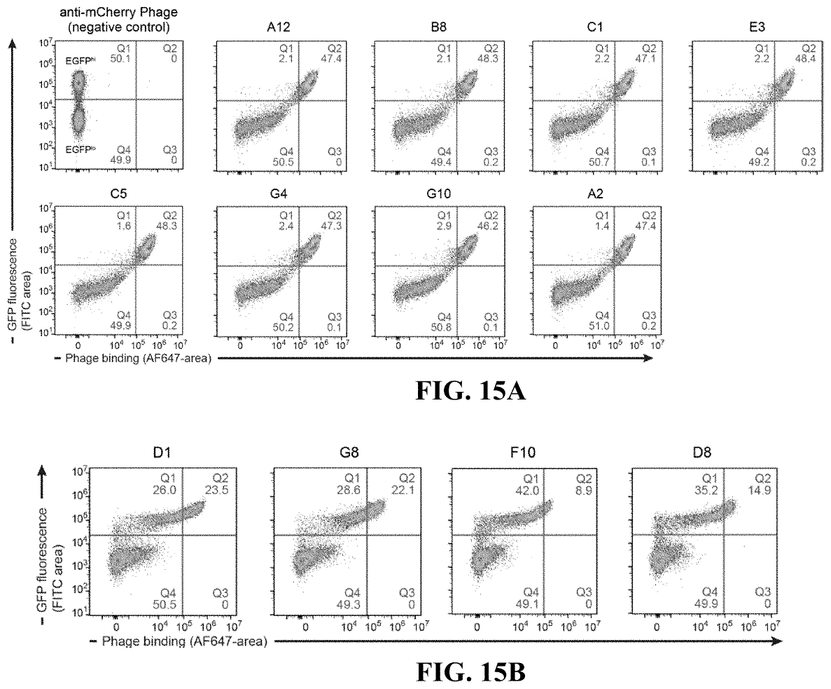

The production of novel high-quality antigen-specific antibodies is laborious, expensive and limited by animal immunization, generating a bottleneck for antibody-based protein profiling. In contrast, recombinant antibody technology based on phage display has allowed fast and cost-effective selection of high-affinity binders (Miersch and Sidhu, 2012). To enable rapid generation of novel antigen-specific pNbs for PHAGE-ATAC, we developed PHAGE-ATAC Nanobody Library (PANL), a synthetic high-complexity (4.96×109) pNb library (Supp. FIG. 12). To demonstrate identification of novel pNbs using PANL, Applicant performed a selection against EGFP-GPI-expressing HEK293T cells, while counter-selecting using parental HEK293T (FIG. 2I). Over three selection rounds, we monitored the enrichment of pNbs by staining EGFP-GPI+ cells, revealing a steady increase of antigen-recognizing pNbs with each additional round (FIG. 2J). Screening of 94 clones after the final (third) selection demonstrated that at least 95% of clones recognized EGFP-GPI+ cells with strong binding (Q2/Q1>1) (FIG. 2K and FIGS. 15A-15B). As clones varied in their ability to bind EGFP-GPI+ cells, Applicant picked 7 clones (5 strong and 2 weak binders) and sequenced their phagemid inserts. Sanger sequencing uncovered the presence of multiple identical clones (A2 and Cl, B8 and E3, FIG. 2L), illustrating selection-driven convergence. Finally, side-by-side comparison of a selected clone (C5) and a reported high-affinity anti-EGFP Nb derived from immunized animals (Rothbauer et al., 2006) indicated similar binding to EGFP-GPI+ cells (FIG. 2M). These results demonstrate the utility of PANL for the rapid selection of pNbs to detect and quantify antigens of interest on cells. They further illustrate PANL's potential for the generation of a new toolbox of barcoded affinity reagents for single cell genomics.

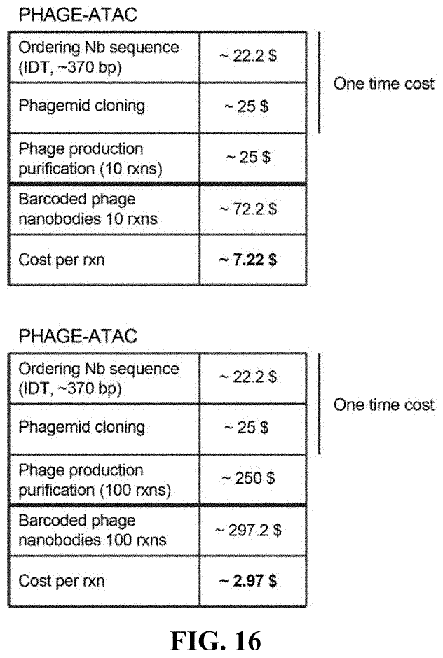

In conclusion, PHAGE-ATAC uses the power of recombinant phage display technology as the basis for single cell profiling of cell surface proteins, chromatin accessibility and mtDNA. This allows users to leverage the renewable nature, low cost and scalability of pooled phage library preparation as well as the compact size and stability of nanobodies (Ingram et al., 2018). PHAGE-ATAC is envisioned as an adaptive tool may be further combined with unique molecular identifiers for phagemid counting and other engineerable scaffolds used in phage display applications (e.g., scFv, Fab) (Gebauer and Skerra, 2009). In the future, we believe this will significantly enhance our ability for the cost-effective (FIG. 16) multimodal single-cell characterization of the proteome, epigenome and likely additional readouts at an unprecedented depth and specificity.

### Example 2—Methods for Example 1

### Oligonucleotides

Oligonucleotide sequences are listed in Table 2. Oligonucleotides were ordered from Integrated DNA Technologies (IDT) unless indicated otherwise.

TABLE 2

| | | SEQ |
|---|---|---|
| | | ID |
| Name | SEQUENCE (5′-3′) | NO: |
| EF05 | ATATATGCTCTTCTAGTATG | 2 |
| | CAGGTTCAACTGGTGGA | |
| EF06 | TATATAGCTCTTCATGCAGA | 3 |
| | GCTCACCGTCACCTGA | |
| EF07 | ATATATGCTCTTCTAGTATG | 4 |
| | GCACAGGTTCAGCTGG | |
| EF08 | TATATAGCTCTTCATGCTGT | 5 |
| | AAACGGGCTGCTAACGG | |
| EF73 | AGCTCTGCAGGAAGAGCTGC | 6 |
| | TGTCTCTTATACACATCTGA | |
| | CGCTGCCGACGAGCTACCCG | |
| | TACGACGTTCCG | |
| EF74 | CGGAACGTCGTACGGGTAGC | 7 |
| | TCGTCGGCAGCGTCAGATGT | |
| | GTATAAGAGACAGCAGCTCT | |
| | TCCTGCAGAGCT | |
| EF75 | AGCTCTGCAGGAAGAGCTTC | 8 |
| | GTCGGCAGCGTCAGATGTGT | |
| | ATAAGAGACAGTACCCGTAC | |
| | GACGTTCCG | |
| EF76 | CGGAACGTCGTACGGGTACT | 9 |
| | GTCTCTTATACACATCTGAC | |
| | GCTGCCGACGAAGCTCTTCC | |
| | TGCAGAGCT | |
| EF77 | GTGTCTGCAGGAAGAGCTGC | 10 |
| | TGTCTCTTATACACATCTGA | |
| | CGCTGCCGACGAGCTACCCG | |
| | TACGACGTTCCG | |
| EF78 | CGGAACGTCGTACGGGTAGC | 11 |
| | TCGTCGGCAGCGTCAGATGT | |
| | GTATAAGAGACAGCAGCTCT | |
| | TCCTGCAGACAC | |
| EF79 | AACAGTCTGAAGCCGGAGGA | 12 |
| | TACCGCGGTGTATTATTGCA | |
| | ATGTCAACGTGGGGTTT | |
| EF80 | AAACCCCACGTTGACATTGC | 13 |
| | AATAATACACCGCGGTATCC | |
| | TCCGGCTTCAGACTGTT | |
| EF17 | GACAACGCCTGTAGCATTCC | 14 |
| EF52 | TCGTCGGCAGCGTCAGATGT | 15 |
| | GTATAAGAGACAGCAGCCTG | |
| | CGCCTGAGCTG | |
| EF53 | GTCTCGTGGGCTCGGAGATG | 16 |
| | TGTATAAGAGACAGCCTGGG | |
| | TGCCCTGGCCCCAATA | |
| EF147 | AATGATACGGCGACCACCGA | 17 |
| | GA | |
| EF91 | GTCTCGTGGGCTCGGAGATG | 18 |
| | TGTATAAGAGACAGgatacc | |
| | gcggtgtattattgc | |
| EF104 | ATATATGCTCTTCTAGTATG | 19 |
| | CAGGTCCAGCTCCAAGA | |
| EF105 | TATATAGCTCTTCATGCGCT | 20 |
| | CGACACCGTTACTTGTG | |
| EF87 | ATATATGCTCTTCTAGTATG | 21 |
| | GAAGTTCAACTTGTAGAGAG | |
| EF88 | TATATAGCTCTTCATGCGCT | 22 |
| | GCTCACGGTGACCTGG | |
| EF89 | TATATAGCTCTTCATGCGCT | 23 |
| | GCTCACTGTTACCTGG | |
| EF156 | CGCGGTGTATTATTGCGCAA | 24 |
| | AGGACGCGGACCTGGTATGG | |
| | TAC | |
| EF157 | GTACCATACCAGGTCCGCGT | 25 |
| | CCTTTGCGCAATAATACACC | |
| | GCG | |
| EF158 | CGCGGTGTATTATTGCGCTA | 26 |
| | AAGACGCGGACCTGGTATGG | |
| | TAC | |
| EF159 | GTACCATACCAGGTCCGCGT | 27 |
| | CTTTAGCGCAATAATACACC | |
| | GCG | |
| EF164 | CGGACAAGGAACACAAGTTA | 28 |
| | CGGTAAGCAGCGCAGGAAGA | |
| | GCTGCT | |
| EFI65 | AGCAGCTCTTCCTGCGCTGC | 29 |
| | TTACCGTAACTTGTGTTCCT | |
| | TGTCCG | |
| EF166 | AACCGGACAAGGAACACAGG | 30 |
| | TCACTGTAAGCAGCGCAGGA | |
| | AGAGCTGCT | |
| EFI67 | AGCAGCTCTTCCTGCGCTGC | 31 |
| | TTACAGTGACCTGTGTTCCT | |
| | TGTCCGGTT | |
| EF64 | CGCGGCGAGCGGCWMTATTT | 32 |
| | YTXXXXATGGGCTGGTATCG | |
| | CCAGG | |
| EF65 | CCGGGCAAAGAACGCGAAYT | 33 |
| | TGTTGCCRSTATTRVTXGGT | |
| | RSTANTACCWATTATGCGGA | |
| | TAGCGTGAAAGGCC | |
| EF66 | CCGCGGTGTATTATTGCGCG | 34 |
| | GYTXXXXXXXYWTXTATTGG | |
| | GGCCAGGGCACC | |
| EF67 | CCGCGGTGTATTATTGCGCG | 35 |
| | GYTXXXXXXXXXXXYWTXTA | |
| | TTGGGGCCAGGGCACC | |
| EF68 | CCGCGGTGTATTATTGCGCG | 36 |
| | GYTXXXXXXXXXXXXXXXYW | |
| | TXTATTGGGGCCAGGGCACC | |
| EF42 | CAGGTGCAGCTGCAGGAAAG | 37 |
| | CGGCGGCGGCCTGGTGCAGG | |
| | CGGGCGGCAG | |
| EF43 | GCCGCTCGCCGCGCAGCTCA | 38 |
| | GGCGCAGGCTGCCGCCCGCC | |
| | TGC | |
| EF44 | TTCGCGTTCTTTGCCCGGCG | 39 |
| | CCTGGCGATACCAGCCCAT | |
| EF45 | GTTTTTCGCGTTATCGCGGC | 40 |
| | TAATGGTAAAGCGGCCTTTC | |
| | ACGCTATCCGCATA | |
| EF46 | AGCCGCGATAACGCGAAAAA | 41 |
| | CACCGTGTATCTGCAGATGA | |
| | ACAGCCTGAAACC | |
| EF47 | CGCGCAATAATACACCGCGG | 42 |
| | TATCTTCCGGTTTCAGGCTG | |
| | TTCATCTGCAGA | |
| EF48 | GCTGCTCACGGTCACCTGGG | 43 |
| | TGCCCTGGCCCCAATA | |
| EF40 | ATATATGCTCTTCTAGTCAG | 44 |
| | GTGCAGCTGCAGGAAAG | |
| EF41 | TATATAGCTCTTCATGCGCT | 45 |
| | GCTCACGGTCACCTGG | |
| EF170 | AATGATACGGCGACCACCGA | 46 |
| | GATCTACACCTCTCTATTCG | |
| | TCGGCAGCGTC | |
| EF57 | GTCTCGTGGGCTCGGAGATG | 47 |
| | TGTATAAGAGACAGAGCTGT | |
| | GCCGCAAGCGGT | |
| EF58 | GTCTCGTGGGCTCGGAGATG | 48 |
| | TGTATAAGAGACAGAGCTGT | |
| | GCAGCAAGCGGT | |

### Cloning of Phagemids for Display of PAC-Tagged Nanobody-p3 Fusions for PHAGE-ATAC

Based on the 10×scATAC bead oligo design (FIG. 4A), it was hypothesized that introduction of an RD1 flanking the Nb CDR3 barcode would enable barcode capture alongside accessible chromatin fragments during droplet-based indexing. To avoid premature termination of nanobody-p3 fusion translation due to the introduction of RD1, the RD1-spanning reading frame was modified, which resulted in the expression of a 12-amino acid PHAGE-ATAC tag (PAC-tag). To generate a phagemid for C-terminal fusion of both PAC-tag and p3, 20 ng pDXinit (Addgene ID: 110101) were subjected to site-directed mutagenesis with primers EF77 and EF78 using PfuUltraII (Agilent) in 50 μl reactions. PCR conditions were 95° C. 3 min; 19 cycles 95° C. 30 sec, 60° C. 1 min, 68° 12 min; final extension 72° C. 14 min. Template DNA was digested for 1.5 h at 37° C. by addition of 1.5 μl DpnI (Fastdigest, Thermo Scientific). PCR reactions were then purified using GeneJet Gel Extraction Kit (Thermo Scientific) and eluted in 45 μl water. 20 μl eluate were transformed into chemically-competent E. coli (NEB Stable Competent) and plated on LB-Ampicillin, yielding pDXinit-PAC. For cloning of nanobody-PAC-p3 fusion-encoding phagemids, nanobody sequences listed in Table 3 were ordered as gBlocks from IDT. 25 ng nanobody gBlocks were first amplified by PCR to introduce SapI restriction sites. Hereby, primers EF87 and EF88 were used for CD4 Nb, primers EF87 and EF89 for CD16 Nb and primers EF104 and EF105 for CD8 Nb. 50 μl PCR reactions using Q5 (NEB) were cycled 98° C. 1 min; 35 cycles 98° C. 15 sec, 60° C. 30 sec, 72° 30 sec; final extension 72° C. 3 min. PCR reactions were loaded on a 1% agarose gel, expected bands were cut and PCR products were extracted using GeneJet Gel Extraction Kit (Thermo Scientific) and eluted in 40 μl water. Cloning was performed using the FX system as described previously (PMID: 21410291). Briefly, each eluted insert was mixed with 50 ng pDXinit-PAC in a molar ratio of 1:5 (vector:insert) in 10 μl reactions and digested with 0.5μl SapI (NEB) for 1 h at 37° C. Reactions were incubated for 20 min at 65° C. to heat-inactivate SapI, cooled down to room temperature and constructs were ligated by addition of 1.1 μl 10×T4 ligase buffer (NEB) and 0.25 μl T4 ligase (NEB) and incubation for 1 h at 25° C. Ligation was stopped by heat-inactivation for 20 min at 65° C. followed by cooling to room temperature. 41 ligation reactions were transformed into chemically-competent E. coli (NEB Stable Competent) and plated on 5% sucrose-containing LB-Ampicillin, yielding pDXinit-CD4Nb-PAC, pDXinit-CD8Nb-PAC and pDXinit-CD16Nb-PAC. For cloning of CD8 hashtag phagemids, 20 ng pDXinit-CD8Nb-PAC were used as template for site-directed mutagenesis (as described earlier in this section) using primers EF156 and EF157 to generate pDXinit-CD8Nb(PH-A)-PAC, primers EF158 and EF159 for pDXinit-CD8Nb(PH-B)-PAC, primers EF164 and EF165 for pDXinit-CD8Nb(PH-C)-PAC and primers EF166 and EF167 for pDXinit-CD8Nb(PH-D)-PAC. For cloning of EGFP Nb-displaying phagemids, the EGFP Nb sequence from pOPINE GFP nanobody (Addgene ID: 49172) was amplified in 50 μl PCR reactions with Q5 (NEB) using 25 ng plasmid template and EF05 and EF06 primers. The EGFP nanobody insert was cloned into pDXinit using FX cloning (described earlier), yielding pDXinit-EGFPNb. EGFP Nb-displaying phagemids containing RD1 in different orientations were cloned by using pDXinit-EGFPNb and performing site-directed mutagenesis (described earlier) with EF73 and EF74 to obtain pDXinit-EGFPNb-PAC or using EF75 and EF76 yielding pDXinit-EGFPNb-RD1(5-3). For introduction of a PCR handle required for PDT library amplification, pDXinit-EGFPNb-PAC was subjected to site-directed mutagenesis (as described earlier in this section) using primers EF78 and EF79, yielding pDXinit-EGFPNb(handle)-PAC. For cloning of mCherry Nb-displaying phagemids, the mCherry Nb sequence from pGex6P1 mCherry nanobody (Addgene ID: 70696) was amplified in 50 μl PCR reactions with Q5 (NEB) using 25 ng plasmid template and EF07 and EF08 primers. The mCherry nanobody insert was cloned into pDXinit using FX cloning (as described earlier in this section), yielding pDXinit-mCherryNb. All constructs are listed in TABLE 4.

TABLE 3

| Name | SEQUENCE (5′-3′) | source |
|---|---|---|
| CD4Nb | ATGGAAGTTCAACTT | https://patentimages. |
| | GTAGAGAGCGGAGGT | storage.googleapis. |
| | GGCTCAGTCCAGCCA | com/09/a8/16/ |
| | GGGGGATCGCTCACA | db148c50e5a90b/ |
| | CTTAGTTGCGGTACT | US20160251440A1.pdf |
| | TCCGGACGAACGTTC | |
| | AATGTTATGGGGTGG | |
| | TTTCGTCAAGCACCT | |
| | GGAAAGGAGCGGGAA | |
| | TTTGTCGCCGCTGTA | |
| | CGGTGGTCATCTACT | |
| | GGAATATATTACACG | |
| | CAATACGCAGATAGC | |
| | GTTAAATCGCGATTT | |
| | ACTATCAGTCGGGAT | |
| | AATGCCAAGAACACT | |
| | GTATATCTGGAAATG | |
| | AACAGCCTGAAACCG | |
| | GAAGATACCGCGGTG | |
| | TATTATTGCGCTGCA | |
| | GATACTTATAATTCA | |
| | AACCCAGCTAGATGG | |
| | GATGGATATGATTTT | |
| | TGGGGCCAGGGCACC | |
| | CAGGTCACCGTGAGC | |
| | AGC | |
| | (SEQ ID NO: 49) | |
| CD16Nb | ATGGAAGTTCAACTT | Genbank EF561291 |
| | GTAGAGAGCGGAGGT | |
| | GAGCTTGTACAAGCA | |
| | GGTGGATCACTTAGA | |
| | CTATCTTGCGCAGCT | |
| | TCCGGGCTCACATTT | |
| | AGTTCGTACAATATG | |
| | GGGTGGTTCCGTAGG | |
| | GCACCAGGTAAGGAG | |
| | CGTGAATTTGTCGCA | |
| | AGTATAACGTGGTCA | |
| | GGACGTGACACTTTT | |
| | TACGCGGATTCCGTA | |
| | AAAGGGCGATTTACG | |
| | ATCAGTCGTGATAAC | |
| | GCTAAGAATACGGTC | |
| | TATCTTCAAATGTCA | |
| | AGTCTAAAACCTGAA | |
| | GATACCGCGGTGTAT | |
| | TATTGCGCAGCTAAT | |
| | CCATGGCCTGTCGCC | |
| | GCACCAAGAAGCGGT | |
| | ACGTATTGGGGCCAG | |
| | GGCACCCAGGTAACA | |
| | GTGAGCAGC | |
| | (SEQ ID NO: 50) | |
| CD8Nb | ATGCAGGTCCAGCTC | https://patentimages. |
| | CAAGAGTCTGGAGGT | storage.googleapis. |
| | GGTTCTGTCCAACCA | com/a0/66/6b/ |
| | GGAGGTTCACTACGT | c5fa3ff38f4c41/ |
| | CTAAGCTGCGCAGCT | WO2017134306A1.pdf |
| | TCCGGTTTCACCTTC | |
| | GACGATTATGCGATG | |
| | TCTTGGGTACGCCAG | |
| | GTTCCTGGAAAGGGA | |
| | TTAGAGTGGGTCTCG | |
| | ACCATCAACTGGAAC | |
| | GGAGGTTCTGCAGAA | |
| | TATGCAGAGCCTGTC | |
| | AAAGGACGTTTCACA | |
| | ATTTCGCGGGACAAC | |
| | GCTAAAAATACTGTA | |
| | TATTTACAGATGAAT | |
| | AGTTTGAAGCTGGAA | |
| | GATACCGCGGTGTAT | |
| | TATTGCGCCAAAGAT | |
| | GCGGACCTGGTATGG | |
| | TACAACCTGTCAACC | |
| | GGACAAGGAACACAA | |
| | GTAACGGTGTCGAGC | |
| | (SEQ ID NO: 51) | |

TABLE 4

| Name | Source |
|---|---|
| pDXinit | Addgene ID: 110101 |
| pDXinit-PAC | Examples 1 and 2 |
| pDXinit-CD4Nb-PAC | Examples 1 and 2 |
| pDXinit-CD16Nb-PAC | Examples 1 and 2 |
| pDXinit-CD8Nb-PAC | Examples 1 and 2 |
| pDXinit-CD8Nb(PH-A)-PAC | Examples 1 and 2 |
| pDXinit-CD8Nb(PH-B)-PAC | Examples 1 and 2 |
| pDXinit-CD8Nb(PH-C)-PAC | Examples 1 and 2 |
| pDXinit-CD8Nb(PH-D)-PAC | Examples 1 and 2 |
| pOPINE GFP nanobody | Addgene ID: 49172 |
| pDXinit-EGFPNb | Examples 1 and 2 |
| pDXinit-EGFPNb-PAC | Examples 1 and 2 |
| pDXinit-EGFPNb-RD1(5-3) | Examples 1 and 2 |
| pDXi nit-EGFPNb (handle)-PAC | Examples 1 and 2 |
| pGex6P1 mCherry nanobody | Addgene ID: 70696 |
| pDXinit-mCherryNb | Examples 1 and 2 |

### Analysis of RD1-Mediated Phagemid Amplification Using RD1-Containing Primers

5 ng of either pDXinit-EGFPNb, pDXinit-EGFPNb-PAC or pDXinit-EGFPNb-RD1(5-3) were subjected to linear PCR (10 μl reaction volume) using primer EF170 and 5 μl 2×KAPA HiFi HotStart ReadyMix (Roche) and cycling conditions 98° C. 2 min; 12 cycles 98° C. 10 sec, 59° C. 30 sec, 72° C. 1 min; final extension 72° C. 5° min. After completion, 0.625 μl of each primer EF147 and EF57, 1.25μl water and 12.5 μl 2×KAPA were added. Nb-specific PCR was performed using 98° C. 3 min; 30 cycles 98° C. 15 sec, 65° C. 20 sec, 72° C. 1 min; final extension 72° C. 5 min. PCR using primers EF57 and EF58 and indicated plasmid templates was used as amplification control.

### Phage Production

Phagemid-containing SS320 (Lucigen) cultures were incubated overnight in 2YT/2%/A/T at 37° C., 240 rpm. Cultures were diluted 1:50 in 2YT/2%/A/T and grown for 2-3 h at 37° C., 240 rpm until OD600=0.4-0.5. 5 ml bacteria were then infected with 200μl M13K07 helper phage (NEB) and incubated for 60 min at 37° C. Bacteria were collected by centrifugation and resuspended in 50 ml 2YT containing 50 μg/ml Ampicillin and 25 μg/ml Kanamycin (2YT/A/K). Phages were produced overnight by incubation at 37° C., 240 rpm. Cultures were centrifuged and phages were precipitated from supernatants by addition of ¼th volume 20% PEG-6000/2.5M NaCl solution and incubation on ice for 75 min. Phages were collected by centrifugation (17 min, 12500 g, 4° C.). Phage pellets were resuspended in 1.2 ml PBS, suspensions were cleared (5 min, 12500 g, 25° C.) and supernatants containing phages were stored.

### Cell Culture

NIH3T3 and HEK293T (ATCC) were maintained in DMEM containing 10% FBS, 2 mM L-glutamine and 100 U/ml penicillin/streptomycin (Thermo Scientific) and cultured at 37° C. and 5% CO2. For sub-culturing, medium was aspirated, cells were washed with PBS and detached with Trypsin-EDTA 0.25% (Thermo Scientific). Detachment reactions were stopped with culture medium and cells were seeded at desired densities. Cell stocks were prepared by resuspending cell aliquots in FBS with 10% DMSO and freezing them slowly at −80° C. Frozen aliquots were then moved to liquid nitrogen for long-term storage. All cell lines were regularly tested for mycoplasma contamination.

### Plasmid Transfection of HEK293T Cells

One day before transfection, 2×10⁶ HEK293T cells were seeded in 10 cm dishes (Corning) in complete culture medium (as described in section ‘Cell culture’). Transfection was performed using GeneJuice reagent (Fisher Scientific). 600 μl Opti-MEM and 12 μl GeneJuice were mixed in 1.5 ml tubes, vortexed shortly and spun down. 4 μg of plasmid DNA (either pCAG (Addgene ID: 11160), pCAC-EGFP (Addgene ID: 89684) or pCAC-EGFP (Addgene ID: 32601)) were added, tubes were vortexed shortly and spun down. Transfection mix was added dropwise to HEK293T cells. Cells were grown for 24 h at 37° C. and 5% CO2 to allow transgene expression. Successful transfection was assessed by fluorescence microscopy on an EVOS M5000.

### Flow Cytometry for Detection of Phage Binding

Harvested cell lines or thawed PBMCs (see PHAGE-ATAC workflow for harvest and thawing protocol) were resuspended in FC buffer (see above) and incubated with respective phage nanobodies for 20 min on a rotator at 4° C. Cells were centrifuged and washed with cold FC buffer twice to remove unbound phages (centrifugation steps all were 350 g, 4 min, 4° C.). For optimization of fixation and lysis conditions, cells were fixed using indicated formaldehyde concentrations (Thermo Scientific) and permeabilized with depicted lysis buffers. Cells were resuspended in FC buffer and anti-M13 antibody (Sino Biological, 11973-MM05T-50) was added at 1:500 dilution. After 10 min on ice, cells were washed twice in FC buffer and anti-mouse Fc Alexa Fluor 647-conjugated secondary antibody (Thermo Scientific, A-21236) was added at 1:500 dilution. Cells were incubated for 10 min on ice, washed twice in FC buffer and resuspended in Sytox Blue (Thermo Scientific) containing FC buffer for live/dead discrimination according to manufacturer's instructions. In indicated cases, cells were stained with anti-CD4-FITC (clone OKT4, BioLegend) at 1:500 dilution, hereby no anti-M13 and anti-mouse Fc antibodies were used. Stained cells were analyzed using a CytoFLEX LX Flow Cytometer (Beckman Coulter) at the Broad Institute Flow Cytometry Facility. Flow cytometry data were analyzed using FlowJo software v.10.6.1.

### PHAGE-ATAC Workflow

For cell line “species mixing” experiment, culture medium was aspirated, cell lines were washed with PBS, harvested using Trypsin-EDTA 0.25% (Thermo Scientific), resuspended in DMEM containing 10% FBS, centrifuged, washed with PBS and resuspended in FC buffer. For PBMC and CD8 T cell experiments, cryopreserved PBMCs or CD8 T cells (AllCells) were thawed, washed in PBS and resuspended in cold Flow cytometry buffer (FC buffer; PBS containing 2% FBS). All centrifugation steps were carried out at 350 g, 4 min, 4° C. unless stated otherwise.

Cells were incubated with phages on a rotating wheel for 20 min at 4° C. After three washes in FC buffer, cells were fixed in PBS containing 1% formaldehyde (Thermo Scientific) for 10 min at room temperature. Fixation was quenched by addition of 2.5M glycine to a final concentration of 0.125M. Cells were washed twice in FC buffer and permeabilized using lysis buffer (10 mM Tris-HCl pH 7.5, 10 mM NaCl, 3 mM MgCl2, 0.1% NP-40, 1% BSA) for 3 min on ice. This buffer was used, as we found that standard 10× Genomics scATAC lysis buffer results in loss of pNb cell staining (FIGS. 11A-11B). After lysis, cells were washed by addition of 1 ml cold wash buffer (lysis buffer without NP-40), inverted and centrifuged (5 min, 500 g, 4° C.). Supernatant was aspirated and the cell pellet was resuspended in 1× Nuclei Dilution Buffer (10× Genomics). Cell aliquots were mixed with Trypan Blue and counting was performed using a Countess II FL Automated Cell Counter. Processing of cells for tagmentation, loading of 10× Genomics chips and droplet encapsulation via the 10× Genomics Chromium controller microfluidics instrument was performed according to Chromium Single Cell ATAC Solution protocol.

For species-mixing, a single 10× channel was ‘super-loaded’ with 20,000 cells. Linear amplification and droplet-based indexing were performed as described in the 10×ATAC protocol on a C1000 Touch Thermal cycler with 96-Deep Well Reaction Module (BioRad). After linear PCR, droplet emulsions were broken, barcoded products were purified using MyONE silane bead cleanup and eluted in 40μl elution buffer I (Chromium Single Cell ATAC Solution protocol). At this point eluates were split for PDT and ATAC library preparation. Whereas 5μl eluate were used for PDT library preparation as described below, the remaining 35μl eluate were used for scATAC library generation (according to Chromium Single Cell ATAC Solution protocol). Splitting samples at this point is not expected to result in a loss of library complexity as PDTs and ATAC fragments already underwent amplification via linear PCR.

The aliquot for PDT library preparation was used for PDT-specific PCR in a 100 μl reaction using 2×KAPA polymerase and primers EF147 and EF91, cycling conditions were: 95° C. 3 min, 20 cycles 95° C. 20 sec, 60° C. 30 sec, 72° 20 sec; final extension 72° C. 5 min. Amplified PDT products were purified by addition of 65μl SPRIselect beads (Beckman Coulter), 160μl supernatants were saved and incubated with 192μl SPRIselect. Beads were washed twice with 800 μl 80% ethanol and the PDT library was eluted in 40μl buffer EB (Qiagen).

Concentration of PDT libraries was determined and 15 ng were used for 100 μl indexing PCR reactions using 50μl Amp-Mix (10× Genomics), 7.5 μl SI-PCR Primer B (10× Genomics) and 2.5 μl i7 sample index-containing primers (10× Genomics), cycling conditions were: 98° C. 45 sec; 6 cycles 98° C. 20 sec, 67° C. 30 sec, 72° 20 sec; final extension 72° C. 1 min. Indexed PDT libraries were purified by addition of 120μl SPRIselect and eluted in 40μl buffer EB. The concentration of final libraries was determined using a Qubit dsDNA HS Assay kit (Invitrogen) and size distribution was examined by running a High Sensitivity DNA chip on a Bioanalyzer 2100 system (Agilent).

PDT and ATAC libraries were pooled and paired-end sequenced (2×34 cycles) using Nextseq High Output Cartridge kits on a Nextseq 550 machine (Illumina). Raw sequencing data were demultiplexed with CellRanger-ATAC mkfastq. ATAC fastqs were used for alignment to the GRCh38 or mm10 reference genomes using CellRanger-ATAC count version 1.0.

### Analysis of RD1-Mediated Phagemid Amplification Using RD1-Containing Primers

5 ng of either pDXinit-EGFPNb, pDXinit-EGFPNb-PAC or pDXinit-EGFPNb-RD1(5-3) were subjected to linear PCR (10 μl reaction volume) using primer EF170 and 5 μl 2×KAPA KAPA HiFi HotStart ReadyMix (Roche) and cycling conditions 98° C. 2 min; 12 cycles 98° C. 10 sec, 59° C. 30 sec, 72° C. 1 min; final extension 72° C. 5° min. After completion, 0.625 μl of each primer EF147 and EF57, 1.25 μl water and 12.5 μl 2×KAPA were added. Nb-specific PCR was performed using 98° C. 3 min; 30 cycles 98° C. 15 sec, 65° C. 20 sec, 72° C. 1 min; final extension 72° C. 5 min. PCR using primers EF57 and EF58 and indicated plasmid templates was used as amplification control.

### PHAGE-ATAC Workflow

For PBMC and CD8 T cell experiments, cryopreserved PBMCs or CD8 T cells (AllCells) were thawed, washed in PBS and resuspended in cold Flow cytometry buffer (FC buffer; PBS containing 2% FBS). For cell line mixing, culture medium was aspirated, cell lines were washed with PBS, harvested using Trypsin-EDTA 0.25% (Thermo Scientific), resuspended in DMEM containing 10% FBS, centrifuged, washed with PBS and resuspended in FC buffer. All centrifugation steps were carried out at 350 g, 4 min, 4° C. unless stated otherwise. Cells were incubated with phages on a rotating wheel for 20 min at 4° C. After three washes in FC buffer, cells were fixed in PBS containing 1% formaldehyde (Thermo Scientific) for 10 min at room temperature. Fixation was quenched by addition of 2.5M glycine to a final concentration of 0.125M. Cells were washed twice in FC buffer and permeabilized using lysis buffer (10 mM Tris-HCl pH 7.5, 10 mM NaCl, 3 mM MgCl₂, 0.1% NP-40, 1% BSA) for 3 min on ice. This buffer was used, as we found that standard 10×scATAC lysis buffer results in loss of pNb cell staining (FIGS. 11A-11B). After lysis, cells were washed by addition of 1 ml cold wash buffer (lysis buffer without NP-40), inverted and centrifuged (5 min, 500 g, 4° C.). Supernatant was aspirated and the cell pellet was resuspended in 1× Nuclei Dilution Buffer (10× Genomics). Cell aliquots were mixed with Trypan Blue and counting was performed using a Countess II FL Automated Cell Counter. Processing of cells for tagmentation, loading of 10× chips and droplet encapsulation via the 10× Chromium controller microfluidics instrument was performed according to Chromium Single Cell ATAC Solution protocol. For species-mixing, a single 10× channel was ‘super-loaded’ with 20,000 cells. Linear amplification and droplet-based indexing were performed as described in the 10×ATAC protocol on a C1000 Touch Thermal cycler with 96-Deep Well Reaction Module (BioRad). After linear PCR, droplet emulsions were broken, barcoded products were purified using MyONE silane bead cleanup and eluted in 40 μl elution buffer I (Chromium Single Cell ATAC Solution protocol). At this point eluates were split for PDT and ATAC library preparation. Whereas 5 μl eluate were used for PDT library preparation as described below, the remaining 35 μl eluate were used for ATAC library generation (according to Chromium Single Cell ATAC Solution protocol). Splitting samples at this point is not expected to result in a loss of library complexity as PDTs and ATAC fragments already underwent amplification via linear PCR. The aliquot for PDT library preparation was used for PDT-specific PCR in a 100 μl reaction using 2×KAPA polymerase and primers EF147 and EF91, cycling conditions were: 95° C. 3 min, 20 cycles 95° C. 20 sec, 60° C. 30 sec, 72° 20 sec; final extension 72° C. 5 min. Amplified PDT products were purified by addition of 65 μl SPRIselect beads (Beckman Coulter), 160 μl supernatants were saved and incubated with 192 μl SPRIselect. Beads were washed twice with 800 μl 80% ethanol and the PDT library was eluted in 40 μl buffer EB (Qiagen).

Concentration of PDT libraries was determined and 15 ng were used for 100 μl indexing PCR reactions using 50 μl Amp-Mix (10× Genomics), 7.5 μl SI-PCR Primer B (10× Genomics) and 2.5 μl i7 sample index-containing primers (10× Genomics), cycling conditions were: 98° C. 45 sec; 6 cycles 98° C. 20 sec, 67° C. 30 sec, 72° 20 sec; final extension 72° C. 1 min. Indexed PDT libraries were purified by addition of 120 μl SPRIselect and eluted in 40 μl buffer EB. The concentration of final libraries was determined using a Qubit dsDNA HS Assay kit (Invitrogen) and size distribution was examined by running a High Sensitivity DNA chip on a Bioanalyzer 2100 system (Agilent). PDT and ATAC libraries were pooled and paired-end sequenced (2×34 cycles) using Nextseq High Output Cartridge kits on a Nextseq 550 machine (Illumina). Raw sequencing data were demultiplexed with CellRanger-ATAC mkfastq. ATAC fastqs were used for alignment to the GRCh38 or mm10 reference genomes using CellRanger-ATAC count version 1.0.

### Computational Workflow for Generation of PDT Count Matrices

PDT fastqs were obtained by running CellRanger-ATAC mkfastq on raw sequencing data and custom UNIX code was used to derive PDT-cell barcode count tables. For each lane, using ‘grep -B1’ function, PDT_R3 fastqs were searched for each CDR3 barcode sequence (Table 5) and corresponding sequencing cluster information was derived. Cluster information was used to derive corresponding cell barcodes from PDT_R2 fastqs by using ‘fgrep -A1 -f’. Files containing identified cell barcodes from all four lanes were concatenated, the reverse complement of cell barcode sequences was generated using ‘tr ACGTacgt TGCAtgca’ (SEQ ID NO: 52) and barcodes were filtered via ‘fgrep -f’ using the cell barcodes called by CellRanger-ATAC count. Unique cell barcode occurrences were counted.

TABLE 5

| | Sequence (5′-3′), | |
|---|---|---|
| | read for barcode | |
| | readout is | SEQ |
| | indicated in | ID |
| Name | brackets | NO: |
| CD8Nb PH-A | GATACCGCGGTGTAT | 53 |
| | TATTGCGCAAAGGAC | |
| | GCGG (R3) | |
| CD8Nb PH-B | GATACCGCGGTGTAT | 54 |
| | TATTGCGCTAAAGAC | |
| | GCGG (R3) | |
| CD8Nb PH-C | CAGCTCTTCCTGCG | 55 |
| | CTGCTTACCGTAAC | |
| | TTGTGT (R1) | |
| CD8Nb PH-D | CAGCTCTTCCTGCGC | 56 |
| | TGCTTACAGTGACCT | |
| | GTGT (R1) | |
| CD8Nb | GATACCGCGGTGTAT | 57 |
| | TATTGCGCCAAAGAT | |
| | GCGG (R3) | |
| CD4Nb | GATACCGCGGTGTAT | 58 |
| | TATTGCGCTGCAGAT | |
| | ACTT (R3) | |
| CD16Nb | GATACCGCGGTGTAT | 59 |
| | TATTGCGCAGCTAAT | |
| | CCAT (R3) | |
| EGFPNb | GATACCGCGGTGTAT | 60 |
| | TATTGCAATGTCAAC | |
| | GTGG (R3) | |

### Analysis of Species Mixing PHAGE ATAC Experiment

PHAGE-ATAC sequencing data from the species-mixing experiment was demultiplexed using CellRanger-ATAC mkfastq and generated ATAC fastqs were processed with CellRanger-ATAC count to filter reads, trim adapters, align reads to both GRCh38 and mm10 reference genomes, count barcodes, identify transposase cut sites, detect accessible chromatin peaks and to identify cutoffs for cell barcode calling. The “force-cells” parameter was not set. Barcodes were classified as human or mouse if >90% of barcode-associated fragments aligned to GRCh38 or mm10, respectively. Cutoffs for cell barcode calling were >3,000 ATAC fragments overlapping peaks for human and >10,000 for mouse barcodes (based on empirical density). Doublet barcodes were defined as containing more than 10% ATAC fragments aligning to both GRCh38 and mm10 reference genomes. The EGFP PDT count table was generated as described above by searching PDT fastqs for the corresponding phage barcode (Table 5) and deriving PDT-associated cell barcodes via filtering using the entire list of called cell barcodes (human and mouse).

After flow cytometry measurement of HEK293T-EGFP-GPI (EGFP+) and HEK293T cells (EGFP−), FCS files were exported using CytExpert Software (Beckman Coulter). Values for forward scatter (FSC area) and EGFP fluorescence (FITC area) were derived from FCS files. Human EGFP+ and EGFP-cells were defined based on the distribution of EGFP PDT counts (for PHAGE-ATAC) or EGFP fluorescence represented by FITC-area values (for flow cytometry) by setting a gate at the minimum value in-between both populations

### Analysis of PBMC PHAGE-ATAC Experiment

Sequencing data from two libraries of PBMCs were processed using CellRanger-ATAC count to the GRChg38 reference genome using all default parameters, yielding 7,792 high-quality PBMCs (no filtering was applied beyond the CellRanger-ATAC knee call). We downloaded processed CITE-seq PBMC data (Stoeckius et al., 2017) from the Gene Expression Omnibus (GSE100866). After removing spiked-in mouse cells, this published dataset was jointly analyzed with the 7,972 PBMCs profiled by PHAGE-ATAC. Applicant performed data integration using canonical correlation analysis (Butler et al., 2018), using the 2,000 most variable RNA genes as is the default in Seurat. Next, Applicant performed RNA imputation for the ATAC-seq data using Seurat v3 with the default settings (Stuart et al., 2019). Reduced dimensions and cell clusters were inferred using this merged object via the first 20 canonical correlation components with the default Louvain clustering in Seurat v3. Centered log ratio (CLR) normalized PDTs were visualized in the reduced dimension space and a per-tag, per-cluster mean was further computed to further access staining efficiency between the modalities (FIG. 1N)

Cell annotations were derived based on well-established marker genes for PBMCs (Supp. FIG. 10A), and the granulocyte population was corroborated by high overall fragments but low proportion of fragments overlapping chromatin accessibility peaks. For protein-based clustering and analyses, we identified T-cell clusters from the integrated embedding (using the chromatin/RNA data) and then further stratified into subpopulations based on the abundance of the CD4 and CD8 CLR PDT (FIG. 12B). Differential gene activity scores between these populations were then computed using the default functionality in Seurat/Signac (Wilcoxon rank-sum test).

### Analysis of Cell Hashing PHAGE-ATAC Experiment

One channel of sequencing data from the hashed, combined CD8-enriched T cells was processed using CellRanger-ATAC count via the GRCh38 reference genome using all default parameters, yielding 8,366 high-quality PBMCs (no filtering was applied beyond the CellRanger-ATAC knee call). As Applicant suspected the presence of contaminating B-cells, Applicant first characterized cell states using latent semantic indexing (LSI)-based clustering and dimensionality reduction using Signac and Seurat (Stuart et al., 2019). Specifically, all detected peaks were used as input into LSI. The first 20 LSI components (except for the first component, which was found to be correlated with the per-cell sequencing depth) were used to define cell clusters using the default Louvain clustering algorithm in Seurat. Per-cluster chromatin accessibility tracks were computed using a per million fragments abundance for each cluster, as previously implemented (Lareau et al., 2020). These chromatin accessibility tracks were used to annotate cell clusters based on promoter accessibility of known marker genes.

To assign hash identities to cell barcodes, we utilized the HTODemux function from Seurat (Stoeckius et al., 2018) with the positive.quantile parameter set at 0.98. This yielded 703 doublets, 1,225 negatives, and 6,438 singlets based on the abundance and distribution of CD8 hashtag PDTs.

To verify PHAGE-ATAC hashtag-based assignments, Applicant performed mitochondrial DNA genotyping using mgatk (Lareau et al., 2020) and nuclear genotyping and donor assignment using souporcell (Heaton et al., 2020) with “--min_alt 8 --min_ref 8 --no_umi True -k 4 --skip_remap True --ignore True” options, which resulted in 92.9% accuracy (99.3% singlet accuracy, 74% overlap in called doublets), confirming the concordance of our hashing design.

### Cloning of PANL, a Synthetic High-Complexity Phage Nanobody Library

To generate randomized library inserts, three separate primer mixes (for long CDR3, medium CDR3 and short CDR3 inserts) were used for PCR-mediated assembly. For short CDR3-inserts, the primer mix contained 0.5 μl each of polyacrylamide gel electrophoresis-purified EF42, EF43, EF64, EF44, EF65, EF45, EF46, EF47, EF66 and EF48 (each 100 μM) (EllaBiotech). For medium CDR3-inserts, EF67 was used instead of EF66. For long CDR3-inserts, EF68 was used instead of EF66. Primer mixes were diluted 1:25 and 1 μl of each mix was used for overlap-extension PCR using Phusion (NEB). Four 50μl reactions for each mix were performed using cycling conditions 98° C. 1 min; 20 cycles 98° C. 15 sec, 60° C. 30 sec, 72° 30 sec; final extension 72° C. 5 min. PCR reactions of the same mix were pooled and purified by addition of 280 μl AMPure XP beads (Beckman Coulter). Beads were washed twice with 800 μl 80% ethanol and assembled inserts were eluted in 100 μl water. Concentrations of each insert (long, medium, short) were determined and pooled in a 1:2:1 molar ratio. Five identical 50μl PCR reactions with pooled inserts and primers EF40 and EF41 were performed using Phusion (NEB), cycling conditions were 98° C. 1 min; 30 cycles 98° C. 15 sec, 62° C. 30 sec, 72° 30 sec; final extension 72° C. 5 min. Amplified library insert was pooled and purified by adding 350μl AMPure XP beads (Beckman Coulter). Beads were washed twice with 1 ml 80% ethanol and library insert was eluted in 60μl water. Five identical 60μl restriction digest reactions for digest of 7.5 μg library vector pDXinit-PAC with 2.5 μl SapI were performed. Library insert (4.8 μg) was digested in a 30μl reaction using 2.5 μl SapI. Digests were incubated for 4 h at 37° C. and loaded on 1% agarose gels. Bands corresponding to digested library vector and insert were cut and products were extracted using GeneJet Gel Extraction Kit (Thermo Scientific) and eluted in 40μl water. Five identical 100 μl ligation reactions were performed, each containing 1.25 μg digested pDXinit-PAC, 450 ng digested insert and 0.5 μl T4 ligase (NEB). Ligations were incubated for 16 h at 16° C., heat-inactivated for 20 min at 65° C. and cooled to room temperature. 100 μl AMPure XP beads were added to each ligation reaction, beads were washed twice using 300 μl 80% ethanol and ligation products were eluted in 15 μl water and pooled. Five electroporations in 2 mm cuvettes (BioRad) were performed, each using 90μl electro-competent SS320 E. coli (Lucigen) and 12μl ligation product. Pulsing was performed on a GenePulserXcell instrument (BioRad) with parameters 2.5 kV, 200 Ohm, 25 μF. After electroporation, bacterial suspensions were added to 120 ml pre-warmed SOC and incubated for 30 min, 37° C., 225 rpm. An aliquot of library-carrying bacteria was saved at this point and used to prepare a dilution series. Each dilution was plated on LB-Ampicillin plates. After overnight incubation at 37° C., colonies were counted, transformation efficiency was determined and library complexity was estimated. The remaining 120 ml of library-containing culture were added to 1.125 L 2YT/2%/A/T and incubated overnight at 37° C., 240 rpm. The library-containing culture was harvested, glycerol stocks were prepared and library aliquots were stored.

### Analysis of Picked PANL Clones Using PCR and Sanger Sequencing

Library-containing bacteria were plated on LB-Ampicillin, incubated overnight, colonies were picked and inoculated in 8 ml LB-Ampicillin. Cultures were incubated for at least 8 h at 37° C., 240 rpm. Bacteria were harvested and plasmids isolated using GeneJet Plasmid Miniprep kit (Thermo Scientific). PCR was performed to evaluate clone inserts. 10 μl PCR reactions were set up that contained 10 ng of isolated plasmid, 0.5 μl each of primers EF52 and EF53, and 4.5 μl 2× OneTaq Quick Load Master Mix (NEB). Cycling conditions were 94° C. 4 min; 28 cycles 94° C. 15 sec, 62° C. 15 sec, 68° C. 30 sec; final extension 68 C 5 min. PCR reactions were analyzed on 2% agarose gels. Selected clones were analyzed by Sanger Sequencing using primer EF17. Observed amino acid frequencies at hypervariable positions were assessed by analyzing Sanger sequences of 25 picked clones.

### Phage Nanobody Library Production

A PANL aliquot corresponding to 3×10¹⁰ bacterial cells (around 5× coverage of the library) was transferred to 200 ml 2YT/2%/A/T and cultures were grown until OD600=0.5 was reached (about 2 h). Cultures were infected with 8 ml M13K07 helper (NEB) for 60 min at 37° C. Cultures were harvested, supernatants discarded and bacterial pellets were resuspended in 1 L 2YT/A/K. Cultures were incubated overnight at 37° C., 250 rpm for production of the input library of phage nanobody particles. Bacterial cultures were harvested, supernatants collected and phages were precipitated using PEGNaCl as described earlier. Final phage pellets were resuspended in a total of 20 ml PBS and stored. Phage titers were determined by infecting a log-phase culture of SS320 with a dilution series of the produced phage library and plating bacteria on LB-Ampicillin. Colonies were counted and titers were calculated. Produced phage libraries were characterized by titers >4×10¹¹ pfu/ml.

### Phage Display Selection

HEK293T cells were transfected either with pCAG or pCAG-EGFP-GPI as described above. Cells were harvested, 107 pCAG-transfected cells were resuspended in 1 ml PBS containing 2% BSA (PBS-BSA), and 8 ml PANL library (1.6×1012 pfu) in PBS-BSA were added for counter-selection. Samples were incubated for 1 h on a rotating wheel at 4° C. and then centrifuged at 350 g, 5 min, 4° C. Supernatants containing phages were added to 107pCAG-EGFP-GPI expressing cells for positive selection. After 1 h on a rotating wheel at 4° C., samples were centrifuged (350 g, 5 min, 4° C.) and washed 6 times with PBS-BSA to remove unbound phages. Cells were washed once in PBS, centrifuged and cell pellets were resuspended in 500μl Trypsin solution (1 mg/ml Trypsin (Sigma Aldrich) in PBS) to elute bound phages. Cells were incubated for 30 min on a rotating wheel at room temperature and digests were stopped by addition of AEBSF protease inhibitor (Sigma Aldrich) to a final concentration of 0.5 mg/ml. Samples were centrifuged (400 g, 4 min at room temperature) and the supernatant containing eluted phages was used to infect 10 ml of log-phase SS320 (OD600=0.4). After infection for 40 min at 37° C., cultures were added to 90 ml 2YT/2%/A/T and incubated overnight at 37° C., 250 rpm. Cultures containing output libraries were aliquoted and glycerol stocks were prepared. Output library phage particles were prepared as described earlier for PANL and used in subsequent selection rounds using the same protocol described here.

### REFERENCES RELATED TO EXAMPLES 1 AND 2

Butler, A., Hoffman, P., Smibert, P., Papalexi, E., and Satija, R. (2018). Integrating single-cell transcriptomic data across different conditions, technologies, and species. Nat Biotechnol 36, 411-420.
Gebauer, M., and Skerra, A. (2009). Engineered protein scaffolds as next-generation antibody therapeutics. Curr Opin Chem Biol 13, 245-255.
Geertsma, E. R., and Dutzler, R. (2011). A versatile and efficient high-throughput cloning tool for structural biology. Biochemistry 50, 3272-3278.
Gehring, J Hwee Park, J., Chen, S., Thomson, M., and Pachter, L. (2020). Highly multiplexed single-cell RNA-seq by DNA oligonucleotide tagging of cellular proteins. Nat Biotechnol 38, 35-38.
Heaton, H., Talman, A. M., Knights, A., Imaz, M., Gaffney, D. J., Durbin, R., Hemberg, M., and Lawniczak, M. K. N. (2020). Souporcell: robust clustering of single-cell RNA-seq data by genotype without reference genotypes. Nat Methods 17, 615-620.
Hoogenboom, H. R. (2005). Selecting and screening recombinant antibody libraries. Nat Biotechnol 23, 1105-1116.
Ingram, J. R., Schmidt, F. I., and Ploegh, H. L. (2018). Exploiting Nanobodies' Singular Traits. Annu Rev Immunol 36, 695-715.
Katzenelenbogen, Y., Sheban, F., Yalin, A., Yofe, I., Svetlichnyy, D., Jaitin, D. A., Bornstein, C., Moshe, A., Keren-Shaul, H., Cohen, M., et al. (2020). Coupled scRNA-Seq and Intracellular Protein Activity Reveal an Immunosuppressive Role of TREM2 in Cancer. Cell 182, 872-885 e819.
Klein, A. M., Mazutis, L., Akartuna, I., Tallapragada, N., Veres, A., Li, V., Peshkin, L., Weitz, D. A., and Kirschner, M. W. (2015). Droplet barcoding for single-cell transcriptomics applied to embryonic stem cells. Cell 161, 1187-1201.
Kubala, M. H., Kovtun, O., Alexandrov, K., and Collins, B. M. (2010). Structural and thermodynamic analysis of the GFP:GFP-nanobody complex. Protein Sci 19, 2389-2401.
Lareau, C. A., Duarte, F. M., Chew, J. G., Kartha, V. K., Burkett, Z. D., Kohlway, A. S., Pokholok, D., Aryee, M. J., Steemers, F. J., Lebofsky, R., et al. (2019). Droplet-based combinatorial indexing for massive-scale single-cell chromatin accessibility. Nat Biotechnol 37, 916-924.
Lareau, C. A., Ludwig, L. S., Muus, C., Gohil, S. H., Zhao, T., Chiang, Z., Pelka, K., Verboon, J. M., Luo, W., Christian, E., et al. (2020). Massively parallel single-cell mitochondrial DNA genotyping and chromatin profiling. Nat Biotechnol.
Ludwig, L. S., Lareau, C. A., Ulirsch, J. C., Christian, E., Muus, C., Li, L. H., Pelka, K., Ge, W., Oren, Y., Brack, A., et al. (2019). Lineage Tracing in Humans Enabled by Mitochondrial Mutations and Single-Cell Genomics. Cell 176, 1325-1339 e1322.
Ma, A., McDermaid, A., Xu, J., Chang, Y., and Ma, Q. (2020). Integrative Methods and Practical Challenges for Single-Cell Multi-omics. Trends Biotechnol 38, 1007-1022. Google Scholar
Macosko, E. Z., Basu, A., Satija, R., Nemesh, J., Shekhar, K., Goldman, M., Tirosh, I., Bialas, A. R., Kamitaki, N., Martersteck, E. M., et al. (2015). Highly Parallel Genome-wide Expression Profiling of Individual Cells Using Nanoliter Droplets. Cell 161, 1202-1214. CrossRefPubMedGoogle Scholar
McGinnis, C. S., Patterson, D. M., Winkler, J., Conrad, D. N., Hein, M. Y., Srivastava, V., Hu, J. L., Murrow, L. M., Weissman, J. S., Werb, Z., et al. (2019). MULTI-seq: sample multiplexing for single-cell RNA sequencing using lipid-tagged indices. Nat Methods 16, 619-626.
McMahon, C., Baier, A. S., Pascolutti, R., Wegrecki, M., Zheng, S., Ong, J. X., Erlandson, S. C., Hilger, D., Rasmussen, S. G. F., Ring, A. M., et al. (2018). Yeast surface display platform for rapid discovery of conformationally selective nanobodies. Nat Struct Mol Biol 25, 289-296.
Miersch, S., and Sidhu, S. S. (2012). Synthetic antibodies: concepts, potential and practical considerations. Methods 57, 486-498.
Mimitou, E. P., Cheng, A., Montalbano, A., Hao, S., Stoeckius, M., Legut, M., Roush, T., Herrera, A., Papalexi, E., Ouyang, Z., et al. (2019). Multiplexed detection of proteins, transcriptomes, clonotypes and CRISPR perturbations in single cells. Nat Methods 16, 409-412.
Paul, F., Arkin, Y., Giladi, A., Jaitin, D. A., Kenigsberg, E., Keren-Shaul, H., Winter, D., Lara-Astiaso, D., Gury, M., Weiner, A., et al. (2015). Transcriptional Heterogeneity and Lineage Commitment in Myeloid Progenitors. Cell 163, 1663-1677.
Peterson, V. M., Zhang, K. X., Kumar, N., Wong, J., Li, L., Wilson, D. C., Moore, R., McClanahan, T. K., Sadekova, S., and Klappenbach, J. A. (2017). Multiplexed quantification of proteins and transcripts in single cells. Nat Biotechnol 35, 936-939.
Pollock, S. B., Hu, A., Mou, Y., Martinko, A. J., Julien, O., Hornsby, M., Ploder, L., Adams, J. J., Geng, H., Muschen, M., et al. (2018). Highly multiplexed and quantitative cell-surface protein profiling using genetically barcoded antibodies. Proc Natl Acad Sci USA 115, 2836-2841.
Roobrouck, A., Stortelers, C., Vanlandschoot, P., Staelens, S., Conde, M., Soares, H., and Schols, D. (2016). Bispecific Nanobodies. US 2016/0251440 A1.
Rothbauer, U., Zolghadr, K., Tillib, S., Nowak, D., Schermelleh, L., Gahl, A., Backmann, N., Conrath, K., Muyldermans, S., Cardoso, M. C., et al. (2006). Targeting and tracing antigens in live cells with fluorescent nanobodies. Nat Methods 3, 887-889.
Satpathy, A. T., Granja, J. M., Yost, K. E., Qi, Y., Meschi, F., McDermott, G. P., Olsen, B. N., Mumbach, M. R., Pierce, S. E., Corces, M. R., et al. (2019). Massively parallel single-cell chromatin landscapes of human immune cell development and intratumoral T cell exhaustion. Nat Biotechnol 37, 925-936.
Smith, G. P. (1985). Filamentous fusion phage: novel expression vectors that display cloned antigens on the virion surface. Science 228, 1315-1317.
Stoeckius, M., Hafemeister, C., Stephenson, W., Houck-Loomis, B., Chattopadhyay, P. K., Swerdlow, H., Satija, R., and Smibert, P. (2017). Simultaneous epitope and transcriptome measurement in single cells. Nat Methods 14, 865-868.
Stoeckius, M., Zheng, S., Houck-Loomis, B., Hao, S., Yeung, B. Z., Mauck, W. M., 3rd, Smibert, P., and Satija, R. (2018). Cell Hashing with barcoded antibodies enables multiplexing and doublet detection for single cell genomics.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An engineered display construct comprising:
optionally, a genetically encoded display molecule or a genetically encoded display molecule linker;
a genetically encoded affinity molecule; and
a genetically encoded sequencing molecule, wherein the genetically encoded sequencing molecule is fused to or operatively coupled to the genetically encoded affinity molecule and the genetically encoded display molecule.

2. The engineered display construct of claim 1, wherein the sequencing molecule is a barcode polynucleotide, an index polynucleotide, a primer-binding site, an adapter polynucleotide, or a combination thereof.

3. The engineered display construct of claim 1, wherein the engineered display construct is a viral vector, a non-viral vector, a naked polynucleotide, an expression vector, optionally a prokaryotic expression vector or a eukaryotic cell expression vector, a phagemid, or a system thereof.

4. (canceled)

5. (canceled)

6. The engineered display construct of claim 1, wherein the genetically encoded display molecule is a genetically encoded capsid polypeptide, a genetically encoded prokaryotic cell surface polypeptide, a genetically encoded eukaryotic cell surface polypeptide, a genetically encoded P2A endonuclease polypeptide, or a genetically encoded RepA polypeptide.

7. An engineered display system comprising: the engineered display construct of claim 1.

8. The engineered display system of claim 7, wherein the display system is an engineered viral display system, an engineered prokaryotic cell display system, an engineered eukaryotic cell display system, an engineered mRNA display system, an engineered ribosome display system, or an engineered DNA display system.

9. The engineered display system of claim 7, wherein the engineered display system is an engineered bacteriophage; an engineered non-bacteria virus; an engineered bacterial cell; an engineered yeast cell; an engineered mammalian cell; an engineered insect cell; an engineered DNA display system; an engineered ribosome display system; an engineered covalent display system; or an engineered CIS display system.

10. The engineered display system of claim 7, further comprising:
a display molecule, wherein the display molecule is a optionally a capsid polypeptide and wherein the optional capsid polypeptide is a major capsid polypeptide or a minor capsid polypeptide;
an affinity molecule; and
a sequencing polypeptide,
wherein the sequencing polypeptide is fused to or operatively coupled to the display molecule, the affinity polypeptide, or both.

11. The engineered display system of claim 7, wherein the display molecule comprises a capsid polypeptide, a yeast cell surface polypeptide, a bacteria cell surface polypeptide, a mammalian cell surface polypeptide, an insect cell surface polypeptide, a puromycin, a ribosome or component thereof, a P2A endonuclease polypeptide, or a RepA polypeptide.

12. The engineered display system of claim 7, wherein the affinity molecule comprises a peptide, polypeptide, polynucleotide, a small molecule, or any combination thereof.

13. The engineered display system of claim 7, wherein the affinity molecule is an antibody or fragment thereof, and optionally comprises or consists of a human or humanized antibody VH domain.

14. (canceled)

15. (canceled)

16. (canceled)

17. (canceled)

18. A display construct library comprising:
a plurality of engineered displayed constructs according to claim 1, wherein the plurality of engineered display constructs are engineered phagemids.

19. (canceled)

20. The display construct library of claim 18, wherein each of the engineered display constructs or two or more of the engineered display constructs comprise a unique genetically encoded affinity molecule, a unique genetically encoded display molecule, a unique genetically encoded sequencing molecule, or any combination thereof.

21. (canceled)

22. A plurality of engineered display constructs comprising an engineered display construct library as in claim 18.

23. A method of multi-omic single cell or single nuclei analysis, comprising:
specifically binding one or more individual cells, individual nuclei, or both with an engineered display system or plurality thereof of as in any one of the preceding claims;
allowing each affinity molecule to specifically bind a target molecule present inside of and/or on the surface of the one or more individual cells and/or individual nuclei;
fixing the specifically bound engineered display system(s) to the one or more individual cells and/or individual nuclei;
accessing cellular polynucleotides within one or more individual specifically bound cells and/or individual specifically bound nuclei;
accessing the engineered display construct(s) in the specifically bound engineered display construct(s); and
characterizing one or more features of the one or more individual specifically bound cells and/or individual specifically bound nucleic based, at least in part, on sequencing, in whole or in part,
(i) the accessed genetically encoded affinity molecule, genetically encoded sequencing molecule, or both present in the specifically bound engineered display construct and
(ii) the one or more accessed cellular and/or nuclear polynucleotides, and optionally wherein sequencing comprises a single cell, single nucleus sequencing technique, or both.

24. The method of claim 23, further comprising generating, within one or more individual specifically bound cells and/or nuclei, cDNA copies of cellular RNA molecules.

25. The method of claim 23, wherein characterizing one or more features is based, at least in part, on sequencing the cDNA copies of cellular RNA molecules.

26. The method of claim 23, wherein sequencing comprises sequencing a portion of the accessed genetically encoded affinity molecule, genetically encoded sequencing molecule, or both present in the specifically bound engineered display construct and a portion of each of the one or more accessed cellular, one or more nuclear polynucleotides, or both.

27. The method of claim 23, wherein the step of accessing polynucleotides present inside the individual cell and/or individual nuclei comprises permeabilizing the cell, permeabilizing the nucleus, lysing the cells, lysing the nucleus or a combination thereof.

28. The method of claim 23, further comprising tagmenting, within individual cells and/or individual nuclei, genomic DNA to produced tagmented genomic DNA fragments.

29. The method of claim 23, wherein sequencing comprises sequencing the one or more tagmented genomic DNA fragments or a portion thereof.

30. The method of claim 23, further comprising incorporating a cell or nuclei barcode into the one or more cellular polynucleotides, cDNA copies, tagmented genomic DNA fragments, the genetically encoded affinity molecule, the genetically encoded sequencing molecule, or a combination thereof, such that the one or more cellular polynucleotides, cDNA copies, tagmented genomic DNA fragments, genetically encoded affinity molecule, the genetically encoded sequencing molecule, or a combination thereof from the same cell receive the same unique cell, from the same nuclei receive the same nuclei barcode sequence, or both.

31. The method of claim 23, further comprising incorporating into the one or more cellular polynucleotides, cDNA copies, tagmented genomic DNA fragments, the genetically encoded affinity molecule, the genetically encoded sequencing molecule, or a combination thereof,
a. one or more barcodes;
b. one or more PCR handles;
c. one or more unique molecular identifiers (UMIs);
d. one or more affinity tags;
e. one or more sequencing adapters;
f. one or more linkers;
g. a poly(T) sequence;
h. a poly(A) sequence;
i. one or more primer sites; or
j. any combination thereof.

32. The method of claim 23, further comprising amplifying the one or more cellular polynucleotides, nuclear polynucleotides, cDNA copies, tagmented genomic DNA fragments, the genetically encoded affinity molecule, the genetically encoded sequencing molecule, or a combination thereof.

33. The method of claim 23, further comprising mixing the one or more cellular polynucleotides, cDNA copies, tagmented genomic DNA fragments, the genetically encoded affinity molecule, the genetically encoded sequencing molecule, or a combination thereof with an oligonucleotide-adorned bead, wherein each oligonucleotide on the oligonucleotide-adorned bead comprises:
a. one or more linkers;
b. one or more barcodes;
c. one or more unique molecular identifiers (UMIs);
d. one or more affinity tags;
e. one or more sequencing adapters
f. one or more reaction handles or substrates;
g. one or more primer sites;
h. a poly(T) sequence;
i. a poly(A) sequence;
j. one or more PCR handles; or
k. any combination thereof,
wherein mixing optionally occurs in or on a substrate or a container.

34. The method of claim 23, further comprising isolating a cell and/or nucleus that is specifically bound to and fixed to one or more engineered bacteriophages in or on a substrate, in an individual discrete volume, or container, wherein the container is optionally a well, microwell, capillary, or microcapillary and wherein the individual discrete volume is a liquid, solid, a semi-solid, a gel, a droplet, or a slide.

35. (canceled)

36. (canceled)

37. (canceled)

38. (canceled)

39. The method of claim 33, wherein one or more oligonucleotide-adorned beads are present on a surface of the substrate or container and are arranged in an ordered array, wherein each oligonucleotide-adorned bead has a unique barcode corresponding to the x,y coordinate of the oligonucleotide-adorned bead in the array.

40. The method of claim 39, further comprising depositing a tissue section comprising the one or more individual cells on the ordered array, optionally wherein one or more individual cells are present in a tissue sample and specific binding and fixing occurs in situ.

41. (canceled)

42. The method of 23, wherein sequencing the genetically encoded affinity molecule, the genetically encoded sequencing molecule, or both and sequencing the one or more cellular polynucleotides, one or more nuclear polynucleotides, or both occurs in situ.

43. The method of claim 23, further comprising converting unmethylated cytosines to uracil in the genomic DNA via bisulfite conversion prior to sequencing the genomic DNA or portion thereof.

44. The method of claim 23, wherein the one or more features comprise a cellular RNA expression profile; a surface protein expression profile; an epigenetic feature of a genomic DNA region in the cell; or any combination thereof, optionally wherein the epigenetic feature comprises a profile of chromatin accessibility along the genomic DNA region; a DNA binding protein occupancy for a binding site in the genomic DNA region; a nucleosome-free DNA in the genomic DNA region; a positioning of the nucleosomes along the genomic DNA region; methylation status; chromatin states; or any combination thereof.

45. (canceled)

46. (canceled)

47. The method of claim 23, further comprising diagnosing, monitoring, or prognosing a condition or disease in a subject, wherein diagnosing, monitoring, or prognosing comprises:
characterizing a feature of one or more individual cells in the subject at one or more time points using the method of claim 23; and
providing a diagnosis, prognosis, or condition or disease status based on the one or more characterized features.

48. A method of generating a specific pool of engineered display constructs or engineered display systems having a desired target affinity, comprising:
a. generating an input display construct or engineered display system library, wherein each display construct or display system present in the input library is as in any one of the preceding claims;
b. removing from the input library via negative selection at least some of the engineered display constructs or engineered display systems in the input library that do not specifically bind or otherwise associate with a desired target;
c. positively selecting engineered display constructs or engineered display systems form the pool formed after step (b) that specifically bind or otherwise associate with the desired target; and
d. amplifying the positively selected engineered display constructs or engineered display systems; and
e. optionally sequencing one or more regions of the positively selected engineered display constructs.

49. The method of claim 48, further comprising repeating steps (b) through (c) or through (d) one or more times, wherein the input for step (b) is the output from step (c) or step (d).

50. (canceled)

51. A kit for performing multi-omic single cell analysis, comprising:
an engineered display construct, an engineered display construct library, and/or an engineered display system or plurality thereof.

52. The kit of claim 51, wherein the engineered display construct is as in claim 1.

53. The kit of claim 51, wherein the engineered display construct library is as in claim 18.

54. The kit of claim 51, wherein the engineered display system is as in claim 7.

55. The kit of claim 51, wherein
a. the affinity molecule of each engineered display system is capable of specifically binding a predetermined target present on the surface of, inside of a cell, nucleus, or any combination thereof;
b. the genetically encoded affinity molecule is capable of generating an affinity molecule polypeptide capable of specifically binding a predetermined target present on the surface of, inside of a cell, nucleus, or any combination thereof;
c. the predetermined target is a microorganism protein, a cancer-associated protein, an immune checkpoint inhibitor, a cell-type marker, a cell-state marker, a non-cancer disease or condition biomarker, or any combination thereof; or
d. any combination thereof.

56. (canceled)

57. (canceled)
